(19) United States
(12) Patent Application Publication
(10) Pub. No.: US 2024/0325556 A1
(43) Pub. Date: Oct. 3, 2024

(54) Methods of Using Antibody-Drug-Conjugates

(71) Applicant: R.P. Scherer Technologies, LLC, Carson City, NV (US)

(72) Inventor: Penelope M. Drake, Castro Valley, CA (US)

(21) Appl. No.: 18/579,065

(22) PCT Filed: Aug. 24, 2022
(86) PCT No.: PCT/US2022/041410

§ 371 Date: Jan. 12, 2024

Related U.S. Application Data
(60) Provisional application No. 63/272,450, filed on Oct. 27, 2021.
(60) Provisional application No. 63/236,988, filed on Aug. 25, 2021.

(51) Int. Cl. A61K 47/68; A61P 35/00

(52) U.S. Cl. CPC A61K 47/68033; A61K 47/6851; A61P 35/00

(57) ABSTRACT

This disclosure provides methods of using antibody-drug-conjugates of formula (I). Specifically, the disclosure provides methods of reducing target-mediated cross-reactivity by using the antibody-drug-conjugates (ADCs) of formula (I). The disclosure also includes methods of using such conjugates in a variety of therapeutic indications, as well as methods of production of such conjugates.

### CROSS-REFERENCE TO RELATED APPLICATIONS

This application claims the benefit of priority to U.S. Provisional Application No. 63/236,988, filed Aug. 25, 2021, and U.S. Provisional Application No. 63/272,450, filed Oct. 27, 2021, the disclosures of which are incorporated herein by reference.

### INTRODUCTION

The field of protein-small molecule therapeutic conjugates has advanced greatly, providing a number of clinically beneficial drugs with the promise of providing more in the years to come. Protein-conjugate therapeutics can provide several advantages, due to, for example, specificity, multiplicity of functions, and relatively low off-target activity, resulting in fewer side effects. Chemical modification of proteins may extend these advantages by rendering them more potent, stable, or multimodal.

A number of standard chemical transformations are commonly used to create and manipulate post-translational modifications on proteins. There are a number of methods where one is able to selectively modify the side chains of certain amino acids. For example, carboxylic acid side chains (aspartate and glutamate) may be targeted by initial activation with a water-soluble carbodiimide reagent and subsequent reaction with an amine. Similarly, lysine can be targeted through the use of activated esters or isothiocyanates, and cysteine thiols can be targeted with maleimides and α-halo-carbonyls.

One significant obstacle to the creation of a chemically altered protein therapeutic or reagent is the production of the protein in a biologically active, homogenous form. Conjugation of a drug or detectable label to a polypeptide can be difficult to control, resulting in a heterogeneous mixture of conjugates that differ in the number of drug molecules attached and in the position of chemical conjugation. In some instances, it may be desirable to control the site of conjugation and/or the drug or detectable label conjugated to the polypeptide using the tools of synthetic organic chemistry to direct the precise and selective formation of chemical bonds on a polypeptide.

Tumor Associated Calcium Signal Transducer 2 (TACSTD2), also known as Trophoblast cell surface antigen 2 (Trop-2), is a transmembrane glycoprotein encoded by the TACSTD2 gene. TACSTD2 is an intracellular calcium signal transducer. TACSTD2 is differentially expressed in many cancers. Particularly, while TACSTD2 is expressed in many normal tissues, it is overexpressed in many cancers. Indeed, overexpression of TACSTD2 has prognostic value. As such, TACSTD2 is a suitable therapeutic target in patients with certain cancers, particularly, breast cancers. TACSTD2 on cancer cells can be targeted through antibodies, antibody fusion proteins, chemical inhibitors, nanoparticles, etc. For example, sacituzumab govitecan is an antibody-drug conjugate comprising an anti-TACSTD2 antibody. Sacituzumab govitecan is approved for treatment of patients with certain types of breast cancers.

Mucin-1 (also referred to as Mucin 1 or MUC1) is a member of the mucin family. Mucins are O-glycosylated proteins that play an essential role in forming protective mucous barriers on epithelial surfaces. MUC1 is expressed on the apical surface of epithelial cells that line the mucosal surfaces of many different tissues including lung, breast, stomach and pancreas. This protein is proteolytically cleaved into alpha and beta subunits that form a heterodimeric complex. The N-terminal alpha subunit functions in cell-adhesion and the C-terminal beta subunit is involved in cell signaling. Overexpression, aberrant intracellular localization, and changes in glycosylation of this protein have been associated with carcinomas.

NaPi2B (also referred to as Sodium-phosphate transport protein 2B) is a multitransmembrane, sodium-dependent phosphate transporter. While NaPi2B is expressed in many normal tissues, it is overexpressed in many cancers. Particularly, NaPi2B is expressed in human lung, ovarian, and thyroid cancers. Indeed, overexpression of NaPi2B has prognostic value. As a member of the SLC34 solute carrier protein family, it is responsible for transcellular inorganic phosphate absorption and maintenance of phosphate homeostasis and has been associated with cell differentiation and tumorigenesis. NAPi2B on cancer cells can be targeted through antibodies, antibody fusion proteins, chemical inhibitors, nanoparticles, etc. For example, Lifastuzumab vedotin is an antibody-drug conjugate comprising an anti-NaPi2B antibody.

Nectin-4 belongs to the nectin family that has diverse physiological and pathological functions in humans. PVRL4 (poliovirus-receptor-like 4), is expressed specifically in the embryo and placenta. It was recently reported that Nectin-4 is overexpressed in several human cancers, including lung, ovarian, and breast cancer. It was also demonstrated that a soluble form of Nectin-4 has a potential as a diagnostic marker for several cancers. Furthermore, a few clinical studies have shown that there were significant inverse correlations between tumor Nectin-4 expression and the prognosis of the patients with lung and breast cancers. Enfortumab vedotin is an antibody-drug conjugate comprising an anti-Nectin-4 antibody.

### SUMMARY

An antibody-drug-conjugate (ADC) generally includes an antibody linked to a cytotoxic small molecule and are targeted at non-healthy cells. As a target antigen is sometimes expressed on both the non-healthy cell as well as a healthy cell, in-vivo, the payload (linker-drug or linker-small molecule) may be offloaded on either cells. In this case, the ADC may target the off-target or healthy cells that express the same antigen as the non-healthy cells. This may result in what is called cross-reactivity that can be clinically detected. For example, administration of an ADC to a subject may elicit toxicity associated with target-mediated cross-reactivity of the ADC. The toxicity may imply a limited dosage, irrespective of the specificity or the efficacy of the ADC itself. In some instances, therefore, it may be desirable to reduce the toxicity caused by the cross-reactivity of the ADC with healthy cells expressing the target antigen(s).

The present disclosure provides a method of reducing toxicity in a subject, by administering an antibody-drug-conjugates (ADC) of formula (I) to the subject, wherein, the ADC of formula (I) is:

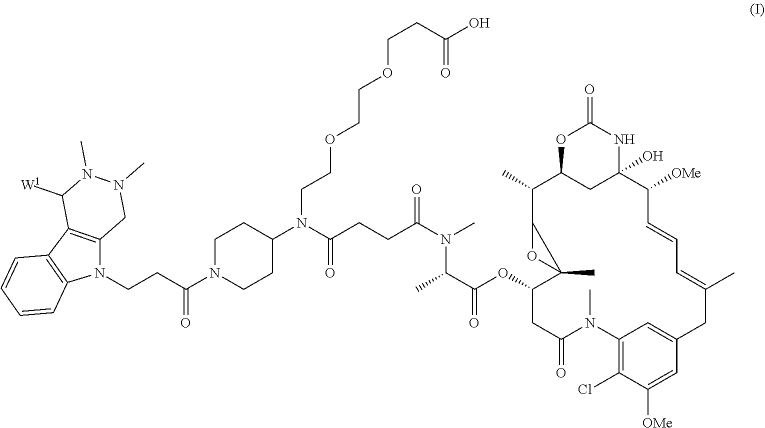

wherein the toxicity is associated with target-mediated cross-reactivity of the ADC when the ADC is administered to the subject.

The disclosure also encompasses methods of production of such conjugates, as well as methods of using the conjugates.

The disclosure encompasses the antibody in Formula (I) to target an antigen selected from the group consisting of nectin-4, TACSTD2, EGFR, ERBB3, glycoprotein non-metastatic melanoma protein B (GPNMB), SLC39A6 (LIV-1), SLITRK6, GUCY2C, MUC1, NaPi2B, and cadherin 3.

In some embodiments, the antibody of the ADC of Formula(I) targets any one of nectin-4, TACSTD2, NaPi2B or Muc-1 for treating a cell proliferative disorder in the subject.

In some embodiments, the antibody-drug-conjugate of Formula(I) targeting TACSTD2 comprises the sequence:

X¹(fGly′)X²Z²⁰X³Z²⁰
wherein
Z²⁰ is either a proline or alanine residue;
Z³⁰ is a basic amino acid or an aliphatic amino acid;
X¹ may be present or absent and, when present, can be any amino acid, with the proviso that when the sequence is at the N-terminus of the antibody, X¹ is present; and
X² and X³ are each independently any amino acid.

In some embodiments, the antibody in the ADC of Formula (I) is a IgG1 antibody. In some embodiments, the antibody in the ADC of Formula (I) is a kappa antibody.

In some aspects, the antibody in the ADC of Formula (I) comprises a fGly′ residue, wherein fGly′ is an amino acid of the antibody coupled at W¹.

In some aspects, the antibody in the ADC of Formula (I) comprises a fGly′ that is positioned at or near a C-terminus of a heavy chain constant region of the antibody.

In some aspects, the antibody in the ADC of Formula (I) comprises a fGly′ residue positioned in a light chain constant region of the antibody.

In some aspects, the antibody in the ADC of Formula (I) comprises a fGly′ residue positioned in a heavy chain CH1 region of the antibody.

In some aspects, the antibody in the ADC of Formula (I) comprises a fGly′ residue positioned in a heavy chain CH2 region of the antibody.

In some aspects, the antibody in the ADC of Formula (I) comprises a fGly′ residue positioned in a heavy chain CH3 region of the antibody.

In some embodiments, the antibody-drug-conjugate of Formula(I) targets and binds to TACSTD2 antigen, and competes for binding to the TACSTD2 antigen with an antibody comprising:
a variable heavy chain (VH) polypeptide comprising:
   a V_{H} CDR1 comprising the amino acid sequence NYNMN (SEQ ID NO: 1),
   a V_{H} CDR2 comprising the amino acid sequence WINTYTGEPTYTDDFKG (SEQ ID NO: 2), and
   a V_{H} CDR3 comprising the amino acid sequence GGFGSSYWYFDV (SEQ ID NO: 3); and
a variable light chain (V_{L}) polypeptide comprising
   a V_{L} CDR1 comprising the amino acid sequence KASQDVSIAVA (SEQ ID NO: 4),
   a V_{L} CDR2 comprising the amino acid sequence SASYRYT (SEQ ID NO: 5), and
   a V_{L} CDR3 comprising the amino acid sequence QQHYITPLT (SEQ ID NO: 6).

In some instances, the anti-TACSTD2 antibody comprises:
a V_{H} CDR1 comprising the amino acid sequence NYNMN (SEQ ID NO: 1),
a V_{H} CDR2 comprising the amino acid sequence WINTYTGEPTYTDDFKG (SEQ ID NO: 2), and
a V_{H} CDR3 comprising the amino acid sequence GGFGSSYWYFDV (SEQ ID NO: 3); and
a variable light chain (V_{L}) polypeptide comprising
a V_{L} CDR1 comprising the amino acid sequence KASQDVSIAVA (SEQ ID NO: 4),
a V_{L} CDR2 comprising the amino acid sequence SASYRYT (SEQ ID NO: 5), and
   a V_{L} CDR3 comprising the amino acid sequence QQHYITPLT (SEQ ID NO: 6).

In some aspects, the antibody-drug-conjugate of Formula(I) targets and binds to TACSTD2 antigen, wherein the antibody comprises:
a variable heavy chain (VH) polypeptide comprising an amino acid sequence having 70% or greater identity to the amino acid sequence set forth in SEQ ID NO: 7; and
a variable light chain (VL) polypeptide comprising an amino acid sequence having 70% or greater identity to the amino acid sequence set forth in SEQ ID NO: 8.

In other embodiments, the antibody-drug-conjugate of Formula(I) targets and binds to Muc-1 antigen, wherein the antibody comprises:
a variable heavy chain (VH) chain comprising heavy chain CDRs1-3 (HCDRs1-3) of a V_{H} chain having the sequence:

(SEQ ID NO: 9)
EVQLVQSGAEVKKPGATVKISCKVSGYTFTDHTMHWIKQRPGKGLE
WMGYFYPRDDSTNYNEKFKGRVTLTADKSTDTAYMELSSLRSEDTAVYY
CARGLRYALDYWGQGTLVTVSS;

and
a variable light chain (VL) chain comprising light chain CDRs1-3 (LCDRs1-3) of a V_{L} chain having the sequence:

(SEQ ID NO: 7)
EIVLTQSPATLSLSPGERATLSCRASSSVSSSYLYWYQQKPQAPRLWIY
GTSNLASGVPARFSGSGSGTDYTLTISSLEPEDAAVYYCHQYAWSPPTF
GQGTKLEIK;
(SEQ ID NO: 1)
EIVLTQSPATLSLSPGERATLSCRASSSVGSSNLYWYQQKPGQAPRLWI
YRSTKLASGVPARFSGSGSGTDYTLTISSLEPEDAAVYYCHQYRWSPPT
FGQGTKLEIK;
or
(SEQ ID NO: 2)
EIVLTQSPATLSLSPGERATLSCRASSSVSSSYLYWYQQKPGQAPRLWI
IGTSNLASGVPARFSGSGSGTDYTLTISSLEPEDAAVYYCHQYSWSPPT
FGQGTKLEIK.

In other embodiments, the antibody-drug-conjugate of Formula(I) targets and binds to Muc-1 antigen, wherein the antibody comprises:
the HCDR1 comprises the amino acid sequence DHTMH (SEQ ID NO: 10);
the HCDR2 comprises the amino acid sequence YFYPRDDSTNYNEKFKG (SEQ ID NO: 11);
the HCDR3 comprises the amino acid sequence GLRYALDY (SEQ ID NO: 5);
the LCDR1 comprises the amino acid sequence RASSSVSSSYLY (SEQ ID NO: 6);
the LCDR2 comprises the amino acid sequence GTSNLAS (SEQ ID NO: 12); and the LCDR3 comprises the amino acid sequence HQYAWSPPT (SEQ ID NO: 13), as per Kabat definition.

In some embodiments, the antibody-drug-conjugate of Formula(I) targets and binds to Muc-1 antigen, wherein the antibody comprises:
the HCDR1 comprises the amino acid sequence DHTMH (SEQ ID NO: 10);
the HCDR2 comprises the amino acid sequence YFYPRDDSTNYNEKFKG (SEQ ID NO: 11);
the HCDR3 comprises the amino acid sequence GLRYALDY (SEQ ID NO: 5);
the LCDR1 comprises the amino acid sequence RASSSVGSSNLY (SEQ ID NO: 14);
the LCDR2 comprises the amino acid sequence RSTKLAS (SEQ ID NO: 15); and the LCDR3 comprises the amino acid sequence HQYRWSPPT (SEQ ID NO: 16), as per Kabat definition.

In some embodiments, the antibody-drug-conjugate of Formula(I) targets and binds to Muc-1 antigen, wherein the antibody comprises:
the HCDR1 comprises the amino acid sequence DHTMH (SEQ ID NO: 10);
the HCDR2 comprises the amino acid sequence YFYPRDDSTNYNEKFKG (SEQ ID NO: 11);
the HCDR3 comprises the amino acid sequence GLRYALDY (SEQ ID NO: 5);
the LCDR1 comprises the amino acid sequence RASSSVSSSYLY (SEQ ID NO: 6);
the LCDR2 comprises the amino acid sequence GTSNLAS (SEQ ID NO: 12); and the LCDR3 comprises the amino acid sequence HQYSWSPPT (SEQ ID NO: 17), as per Kabat definition.

In some embodiments, the antibody-drug-conjugate of Formula(I) targets and binds to Muc-1 antigen, wherein the antibody comprises:
a variable heavy chain (V_{H}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO: 9; and
a variable light chain (V_{L}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO: 7.

In some embodiments, the antibody-drug-conjugate of Formula(I) targets and binds to Muc-1 antigen, wherein the antibody comprises:
a variable heavy chain (V_{H}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO: 9; and a variable light chain (V_{L}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO: 1.

In some embodiments, the antibody-drug-conjugate of Formula(I) targets and binds to Muc-1 antigen, wherein the antibody comprises:
a variable heavy chain (V_{H}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO: 9; and
a variable light chain (V_{L}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO: 2.

In some aspects, the antibody-drug conjugate (ADC) of formula (I) is administered to the subject parenterally or non-parenterally.

In some aspects, the antibody in the ADC of Formula (I) is a monoclonal antibody.

In some aspects, the antibody in the ADC of Formula (I) is a humanized antibody.

In some aspects, the drug in the ADC of Formula (I) is an anti-cancer drug. In some embodiments, the anti-cancer drug is a maytansinoid.

In some aspects, the antibody in the ADC of Formula (I) attached to a target antigen expressed in the skin or mucosal epithelium of the subject. In some embodiments, the target antigen is expressed on vital organs of the subject. The vital organ of the subject is an organ that is essential for survival of a living subject. For e.g., the vital organ can be any of brain, heart, lung, liver, kidney or spleen. In some embodiments, the method of reducing toxicity by administering the antibody-drug-conjugate of Formula (I) in a subject is directed to a subject suffering from a cell proliferative disorder.

### BRIEF DESCRIPTION OF THE DRAWINGS

FIG. 1, shows in-vitro toxicity assay comparing Nectin-4 targeting ADCs.

FIG. 2 shows comparison of clinical observations in rats when dosed with anti-nectin-4-vedotin conjugate vs. anti-nectin-4 RED-106 conjugates.

FIGS. 3A-3D shows toxicokinetic analysis of rat plasma samples from nectin-4 ADC repeat dose toxicity study confirms dosing levels and shows improved in vivo stability of the RED-106 conjugate relative to the vedotin conjugate.

FIGS. 4A-4D shows comparison of potency when TACSTD2 targeting conjugates according to Formula (I) are exposed to TACSTD2-expressing target cell lines compared to when Maytansine carrying TACSTD2 targeting ADCs are exposed to the target cell lines.

FIG. 5 shows in-vivo efficacy of TACSTD2-targeted ADCs against the lung xenograft model, NCI-H292.

FIG. 6 shows CAT-10⁻¹⁰⁶ DAR of 1.71 as determined by HIC.

FIG. 7 shows CAT-10⁻¹⁰⁶ is 98.7% monomeric as determined by analytical SEC.

FIG. 8A shows Nectin-4 CH1/CT-tagged RED-106 conjugate DAR of 3.49 as determined by analytical PLRP.

FIG. 8B shows Nectin-4 CH1/CT-tagged RED-106 conjugate is 97% monomeric as determined by analytical SEC.

FIG. 8C shows Nectin-4 vedotin conjugate DAR of 4.17 as determined by HIC.

FIG. 8D shows Nectin-4 vedotin conjugate is 96% monomeric as determined by SEC.

FIG. 9A depicts a site map showing possible modification sites for generation of an aldehyde tagged Ig polypeptide. The upper sequence is the amino acid sequence of the conserved region of an IgG1 light chain polypeptide (SEQ ID NO:163) and shows possible modification sites in an Ig light chain; the lower sequence is the amino acid sequence of the conserved region of an Ig heavy chain polypeptide (GenBank Accession No. AAG00909; SEQ ID NO://) and shows possible modification sites in an Ig heavy chain. The heavy and light chain numbering is based on the full-length heavy and light chains.

FIG. 9B depicts an alignment of immunoglobulin heavy chain constant regions for IgG1 (SEQ ID NO:47), IgG2 (SEQ ID NO:73), IgG3 (SEQ ID NO:92), IgG4 (SEQ ID NO: 112), and IgA (SEQ ID NO:129), showing modification sites at which aldehyde tags can be provided in an immunoglobulin heavy chain. The heavy and light chain numbering is based on the full heavy and light chains.

FIG. 9C depicts an alignment of immunoglobulin light chain constant regions (from top to bottom SEQ ID NOs:163, //, //, //, and 175), showing modification sites at which aldehyde tags can be provided in an immunoglobulin light chain.

### DEFINITIONS

The following terms have the following meanings unless otherwise indicated. Any undefined terms have their art recognized meanings.

“Alkyl” refers to monovalent saturated aliphatic hydrocarbyl groups having from 1 to 10 carbon atoms and such as 1 to 6 carbon atoms, or 1 to 5, or 1 to 4, or 1 to 3 carbon atoms. This term includes, by way of example, linear and branched hydrocarbyl groups such as methyl (CH3), ethyl (CH3CH2), n-propyl (CH3CH2CH2—), isopropyl ((CH3)2CH—), n-butyl (CH3CH2CH2CH2), isobutyl ((CH3)2CHCH2), sec-butyl ((CH3)(CH3CH2)CH—), t-butyl ((CH3)3C—), n-pentyl (CH3CH2CH2CH2CH2—), and neopentyl ((CH3)3CCH2—).

The term “substituted alkyl” refers to an alkyl group as defined herein wherein one or more carbon atoms in the alkyl chain (except the C1 carbon atom) have been optionally replaced with a heteroatom such as —O—, N—, S—, —S(O)n- (where n is 0 to 2), —NR— (where R is hydrogen or alkyl) and having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO2-alkyl, —SO2-aryl, SO2-heteroaryl, and —NraRb, wherein R′ and R″ may be the same or different and are chosen from hydrogen, optionally substituted alkyl, cycloalkyl, alkenyl, cycloalkenyl, alkynyl, aryl, heteroaryl and heterocyclic.

“Alkylene” refers to divalent aliphatic hydrocarbyl groups preferably having from 1 to 6 and more preferably 1 to 3 carbon atoms that are either straight-chained or branched, and which are optionally interrupted with one or more groups selected from —O—, —NR10—, NR10C(O)—, —C(O)NR10— and the like. This term includes, by way of example, methylene (CH2), ethylene (CH2CH2), n-propylene (CH2CH2CH2), iso-propylene (CH2CH(CH3)), (C(CH3)2CH2CH2), (C(CH3)2CH2C(O)), (C(CH3)2CH2C(O)NH), (CH(CH3)CH2—), and the like.

“Substituted alkylene” refers to an alkylene group having from 1 to 3 hydrogens replaced with substituents as described for carbons in the definition of “substituted” below.

The term “alkane” refers to alkyl group and alkylene group, as defined herein.

The term “alkylaminoalkyl,” “alkylaminoalkenyl,” and “alkylaminoalkynyl” refers to the groups R′NHR″—where R′ is alkyl group as defined herein and R″ is alkylene, alkenylene or alkynylene group as defined herein.

The term “alkaryl” or “aralkyl” refers to the groups -alkylene-aryl and substituted alkylene-aryl where alkylene, substituted alkylene and aryl are defined herein.

“Alkoxy” refers to the group —O-alkyl, wherein alkyl is as defined herein. Alkoxy includes, by way of example, methoxy, ethoxy, n-propoxy, isopropoxy, n-butoxy, t-butoxy, sec-butoxy, n-pentoxy, and the like. The term “alkoxy” also refers to the groups alkenyl-O—, cycloalkyl-O—, cycloalkenyl-O—, and alkynyl-O—, where alkenyl, cycloalkyl, cycloalkenyl, and alkynyl are as defined herein.

The term “substituted alkoxy” refers to the groups substituted alkyl-O—, substituted alkenyl-O—, substituted cycloalkyl-O—, substituted cycloalkenyl-O—, and substituted alkynyl-O— where substituted alkyl, substituted alkenyl, substituted cycloalkyl, substituted cycloalkenyl and substituted alkynyl are as defined herein.

The term “alkoxyamino” refers to the group —NH-alkoxy, wherein alkoxy is defined herein.

The term “haloalkoxy” refers to the groups alkyl-O— wherein one or more hydrogen atoms on the alkyl group have been substituted with a halo group and include, by way of examples, groups such as trifluoromethoxy, and the like.

The term “haloalkyl” refers to a substituted alkyl group as described above, wherein one or more hydrogen atoms on the alkyl group have been substituted with a halo group. Examples of such groups include, without limitation, fluoroalkyl groups, such as trifluoromethyl, difluoromethyl, trifluoroethyl and the like.

The term “alkylalkoxy” refers to the groups -alkylene-O-alkyl, alkylene-O-substituted alkyl, substituted alkylene-O-alkyl, and substituted alkylene-O-substituted alkyl wherein alkyl, substituted alkyl, alkylene and substituted alkylene are as defined herein.

The term “alkylthioalkoxy” refers to the group -alkylene-S-alkyl, alkylene-S-substituted alkyl, substituted alkylene-S-alkyl and substituted alkylene-S-substituted alkyl wherein alkyl, substituted alkyl, alkylene and substituted alkylene are as defined herein.

“Alkenyl” refers to straight chain or branched hydrocarbyl groups having from 2 to 6 carbon atoms and preferably 2 to 4 carbon atoms and having at least 1 and preferably from 1 to 2 sites of double bond unsaturation. This term includes, by way of example, bi vinyl, allyl, and but 3 en 1 yl. Included within this term are the cis and trans isomers or mixtures of these isomers.

The term “substituted alkenyl” refers to an alkenyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, SO-alkyl, —SO— substituted alkyl, SO-aryl, —SO-heteroaryl, —SO2-alkyl, —SO2-substituted alkyl, SO2-aryl and —SO2-heteroaryl.

“Alkynyl” refers to straight or branched monovalent hydrocarbyl groups having from 2 to 6 carbon atoms and preferably 2 to 3 carbon atoms and having at least 1 and preferably from 1 to 2 sites of triple bond unsaturation. Examples of such alkynyl groups include acetylenyl (C≡CH), and propargyl (CH2C≡CH).

The term “substituted alkynyl” refers to an alkynyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO— substituted alkyl, SO-aryl, —SO-heteroaryl, —SO2-alkyl, SO2-substituted alkyl, —SO2-aryl, and —SO2-heteroaryl.

“Alkynyloxy” refers to the group —O-alkynyl, wherein alkynyl is as defined herein. Alkynyloxy includes, by way of example, ethynyloxy, propynyloxy, and the like.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl C(O)—, heteroaryl-C(O)—, substituted heteroaryl-C(O)—, heterocyclyl-C(O)—, and substituted heterocyclyl-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. For example, acyl includes the “acetyl” group CH3C(O)—

“Acylamino” refers to the groups —NR20C(O)alkyl, —NR20C(O) substituted alkyl, N R20C(O)cycloalkyl, —NR20C(O) substituted cycloalkyl, —NR20C(O)cycloalkenyl, NR20C(O) substituted cycloalkenyl, —NR20C(O)alkenyl, —NR20C(O) substituted alkenyl, NR20C(O)alkynyl, —NR20C(O) substituted alkynyl, NR20C(O)aryl, NR20C(O) substituted aryl, NR20C(O)heteroaryl, NR20C(O) substituted heteroaryl, NR20C(O)heterocyclic, and NR20C(O) substituted heterocyclic, wherein R20 is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyl” or the term “aminoacyl” refers to the group C(O)NR21R22, wherein R21 and R22 independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R21 and R22 are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonylamino” refers to the group —NR21C(O)NR22R23 where R21, R22, and R23 are independently selected from hydrogen, alkyl, aryl or cycloalkyl, or where two R groups are joined to form a heterocyclyl group.

The term “alkoxycarbonylamino” refers to the group —NRC(O)OR where each R is independently hydrogen, alkyl, substituted alkyl, aryl, heteroaryl, or heterocyclyl wherein alkyl, substituted alkyl, aryl, heteroaryl, and heterocyclyl are as defined herein.

The term “acyloxy” refers to the groups alkyl-C(O)O—, substituted alkyl-C(O)O—, cycloalkyl-C(O)O—, substituted cycloalkyl-C(O)O—, aryl-C(O)O—, heteroaryl-C(O)O—, and heterocyclyl-C(O)O— wherein alkyl, substituted alkyl, cycloalkyl, substituted cycloalkyl, aryl, heteroaryl, and heterocyclyl are as defined herein.

“Aminosulfonyl” refers to the group —SO2NR21R22, wherein R21 and R22 independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic and where R21 and R22 are optionally joined together with the nitrogen bound thereto to form a heterocyclic or substituted heterocyclic group and alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Sulfonylamino” refers to the group —NR21SO2R22, wherein R21 and R22 independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R21 and R22 are optionally joined together with the atoms bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aryl” or “Ar” refers to a monovalent aromatic carbocyclic group of from 6 to 18 carbon atoms having a single ring (such as is present in a phenyl group) or a ring system having multiple condensed rings (examples of such aromatic ring systems include naphthyl, anthryl and indanyl) which condensed rings may or may not be aromatic, provided that the point of attachment is through an atom of an aromatic ring. This term includes, by way of example, phenyl and naphthyl. Unless otherwise constrained by the definition for the aryl substituent, such aryl groups can optionally be substituted with from 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO2-alkyl, —SO2-substituted alkyl, —SO2-aryl, —SO2-heteroaryl and trihalomethyl.

“Aryloxy” refers to the group —O-aryl, wherein aryl is as defined herein, including, by way of example, phenoxy, naphthoxy, and the like, including optionally substituted aryl groups as also defined herein.

“Amino” refers to the group —NH2.

The term “substituted amino” refers to the group —NRR where each R is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl, and heterocyclyl provided that at least one R is not hydrogen.

The term “azido” refers to the group —N3.

“Carboxyl,” “carboxy” or “carboxylate” refers to —CO2H or salts thereof.

“Carboxyl ester” or “carboxy ester” or the terms “carboxyalkyl” or “carboxylalkyl” refers to the groups C(O)O alkyl, C(O)O substituted alkyl, C(O)O alkenyl, C(O)O substituted alkenyl, C(O)O alkynyl, C(O)O substituted alkynyl, C(O)O aryl, C(O)O substituted aryl, C(O)O cycloalkyl, C(O)O substituted cycloalkyl, C(O)O cycloalkenyl, C(O)O substituted cycloalkenyl, C(O)O heteroaryl, C(O)O substituted heteroaryl, C(O)O heterocyclic, and C(O)O substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” or “carbonate” refers to the groups —O—C(O)O-alkyl, O C(O)O substituted alkyl, —O—C(O)O-alkenyl, —O—C(O)O-substituted alkenyl, —O—C(O)O-alkynyl, O C(O)O substituted alkynyl, —O—C(O)O-aryl, —O—C(O)O-substituted aryl, —O—C(O)O-cycloalkyl, O—C(O)O-substituted cycloalkyl, —O—C(O)O-cycloalkenyl, —O—C(O)O-substituted cycloalkenyl, O C(O)O-heteroaryl, —O—C(O)O-substituted heteroaryl, —O—C(O)O-heterocyclic, and O C(O)O substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” or “nitrile” refers to the group —CN.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclooctyl and the like. Such cycloalkyl groups include, by way of example, single ring structures such as cyclopropyl, cyclobutyl, cyclopentyl, cyclooctyl, and the like, or multiple ring structures such as adamantanyl, and the like.

The term “substituted cycloalkyl” refers to cycloalkyl groups having from 1 to 5 substituents, or from 1 to 3 substituents, selected from alkyl, substituted alkyl, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, SO-alkyl, —SO— substituted alkyl, —SO-aryl, —SO-heteroaryl, —SO2-alkyl, —SO2-substituted alkyl, SO2-aryl and —SO2-heteroaryl.

“Cycloalkenyl” refers to non-aromatic cyclic alkyl groups of from 3 to 10 carbon atoms having single or multiple rings and having at least one double bond and preferably from 1 to 2 double bonds.

The term “substituted cycloalkenyl” refers to cycloalkenyl groups having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, SO-alkyl, SO-substituted alkyl, —SO-aryl, —SO-heteroaryl, —SO2-alkyl, —SO2-substituted alkyl, SO2-aryl and —SO2-heteroaryl.

“Cycloalkynyl” refers to non-aromatic cycloalkyl groups of from 5 to 10 carbon atoms having single or multiple rings and having at least one triple bond.

“Cycloalkoxy” refers to —O-cycloalkyl.

“Cycloalkenyloxy” refers to —O-cycloalkenyl.

“Halo” or “halogen” refers to fluoro, chloro, bromo, and iodo.

“Hydroxy” or “hydroxyl” refers to the group —OH.

“Heteroaryl” refers to an aromatic group of from 1 to 15 carbon atoms, such as from 1 to 10 carbon atoms and 1 to 10 heteroatoms selected from the group consisting of oxygen, nitrogen, and sulfur within the ring. Such heteroaryl groups can have a single ring (such as, pyridinyl, imidazolyl or furyl) or multiple condensed rings in a ring system (for example as in groups such as, indolizinyl, quinolinyl, benzofuran, benzimidazolyl or benzothienyl), wherein at least one ring within the ring system is aromatic. To satisfy valence requirements, any heteroatoms in such heteroaryl rings may or may not be bonded to H or a substituent group, e.g., an alkyl group or other substituent as described herein. In certain embodiments, the nitrogen and/or sulfur ring atom(s) of the heteroaryl group are optionally oxidized to provide for the N-oxide (N→O), sulfinyl, or sulfonyl moieties. This term includes, by way of example, pyridinyl, pyrrolyl, indolyl, thiophenyl, and furanyl. Unless otherwise constrained by the definition for the heteroaryl substituent, such heteroaryl groups can be optionally substituted with 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, SO-heteroaryl, SO2-alkyl, —SO2-substituted alkyl, —SO2-aryl and —SO2-heteroaryl, and trihalomethyl.

The term “heteroaralkyl” refers to the groups -alkylene-heteroaryl where alkylene and heteroaryl are defined herein. This term includes, by way of example, pyridylmethyl, pyridylethyl, indolylmethyl, and the like.

“Heteroaryloxy” refers to —O-heteroaryl.

“Heterocycle,” “heterocyclic,” “heterocycloalkyl,” and “heterocyclyl” refer to a saturated or unsaturated group having a single ring or multiple condensed rings, including fused bridged and spiro ring systems, and having from 3 to 20 ring atoms, including 1 to 10 hetero atoms. These ring atoms are selected from nitrogen, sulfur, or oxygen, where, in fused ring systems, one or more of the rings can be cycloalkyl, aryl, or heteroaryl, provided that the point of attachment is through the non-aromatic ring. In certain embodiments, the nitrogen and/or sulfur atom(s) of the heterocyclic group are optionally oxidized to provide for the N-oxide, —S(O)—, or —SO2— moieties. To satisfy valence requirements, any heteroatoms in such heterocyclic rings may or may not be bonded to one or more H or one or more substituent group(s), e.g., an alkyl group or other substituent as described herein.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, and the like.

Unless otherwise constrained by the definition for the heterocyclic substituent, such heterocyclic groups can be optionally substituted with 1 to 5, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, SO-aryl, —SO-heteroaryl, SO2-alkyl, —SO2-substituted alkyl, —SO2-aryl, SO2-heteroaryl, and fused heterocycle.

“Heterocyclyloxy” refers to the group —O-heterocyclyl.

The term “heterocyclylthio” refers to the group heterocyclic-S—.

The term “heterocyclene” refers to the diradical group formed from a heterocycle, as defined herein.

The term “hydroxyamino” refers to the group —NHOH.

“Nitro” refers to the group —NO2.

“Oxo” refers to the atom (═O).

“Sulfonyl” refers to the group SO2-alkyl, SO2-substituted alkyl, SO2-alkenyl, SO2-substituted alkenyl, SO2-cycloalkyl, SO2-substituted cycloalkyl, SO2-cycloalkenyl, SO2-substituted cylcoalkenyl, SO2-aryl, SO2-substituted aryl, SO2-heteroaryl, SO2-substituted heteroaryl, SO2-heterocyclic, and SO2-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Sulfonyl includes, by way of example, methyl-SO2—, phenyl-SO2—, and 4-methylphenyl-SO2—.

“Sulfonyloxy” refers to the group —OSO2-alkyl, OSO2-substituted alkyl, OSO2-alkenyl, OSO2-substituted alkenyl, OSO2-cycloalkyl, OSO2-substituted cycloalkyl, OSO2-cycloalkenyl, OSO2-substituted cylcoalkenyl, OSO2-aryl, OSO2-substituted aryl, OSO2-heteroaryl, OSO2-substituted heteroaryl, OSO2-heterocyclic, and OSO2 substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

The term “aminocarbonyloxy” refers to the group OC(O)NRR where each R is independently hydrogen, alkyl, substituted alkyl, aryl, heteroaryl, or heterocyclic wherein alkyl, substituted alkyl, aryl, heteroaryl and heterocyclic are as defined herein.

“Thiol” refers to the group —SH.

“Thioxo” or the term “thioketo” refers to the atom (═S).

“Alkylthio” or the term “thioalkoxy” refers to the group —S-alkyl, wherein alkyl is as defined herein. In certain embodiments, sulfur may be oxidized to —S(O)—. The sulfoxide may exist as one or more stereoisomers.

The term “substituted thioalkoxy” refers to the group —S-substituted alkyl.

The term “thioaryloxy” refers to the group aryl-S— wherein the aryl group is as defined herein including optionally substituted aryl groups also defined herein.

The term “thioheteroaryloxy” refers to the group heteroaryl-S— wherein the heteroaryl group is as defined herein including optionally substituted aryl groups as also defined herein.

The term “thioheterocyclooxy” refers to the group heterocyclyl-S— wherein the heterocyclyl group is as defined herein including optionally substituted heterocyclyl groups as also defined herein.

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, ═NR70, ═N—OR70, ═N2 or ═S) on saturated carbon atoms in the specified group or radical are, unless otherwise specified, R60, halo, ═O, OR70, SR70, NR80R80, trihalomethyl, CN, OCN, SCN, NO, NO2, ═N2, N3, SO2R70, SO2O-M+, SO2OR70, OSO2R70, OSO2O-M+, OSO2OR70, P(O)(O—)2(M+)2, P(O)(OR70)O-M+, P(O)(OR70) 2, C(O)R70, C(S)R70, C(NR70)R70, C(O)O-M+, C(O)OR70, C(S)OR70, C(O)NR80R80, C(NR70)NR80R80, OC(O)R70, OC(S)R70, OC(O)O M+, OC(O)OR70, OC(S)OR70, NR70C(O)R70, NR70C(S)R70, NR70CO2-M+, NR70CO2R70, NR70C(S)OR70, NR70C(O)NR80R80, NR70C(NR70)R70 and NR70C(NR70)NR80R80, where R60 is selected from the group consisting of optionally substituted alkyl, cycloalkyl, heteroalkyl, heterocycloalkylalkyl, cycloalkylalkyl, aryl, arylalkyl, heteroaryl and heteroarylalkyl, each R70 is independently hydrogen or R60; each R80 is independently R70 or alternatively, two R80's, taken together with the nitrogen atom to which they are bonded, form a 5-, 6- or 7-membered heterocycloalkyl which may optionally include from 1 to 4 of the same or different additional heteroatoms selected from the group consisting of O, N and S, of which N may have —H or C1-C3 alkyl substitution; and each M+ is a counter ion with a net single positive charge. Each M+ may independently be, for example, an alkali ion, such as K+, Na+, Li+; an ammonium ion, such as +N(R60)4; or an alkaline earth ion, such as [Ca2+]0.5, [Mg2+]0.5, or [Ba2+]0.5 (“subscript 0.5 means that one of the counter ions for such divalent alkali earth ions can be an ionized form of a compound of the invention and the other a typical counter ion such as chloride, or two ionized compounds disclosed herein can serve as counter ions for such divalent alkali earth ions, or a doubly ionized compound of the invention can serve as the counter ion for such divalent alkali earth ions). As specific examples, NR80R80 is meant to include NH2, NH alkyl, N-pyrrolidinyl, N-piperazinyl, 4N-methyl-piperazin-1-yl and N-morpholinyl.

In addition to the disclosure herein, substituent groups for hydrogens on unsaturated carbon atoms in “substituted” alkene, alkyne, aryl and heteroaryl groups are, unless otherwise specified, R60, halo, O-M+, OR70, SR70, S-M+, NR80R80, trihalomethyl, CF3, CN, OCN, SCN, NO, NO2, N3, SO2R70, SO3-M+, SO3R70, OSO2R70, OSO2-M+, OSO3R70, PO3 2(M+)2, P(O)(OR70)O-M+, P(O)(OR70)2, C(O)R70, C(S)R70, C(NR70)R70, C02-M+, C02R70, C(S)OR70, C(O)NR80R80, C(NR70)NR80R80, OC(O)R70, OC(S)R70, OC02-M+, OSO3R70, OC(S)OR70, NR70C(O)R70, NR70C(S)R70, NR70CO2-M+, NR70CO2R70, NR70C(S)OR70, NR70C(O)NR80R80, NR70C(NR70)R70 and NR70C(NR70)NR80R80, where R60, R70, R80 and M+ are as previously defined, provided that in case of substituted alkene or alkyne, the substituents are not O-M+, OR70, SR70, or S-M+.

In addition to the groups disclosed with respect to the individual terms herein, substituent groups for hydrogens on nitrogen atoms in “substituted” heteroalkyl and cycloheteroalkyl groups are, unless otherwise specified, R60, O M+, OR70, SR70, S M+, NR80R80, trihalomethyl, CF3, CN, NO, NO2, S(O)2R70, S(O)2O M+, S(O)2OR70, OS(O)2R70, OS(O)2O M+, OS(O)2OR70, P(O)(O)2(M+)2, P(O)(OR70)O M+, P(O)(OR70)(OR70), C(O)R70, C(S)R70, C(NR70)R70, C(O)OR70, C(S)OR70, C(O)NR80R80, C(NR70)NR80R80, OC(O)R70, OC(S)R70, OC(O)OR70, OC(S)OR70, NR70C(O)R70, NR70C(S)R70, NR70C(O)OR70, NR70C(S)OR70, NR70C(O)NR80R80, NR70C(NR70)R70 and NR70C(NR70)NR80R80, where R60, R70, R80 and M+ are as previously defined.

In addition to the disclosure herein, in a certain embodiment, a group that is substituted has 1, 2, 3, or 4 substituents, 1, 2, or 3 substituents, 1 or 2 substituents, or 1 substituent.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups specifically contemplated herein are limited to substituted aryl-(substituted aryl)-substituted aryl.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

The term “pharmaceutically acceptable salt” means a salt which is acceptable for administration to a patient, such as a mammal (salts with counterions having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, formate, tartrate, besylate, mesylate, acetate, maleate, oxalate, and the like.

The term “salt thereof” means a compound formed when a proton of an acid is replaced by a cation, such as a metal cation or an organic cation and the like. Where applicable, the salt is a pharmaceutically acceptable salt, although this is not required for salts of intermediate compounds that are not intended for administration to a patient. By way of example, salts of the present compounds include those wherein the compound is protonated by an inorganic or organic acid to form a cation, with the conjugate base of the inorganic or organic acid as the anionic component of the salt.

“Solvate” refers to a complex formed by combination of solvent molecules with molecules or ions of the solute. The solvent can be an organic compound, an inorganic compound, or a mixture of both. Some examples of solvents include, but are not limited to, methanol, N,N-dimethylformamide, tetrahydrofuran, dimethylsulfoxide, and water. When the solvent is water, the solvate formed is a hydrate.

“Stereoisomer” and “stereoisomers” refer to compounds that have same atomic connectivity but different atomic arrangement in space. Stereoisomers include cis-trans isomers, E and Z isomers, enantiomers, and diastereomers.

“Tautomer” refers to alternate forms of a molecule that differ only in electronic bonding of atoms and/or in the position of a proton, such as enol-keto and imine-enamine tautomers, or the tautomeric forms of heteroaryl groups containing a —N═C(H)—NH— ring atom arrangement, such as pyrazoles, imidazoles, benzimidazoles, triazoles, and tetrazoles. A person of ordinary skill in the art would recognize that other tautomeric ring atom arrangements are possible.

It will be appreciated that the term “or a salt or solvate or stereoisomer thereof” is intended to include all permutations of salts, solvates and stereoisomers, such as a solvate of a pharmaceutically acceptable salt of a stereoisomer of subject compound.

“Pharmaceutically effective amount” and “therapeutically effective amount” refer to an amount of a compound sufficient to treat a specified disorder or disease or one or more of its symptoms and/or to prevent the occurrence of the disease or disorder. In reference to tumorigenic proliferative disorders, a pharmaceutically or therapeutically effective amount comprises an amount sufficient to, among other things, cause the tumor to shrink or decrease the growth rate of the tumor.

“Patient” refers to human and non-human subjects, especially mammalian subjects.

The term “treating” or “treatment” as used herein means the treating or treatment of a disease or medical condition in a patient, such as a mammal (particularly a human) that includes: (a) preventing the disease or medical condition from occurring, such as, prophylactic treatment of a subject; (b) ameliorating the disease or medical condition, such as, eliminating or causing regression of the disease or medical condition in a patient; (c) suppressing the disease or medical condition, for example by, slowing or arresting the development of the disease or medical condition in a patient; or (d) alleviating a symptom of the disease or medical condition in a patient.

The terms “polypeptide,” “peptide,” and “protein” are used interchangeably herein to refer to a polymeric form of amino acids of any length. Unless specifically indicated otherwise, “polypeptide,” “peptide,” and “protein” can include genetically coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, proteins which contain at least one N-terminal methionine residue (e.g., to facilitate production in a recombinant host cell); immunologically tagged proteins; and the like. In certain embodiments, a polypeptide is an antibody.

“Native amino acid sequence” or “parent amino acid sequence” are used interchangeably herein to refer to the amino acid sequence of a polypeptide prior to modification to include at least one modified amino acid residue.

The terms “amino acid analog,” “unnatural amino acid,” and the like may be used interchangeably, and include amino acid-like compounds that are similar in structure and/or overall shape to one or more amino acids commonly found in naturally occurring proteins (e.g., Ala or A, Cys or C, Asp or D, Glu or E, Phe or F, Gly or G, His or H, Ile or I, Lys or K, Leu or L, Met or M, Asn or N, Pro or P, Gln or Q, Arg or R, Ser or S, Thr or T, Val or V, Trp or W, Tyr or Y). Amino acid analogs also include natural amino acids with modified side chains or backbones. Amino acid analogs also include amino acid analogs with the same stereochemistry as in the naturally occurring D-form, as well as the L-form of amino acid analogs. In some instances, the amino acid analogs share backbone structures, and/or the side chain structures of one or more natural amino acids, with difference(s) being one or more modified groups in the molecule. Such modification may include, but is not limited to, substitution of an atom (such as N) for a related atom (such as S), addition of a group (such as methyl, or hydroxyl, etc.) or an atom (such as Cl or Br, etc.), deletion of a group, substitution of a covalent bond (single bond for double bond, etc.), or combinations thereof. For example, amino acid analogs may include α-hydroxy acids, and α-amino acids, and the like.

The terms “amino acid side chain” or “side chain of an amino acid” and the like may be used to refer to the substituent attached to the α-carbon of an amino acid residue, including natural amino acids, unnatural amino acids, and amino acid analogs. An amino acid side chain can also include an amino acid side chain as described in the context of the modified amino acids and/or conjugates described herein.

The term “carbohydrate” and the like may be used to refer to monomers units and/or polymers of monosaccharides, disaccharides, oligosaccharides, and polysaccharides. The term sugar may be used to refer to the smaller carbohydrates, such as monosaccharides, disaccharides. The term “carbohydrate derivative” includes compounds where one or more functional groups of a carbohydrate of interest are substituted (replaced by any convenient substituent), modified (converted to another group using any convenient chemistry) or absent (e.g., eliminated or replaced by H). A variety of carbohydrates and carbohydrate derivatives are available and may be adapted for use in the subject compounds and conjugates.

The term “antibody” is used in the broadest sense and includes monoclonal antibodies (including full length monoclonal antibodies), polyclonal antibodies, and multispecific antibodies (e.g., bispecific antibodies), humanized antibodies, single-chain antibodies (e.g., scFv), chimeric antibodies, antibody fragments (e.g., Fab fragments), and the like. An antibody is capable of binding a target antigen. (Janeway, C., Travers, P., Walport, M., Shlomchik (2001) Immuno Biology, 5th Ed., Garland Publishing, New York). A target antigen can have one or more binding sites, also called epitopes, recognized by complementarity determining regions (CDRs) formed by one or more variable regions of an antibody.

The term “natural antibody” refers to an antibody in which the heavy and light chains of the antibody have been made and paired by the immune system of a multi-cellular organism. Spleen, lymph nodes, bone marrow and serum are examples of tissues that produce natural antibodies. For example, the antibodies produced by the antibody producing cells isolated from a first animal immunized with an antigen are natural antibodies.

The term “humanized antibody” or “humanized immunoglobulin” refers to a non-human (e.g., mouse or rabbit) antibody containing one or more amino acids (in a framework region, a constant region or a CDR, for example) that have been substituted with a correspondingly positioned amino acid from a human antibody. In general, humanized antibodies produce a reduced immune response in a human host, as compared to a non-humanized version of the same antibody. Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska. Et al., PNAS 91:969-973 (1994)), and chain shuffling (U.S. Pat. No. 5,565,332). In certain embodiments, framework substitutions are identified by modeling of the interactions of the CDR and framework residues to identify framework residues important for antigen binding and sequence comparison to identify unusual framework residues at particular positions (see, e.g., U.S. Pat. No. 5,585,089; Riechmann et al., Nature 332:323 (1988)). Additional methods for humanizing antibodies contemplated for use in the present invention are described in U.S. Pat. Nos. 5,750,078; 5,502,167; 5,705,154; 5,770,403; 5,698,417; 5,693,493; 5,558,864; 4,935,496; and 4,816,567, and PCT publications WO 98/45331 and WO 98/45332. In particular embodiments, a subject rabbit antibody may be humanized according to the methods set forth in US20040086979 and US20050033031. Accordingly, the antibodies described above may be humanized using methods that are well known in the art.

The term “chimeric antibodies” refer to antibodies whose light and heavy chain genes have been constructed, typically by genetic engineering, from antibody variable and constant region genes belonging to different species. For example, the variable segments of the genes from a mouse monoclonal antibody may be joined to human constant segments, such as gamma 1 and gamma 3. An example of a therapeutic chimeric antibody is a hybrid protein composed of the variable or antigen-binding domain from a mouse antibody and the constant or effector domain from a human antibody, although domains from other mammalian species may be used.

An immunoglobulin polypeptide immunoglobulin light or heavy chain variable region is composed of a framework region (FR) interrupted by three hypervariable regions, also called “complementarity determining regions” or “CDRs”. The extent of the framework region and CDRs have been defined (see, “Sequences of Proteins of Immunological Interest,” E. Kabat et al., U.S. Department of Health and Human Services, 1991). The framework region of an antibody, that is the combined framework regions of the constituent light and heavy chains, serves to position and align the CDRs. The CDRs are primarily responsible for binding to an epitope of an antigen.

As used herein the term “immunoglobulin” refers to a protein consisting of one or more polypeptides substantially encoded by immunoglobulin genes. The recognized human immunoglobulin genes include the kappa, lambda, alpha (IgA1 and IgA2), gamma (IgG1, IgG2, IgG3, IgG4), delta, epsilon and mu constant region genes; and numerous immunoglobulin variable region genes. Full-length immunoglobulin light chains (about 25 kD or 214 amino acids) are encoded by a variable region gene at the N-terminus (about 110 amino acids) and a kappa or lambda constant region at the C-terminus. Full-length immunoglobulin heavy chains (about 50 kD or 446 amino acids) are encoded by a variable region gene at the N-terminus (about 116 amino acids) and one of the other aforementioned constant region genes at the C-terminus, e.g. gamma (encoding about 330 amino acids). In some embodiments, a subject antibody comprises full-length immunoglobulin heavy chain and a full-length immunoglobulin light chain.

Throughout the present disclosure, the numbering of the residues in an immunoglobulin heavy chain and in an immunoglobulin light chain is that as in Kabat et al., Sequences of Proteins of Immunological Interest, 5th Ed. Public Health Service, National Institutes of Health, Bethesda, Md. (1991), expressly incorporated herein by reference.

A “parent Ig polypeptide” is a polypeptide comprising an amino acid sequence which lacks an aldehyde-tagged constant region as described herein. The parent polypeptide may comprise a native sequence constant region, or may comprise a constant region with pre-existing amino acid sequence modifications (such as additions, deletions and/or substitutions).

In the context of an Ig polypeptide, the term “constant region” is well understood in the art, and refers to a C-terminal region of an Ig heavy chain, or an Ig light chain. An Ig heavy chain constant region includes CH1, CH2, and CH3 domains (and CH4 domains, where the heavy chain is a p or an e heavy chain). In a native Ig heavy chain, the CH1, CH2, CH3 (and, if present, CH4) domains begin immediately after (C-terminal to) the heavy chain variable (VH) region, and are each from about 100 amino acids to about 130 amino acids in length. In a native Ig light chain, the constant region begins begin immediately after (C-terminal to) the light chain variable (VL) region, and is about 100 amino acids to 120 amino acids in length.

As used herein, the term “CDR” or “complementarity determining region” is intended to mean the non-contiguous antigen combining sites found within the variable region of both heavy and light chain polypeptides. CDRs have been described by Kabat et al., J. Biol. Chem. 252:6609-6616 (1977); Kabat et al., U.S. Dept. of Health and Human Services, “Sequences of proteins of immunological interest” (1991); by Chothia et al., J. Mol. Biol. 196:901-917 (1987); and MacCallum et al., J. Mol. Biol. 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison.

TABLE 1
CDR Definitions

| | Kabat¹ | Chothia² | MacCallum³ |
|---|---|---|---|
| V_{H} CDR1 | 31-35 | 26-32 | 30-35 |
| V_{H} CDR2 | 50-65 | 53-55 | 47-58 |
| V_{H} CDR3 | 95-102 | 96-101 | 93-101 |
| V_{L} CDR1 | 24-34 | 26-32 | 30-36 |
| V_{L} CDR2 | 50-56 | 50-52 | 46-55 |
| V_{L} CDR3 | 89-97 | 91-96 | 89-96 |

By “genetically-encodable” as used in reference to an amino acid sequence of polypeptide, peptide or protein means that the amino acid sequence is composed of amino acid residues that are capable of production by transcription and translation of a nucleic acid encoding the amino acid sequence, where transcription and/or translation may occur in a cell or in a cell-free in vitro transcription/translation system.

The terms “control sequences” and “regulatory sequences” refer to DNA sequences that facilitate expression of an operably linked coding sequence in a particular expression system, e.g. mammalian cell, bacterial cell, cell-free synthesis, etc. The control sequences that are suitable for prokaryote systems, for example, include a promoter, optionally an operator sequence, and a ribosome binding site. Eukaryotic cell systems may utilize promoters, polyadenylation signals, and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate the initiation of translation. Generally, “operably linked” means that the DNA sequences being linked are contiguous, and, in the case of a secretory leader, contiguous and in reading frame. Linking is accomplished by ligation or through amplification reactions. Synthetic oligonucleotide adaptors or linkers may be used for linking sequences in accordance with conventional practice.

The term “expression cassette” as used herein refers to a segment of nucleic acid, usually DNA, that can be inserted into a nucleic acid (e.g., by use of restriction sites compatible with ligation into a construct of interest or by homologous recombination into a construct of interest or into a host cell genome). In general, the nucleic acid segment comprises a polynucleotide that encodes a polypeptide of interest, and the cassette and restriction sites are designed to facilitate insertion of the cassette in the proper reading frame for transcription and translation. Expression cassettes can also comprise elements that facilitate expression of a polynucleotide encoding a polypeptide of interest in a host cell, e.g., a mammalian host cell. These elements may include, but are not limited to: a promoter, a minimal promoter, an enhancer, a response element, a terminator sequence, a polyadenylation sequence, and the like.

As used herein the term “isolated” is meant to describe a compound of interest that is in an environment different from that in which the compound naturally occurs. “Isolated” is meant to include compounds that are within samples that are substantially enriched for the compound of interest and/or in which the compound of interest is partially or substantially purified.

As used herein, the term “substantially purified” refers to a compound that is removed from its natural environment and is at least 60% free, at least 75% free, at least 80% free, at least 85% free, at least 90% free, at least 95% free, at least 98% free, or more than 98% free, from other components with which it is naturally associated.

The term “physiological conditions” is meant to encompass those conditions compatible with living cells, e.g., predominantly aqueous conditions of a temperature, pH, salinity, etc. that are compatible with living cells.

By “reactive partner” is meant a molecule or molecular moiety that specifically reacts with another reactive partner to produce a reaction product. Exemplary reactive partners include a cysteine or serine of a sulfatase motif and Formylglycine Generating Enzyme (FGE), which react to form a reaction product of a converted aldehyde tag containing a formylglycine (fGly) in lieu of cysteine or serine in the motif. Other exemplary reactive partners include an aldehyde of an fGly residue of a converted aldehyde tag (e.g., a reactive aldehyde group) and an “aldehyde-reactive reactive partner,” which comprises an aldehyde-reactive group and a moiety of interest, and which reacts to form a reaction product of a polypeptide having the moiety of interest conjugated to the polypeptide through the fGly residue.

“N-terminus” refers to the terminal amino acid residue of a polypeptide having a free amine group, which amine group in non-N-terminus amino acid residues normally forms part of the covalent backbone of the polypeptide.

“C-terminus” refers to the terminal amino acid residue of a polypeptide having a free carboxyl group, which carboxyl group in non-C-terminus amino acid residues normally forms part of the covalent backbone of the polypeptide.

By “internal site” as used in referenced to a polypeptide or an amino acid sequence of a polypeptide means a region of the polypeptide that is not at the N-terminus or at the C-terminus.

By “ADC other than Formula (I)”, as used herein in reference to a comparable ADC, refers to an antibody-drug-conjugate, where the linker-payload is either structurally or functionally, or both, different from the ADC of Formula (I) as disclosed herein. In some instances, the ADC other than Formula (I), is not encompassed by Formula (I) of the present disclosure. For instance, an ADC other than Formula (I), can refer to an antibody linked to a payload (drug), where the payload is any one or more of monomethyl auristatin E (Vedotin), dolastatin 10, DXd (MAAA-1181a; MAAA-1181), SN-38 (e.g., Trodelvy), camptothecin, MMAE, and analogs thereof. For instance, an ADC other than Formula (I), can refer to an ADC having a linker with a different structure compared to Formula (I).

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed, to the extent that such combinations embrace subject matter that are, for example, compounds that are stable compounds (i.e., compounds that can be made, isolated, characterized, and tested for biological activity). In addition, all sub-combinations of the various embodiments and elements thereof (e.g., elements of the chemical groups listed in the embodiments describing such variables) are also specifically embraced by the present invention and are disclosed herein just as if each and every such sub-combination was individually and explicitly disclosed herein.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

Certain embodiments of the present disclosure provide a method of reducing toxicity by administering to a subject, an antibody-drug conjugate of Formula (I). Also provided herein are methods of improving efficacy and stability of a production of such conjugates, as well as methods of using the same. Embodiments of each are described in more detail in the sections below.

### Methods of Using Antibody-Drug Conjugates

The present disclosure provides a method of reducing toxicity caused by target-mediated cross-reactivity of a conjugate, e.g., an antibody-drug conjugate (ADC). Particularly, the present disclosure provides a method of reducing toxicity caused by target-mediated cross-reactivity with the ADC of Formula (I). By “conjugate” is meant an antibody covalently attached to a moiety of interest (e.g., a drug or active agent). For example, a maytansine conjugate includes a maytansine (e.g., a maytansine active agent moiety) covalently attached to an antibody. In certain embodiments, the antibody and the drug or active agent are bound to each other through one or more functional groups and covalent bonds. For example, the one or more functional groups and covalent bonds can include a linker as described herein.

The methods of the present invention include a method of reducing the target-mediated cross-reactivity of an ADC in a subject. When a subject is treated with an antibody-drug conjugate (ADC), the antibody targets and binds to a specific antigen expressed on the surface of cells. In some instances, the targeted antigen is expressed on both healthy cells and non-healthy cells of the subject. In such cases, the antibody of the antibody-drug conjugate (ADC), acts on both healthy and non-healthy cells as they both express the target antigen. This phenomenon is understood to be target-mediated cross-reactivity and as an implication of this, particularly, as a result of delivery of the drug of the ADC to healthy cells, the subject might show any or a combination of clinical indications or reactions. For example, clinical indications or reaction can include, but are not limited, to those as listed in Table 2 below.

In some embodiments, methods of the present invention include the antibody-drug conjugate (ADC) of Formula (I), that decreases or reduces toxicity caused by the cross-reactivity in a subject, compared to the toxicity caused by cross-reactivity when the subject is administered an antibody-drug-conjugate not of Formula(I).

By reducing toxicity is meant a reduction or decrease in one or more of the parameter(s) as described in Table 2. The parameters can be scored based on a clinical observations scoring system, with Parameter 3 being the most intense and 0 being the least intense. Each parameter may correspond to a body region or a functional, physiological or behavioral aspect in a subject. By reducing toxicity, an ADC of Formula (I), as disclosed herein, reduces or decreases the intensity of the reaction parameter in the subject based on the clinical score(s) for each body region or physiological or behavioral aspect of the subject. For example, administration of an ADC of Formula (I), in a subject suffering from a cell proliferative disorder can effectively reduce the appearance of “deep wounds” (clinically scored as 3 or the most intense reaction) in the fur/skin of the subject to a score 1 showing minimal erythema or edema. In particular, use of an anti-TACSTD2 antibody-drug-conjugate of Formula (I), as disclosed herein, resulted in 0 dermal observations (score 0 based on Table 2) when administered to a subject compared to the observations of skin rash, lesions and mucosa (score 2 based on Table 2) when an ADC other than Formula (I) targeting anti-TACSTD2 was administered to the same subject.

TABLE 2
Clinical Observation Scoring System

| Parameter | 0 | 1 | 2 | 3 |
|---|---|---|---|---|
| Activity | Bright and alert | Minor changes, | Reduced mobility, | Comatose |
| Level / | | Stereotypic | Inactive, | |
| Unprovoked | | behavior, chirping | Huddled in cage, | |
| Behavior | | | Lethargic | |
| Provoked | Inquisitive | Minor depression | Moderately reduced | Violent reactions, |
| Behavior | about | or exaggeration of | response, | Loud and |
| | environment | response; | Moderate | continuous |
| | | Burrowing or | vocalization, | vocalizations |
| | | hiding, but rouses | No exploration | |
| | | when touched. | when lid removed | |
| Locomotion / | Normal | Tail stiff/upright, | Teetering or | Inability to move, |
| Neurological | | Tail drags, Head | stumbling, | Paralysis, |
| | | tilt, | Back | Dragging limbs, |
| | | Circling, | hunched/abdomen | Severe/Prolonged |
| | | | tucked while | convulsions |
| | | | walking, Tremor | |
| Respiration | Normal | Mildly pronounced | Open mouth | Severely |
| | | or reduced chest | breathing, | pronounced or |
| | | movement | Moderately | reduced chest |
| | | | pronounced or | movement |
| | | | reduced chest | |
| | | | movement | |
| Posture | Normal | Head tucked down | Hunched | Prostrate |
| | | | back/tucked | |
| | | | abdomen | |
| Body | Normal | Spinal column | Noticeable | Missing anatomy, |
| Condition | | evident, | distended abdomen, | Skeletal structure |
| | | Mild edema | Moderate edema | extremely |
| | | Loose | Moderate loose | prominent, |
| | | skin/dehydration | skin/dehydration | Distended |
| | | | | abdomen, |
| | | | | Severe edema |
| Fur & Skin | Shiny, well | Signs of minimal | Rough, starry coat, | Deep wounds |
| | groomed coat. | lack of grooming, | Severe piloerection, | (severe fighting |
| | | Signs of mild hair | Moderate skin | lesions, |
| | | loss, | lesions, | Skin ulceration, |
| | | Inflamed skin, | Soiled anogenital | Freund's complete |
| | | Mild piloerection | area, | adjuvant ulcer) |
| | | | Anal prolapse | |
| Eyes | Normal | Mild porphyrin | Obvious porphyrin | N/A |
| | | staining around | staining around eyes | |
| | | eyes | or on paws | |
| Tumors or | Normal | Small (abscess or | Moderate abscess or | Large abscess or |
| Infections* | | tumor (non-cancer | tumor (non-cancer | tumor (non-cancer |
| *unrelated to | | studies) | studies) | studies) |
| disease | | | | |
| models | | | | |
| Body weight | >0 or <10% | 10-15% loss from | 15-20% loss from | >20% loss from |
| | loss from | baseline | baseline | baseline |
| | baseline | | | |

In some instances, the ADC other than Formula (I), as used herein, refers to an antibody-drug-conjugate, wherein the linker-payload is either structurally or functionally, or both, different from the ADC of Formula (I) as disclosed herein. In some instances, the ADC other than Formula (I), is not encompassed by Formula (I) of the present disclosure. For instance, ADC other than Formula (I), can refer to an antibody linked to a payload (drug), where the payload is any one or more of monomethyl auristatin E (vedotin), dolastatin 10, DXd (MAAA-1181a; MAAA-1181), SN-38 (e.g., Trodelvy), camptothecin, MMAE, and analogs thereof. For instance, an ADC other than Formula (I), can refer to an ADC having a linker with a different structure compared to Formula (I).

In some further instances, an ADC of Formula (I), reduces or decreases the number of clinical observations or reactions to the ADC. In some subjects, an ADC other than that of Formula (I) shows a combination of clinical parameters shown in Table 2. For instance, there might be clinical observations of a combination of a growth or appearance of a large abscess or tumor (unrelated to the underlying disorder being treated) and deep wounds in various parts of skin of the subject as a reaction to the ADC other than that of Formula (I) administered to the subject. In contrast, when the same subject is administered an ADC of Formula (I) targeting the same antigen, the observed clinical observations might be limited to only mild skin rashes.

In some embodiments, by administering a subject the antibody-drug-conjugate of Formula (I), target-mediated cross-reactivity is reduced in the subject by at least 1 fold, 2 fold, 3 fold, 4 fold, 5 fold, 6 fold, 7 fold, 8 fold, 9 fold, or 10 fold or higher.

In some embodiments, by administering a subject the antibody-drug-conjugate of Formula (I), the target mediated cross-reactivity is reduced in the subject by reducing the clinical observation score of a particular parameter (as in Table 2) from a score of 3 to a score of 0, a score of 2 to a score of 1, a score of 1 to a score of 0, a score of 3 to a score of 2, a score of 3 to a score of 1, or a score of 2 to a score of 0. In some embodiments, the clinical observations of a reduction in score is observed across multiple parameters listed in Table 2.

In some embodiments, by administering a subject the antibody-drug-conjugate of Formula (I), stability of the conjugate in vivo is increased as compared to when the subject is administered an antibody-drug-conjugate other than that of formula(I), and wherein the target antigen is the same.

In some embodiments, the stability of the antibody-drug-conjugate of Formula (I) in vivo is increased by at least a multiple of 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98 or 100 or more.

In some embodiments, the stability of the antibody-drug-conjugate of Formula (I), in vivo, is increased by at least 10%, 20%, 30%, 40%, 50%, 60%, 70%, 80%, 90%, 95%, 98%, 99%, or a 100% or more compared to the stability of an antibody-drug-conjugate other than that of Formula (I), and wherein both the ADCs, as compared herein, target the same antigen.

In some aspects, by administering a subject the antibody-drug-conjugate of Formula (I), the ADC exhibits improved efficacy across a range of doses, as compared to an antibody-drug-conjugate other than that of Formula(I).

Further provided herein are methods that include administering to a subject an effective amount of any of the conjugates of the present disclosure.

In certain aspects, provided are methods of delivering a drug to a target site in a subject, the method including administering to the subject a pharmaceutical composition including any of the conjugates of the present disclosure, where the administering is effective to deliver a therapeutically effective amount of the drug to the target site in the subject.

By “treatment” is meant that at least an amelioration of the symptoms associated with the condition afflicting the host is achieved, where amelioration is used in a broad sense to refer to at least a reduction in the magnitude of a parameter, e.g. symptom, associated with the condition being treated. As such, treatment also includes situations where the pathological condition, or at least symptoms associated therewith, are completely inhibited, e.g., prevented from happening, or stopped, e.g. terminated, such that the host no longer suffers from the condition, or at least the symptoms that characterize the condition. Thus treatment includes: (i) prevention, that is, reducing the risk of development of clinical symptoms, including causing the clinical symptoms not to develop, e.g., preventing disease progression to a harmful state; (ii) inhibition, that is, arresting the development or further development of clinical symptoms, e.g., mitigating or completely inhibiting an active disease; and/or (iii) relief, that is, causing the regression of clinical symptoms.

The subject to be treated can be one that is in need of therapy, where the host to be treated is one amenable to treatment using the parent drug. Accordingly, a variety of subjects may be amenable to treatment using the polypeptide-drug conjugates disclosed herein. Generally, such subjects are “mammals,” with humans being of interest. Other subjects can include domestic pets (e.g., dogs and cats), livestock (e.g., cows, pigs, goats, horses, and the like), rodents (e.g., mice, guinea pigs, and rats, e.g., as in animal models of disease), as well as non-human primates (e.g., chimpanzees, and monkeys).

The amount of polypeptide-drug conjugate administered can be initially determined based on guidance of a dose and/or dosage regimen of the parent drug. In general, the polypeptide-drug conjugates can provide for targeted delivery and/or enhanced serum half-life of the bound drug, thus providing for at least one of reduced dose or reduced administrations in a dosage regimen. Thus, the polypeptide-drug conjugates can provide for reduced dose and/or reduced administration in a dosage regimen relative to the parent drug prior to being conjugated in an polypeptide-drug conjugate of the present disclosure.

Furthermore, as noted above, because the polypeptide-drug conjugates can provide for controlled stoichiometry of drug delivery, dosages of polypeptide-drug conjugates can be calculated based on the number of drug molecules provided on a per polypeptide-drug conjugate basis.

In some embodiments, multiple doses of a polypeptide-drug conjugate are administered. The frequency of administration of a polypeptide-drug conjugate can vary depending on any of a variety of factors, e.g., severity of the symptoms, condition of the subject, etc. For example, in some embodiments, a polypeptide-drug conjugate is administered once per month, twice per month, three times per month, every other week, once per week (qwk), twice per week, three times per week, four times per week, five times per week, six times per week, every other day, daily (qd/od), twice a day (bds/bid), or three times a day (tds/tid), etc.

### Methods of Treating Cancer

The present disclosure provides methods that include delivering a conjugate of the present disclosure to an individual having a cancer. The methods are useful for treating a wide variety of cancers, including carcinomas, sarcomas, leukemias, and lymphomas. In the context of cancer, the term “treating” includes one or more (e.g., each) of: reducing growth of a solid tumor, inhibiting replication of cancer cells, reducing overall tumor burden, and ameliorating one or more symptoms associated with a cancer.

Carcinomas that can be treated using a subject method include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma, etc.

Sarcomas that can be treated using a subject method include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

Other solid tumors that can be treated using a subject method include, but are not limited to, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

Leukemias that can be treated using a subject method include, but are not limited to, a) chronic myeloproliferative syndromes (neoplastic disorders of multipotential hematopoietic stem cells); b) acute myelogenous leukemias (neoplastic transformation of a multipotential hematopoietic stem cell or a hematopoietic cell of restricted lineage potential; c) chronic lymphocytic leukemias (CLL; clonal proliferation of immunologically immature and functionally incompetent small lymphocytes), including B-cell CLL, T-cell CLL prolymphocytic leukemia, and hairy cell leukemia; and d) acute lymphoblastic leukemias (characterized by accumulation of lymphoblasts). Lymphomas that can be treated using a subject method include, but are not limited to, B-cell lymphomas (e.g., Burkitt's lymphoma); Hodgkin's lymphoma; non-Hodgkin's B cell lymphoma; and the like.

In certain aspects, provided are methods of treating cancer in a subject, such methods including administering to the subject a therapeutically effective amount of a pharmaceutical composition including any of the conjugates of the present disclosure, where the administering is effective to treat cancer in the subject. In some embodiments, the cancer is a hematologic malignancy. Hematologic malignancies of interest include, but are not limited to, hematologic malignancies characterized by malignant B cells. Non-limiting examples of hematologic malignancies characterized by malignant B cells include leukemias (e.g., chronic lymphocytic leukemia (CLL)) and lymphomas (e.g., Non-Hodgkin lymphoma (NHL)). When the lymphoma is NHL, in certain aspects, the NHL is relapsed and/or refractory Non-Hodgkin lymphoma.

### Combination Therapy

In some embodiments, a subject method of treating a malignancy involves administering a subject conjugate and one or more additional therapeutic agents. Suitable additional therapeutic agents include, but are not limited to, a cancer chemotherapeutic agent (as described above).

In some cases, the additional therapeutic agent is an immunomodulatory therapeutic agent, such as checkpoint inhibitor or an interleukin. An immune checkpoint inhibitor inhibits the function of an immune inhibitory checkpoint molecule, such as a protein. An immune checkpoint inhibitor can be an antibody that specifically binds to an immune checkpoint protein. Various immune checkpoint inhibitors are known. Immune checkpoint inhibitors include, but are not limited to, peptides, antibodies, nucleic acid molecules, and small molecules.

Any suitable checkpoint inhibitor could be used in the methods disclosed herein. Examples of inhibitory checkpoint molecules include A2AR, B7-H3, B7-H4, BTLA, CTLA-4, CD277, IDO, KIR, PD-1, LAG-3, TIM-3, TIGIT and VISTA.

In some embodiments, an immune checkpoint inhibitor inhibits PD-1 signaling, for example, via inhibiting PD-1 or PD-L1. In some embodiments, an immune checkpoint inhibitor that inhibits PD-1 signaling is an anti-PD-1 antibody. In some embodiments, an anti-PD-1 antibody is nivolumab, pembrolizumab, atezolizumab, durvalumab, or avelumab. In some embodiments, an immune checkpoint inhibitor that inhibit PD-L1 includes, for example, AMP-244, MEDI-4736, MPDL328 OA, and MIH1.

In some embodiments, an immune checkpoint inhibitor is an inhibitor of CTLA-4, such as an antibody that targets CTLA-4, for example, ipilimumab.

In some embodiments, a checkpoint inhibitor targets CD366, which is a transmembrane protein also known as T cell immunoglobulin and mucin domain containing protein-3 (TIM-3).

Additional examples and certain aspects of immune checkpoint inhibitors are described by Hui (2019), Immune checkpoint inhibitors, J. Cell Biol., Vol. 218 No. 3 740-741, which is incorporated herein by reference in its entirety.

### Antibody-Drug Conjugates of Formula (I)

In certain embodiments, the conjugate is an antibody-drug conjugate (ADC), which includes an antibody conjugated to a drug or active agent through a linker. In certain embodiments, the conjugate is a maytansine conjugate, where an antibody is conjugated to a maytansine or a maytansine active agent moiety. “Maytansine,” “maytansine moiety,” “maytansine active agent moiety,” and “maytansinoid” refer to a maytansine and analogs and derivatives thereof, and pharmaceutically active maytansine moieties and/or portions thereof. A maytansine conjugated to the antibody can be any of a variety of maytansinoid moieties such as, but not limited to, maytansine and analogs and derivatives thereof as described herein, such as but not limited to deacyl maytansine.

The drug or active agent can be conjugated to the antibody at any desired site of the antibody. Thus, the present disclosure provides, for example, an antibody having a drug or active agent conjugated at a site at or near the C-terminus of the antibody. Other examples include an antibody having a drug or active agent conjugated at a position at or near the N-terminus of the antibody. Examples also include an antibody having a drug or active agent conjugated at a position between the C-terminus and the N-terminus of the antibody (e.g., at an internal site of the antibody). Combinations of the above are also possible where the antibody is conjugated to two or more drugs or active agents.

In certain embodiments, a conjugate of the present disclosure includes a maytansine conjugated to an amino acid residue of an antibody at the α-carbon of an amino acid residue. Stated another way, a maytansine conjugate includes an antibody where the side chain of one or more amino acid residues in the antibody have been modified and attached to a maytansine (e.g., attached to a maytansine through a linker as described herein). For example, a maytansine conjugate includes an antibody where the α-carbon of one or more amino acid residues in the antibody has been modified and attached to a maytansine (e.g., attached to a maytansine through a linker as described herein).

Embodiments of the present disclosure include conjugates where an antibody is conjugated to one or more moieties (e.g., drug or active agent), such as 2 moieties, 3 moieties, 4 moieties, 5 moieties, 6 moieties, 7 moieties, 8 moieties, 9 moieties, or 10 or more moieties. The moieties (e.g., drug or active agent) may be conjugated to the antibody at one or more sites in the antibody. For example, one or more moieties may be conjugated to a single amino acid residue of the antibody. In some cases, one moiety is conjugated to an amino acid residue of the antibody. In other embodiments, two moieties may be conjugated to the same amino acid residue of the antibody. In other embodiments, a first moiety is conjugated to a first amino acid residue of the antibody and a second moiety is conjugated to a second amino acid residue of the antibody. Combinations of the above are also possible, for example where an antibody is conjugated to a first moiety at a first amino acid residue and conjugated to two other moieties at a second amino acid residue. Other combinations are also possible, such as, but not limited to, an antibody conjugated to first and second moieties at a first amino acid residue and conjugated to third and fourth moieties at a second amino acid residue, etc.

In certain embodiments, the conjugates have an average drug-to-antibody ratio (DAR) (molar ratio) in the range of from 0.1 to 10, or from 0.5 to 10, or from 1 to 10, such as from 1 to 9, or from 1 to 8, or from 1 to 7, or from 1 to 6, or from 1 to 5, or from 1 to 4, or from 1 to 3, or from 1 to 2. In certain embodiments, the conjugates have an average DAR from 1 to 2, such as 1, 1.1, 1.2, 1.3, 1.4, 1.5, 1.6, 1.7, 1.8, 1.9 or 2. In certain embodiments, the conjugates have an average DAR of 1.5 to 2.5. In certain embodiments, the conjugates have an average DAR of 1.5 to 2. By average is meant the arithmetic mean.

The one or more amino acid residues of the antibody that are conjugated to the one or more moieties may be naturally occurring amino acids, unnatural amino acids, or combinations thereof. For instance, the conjugate may include a moiety (e.g., drug or active agent) conjugated to a naturally occurring amino acid residue of the antibody. In other instances, the conjugate may include a moiety conjugated to an unnatural amino acid residue of the antibody. One or more moieties may be conjugated to the antibody at a single natural or unnatural amino acid residue as described above. One or more natural or unnatural amino acid residues in the antibody may be conjugated to the moiety or moieties as described herein. For example, two (or more) amino acid residues (e.g., natural or unnatural amino acid residues) in the antibody may each be conjugated to one or two moieties, such that multiple sites in the antibody are conjugated to the moieties of interest.

In certain embodiments, the antibody and the drug or active agent are conjugated through a coupling moiety. For example, the antibody and the drug or active agent may each be bound (e.g., covalently bonded) to the coupling moiety, thus indirectly binding the antibody and the drug or active agent (e.g., maytansine) together through the coupling moiety. In some cases, the coupling moiety includes a hydrazinyl-indolyl or a hydrazinyl-pyrrolo-pyridinyl compound, or a derivative of a hydrazinyl-indolyl or a hydrazinyl-pyrrolo-pyridinyl compound. For instance, a general scheme for coupling a drug or active agent (e.g., a maytansine) to an antibody through a hydrazinyl-indolyl or a hydrazinyl-pyrrolo-pyridinyl coupling moiety is shown in the general reaction scheme below. Hydrazinyl-indolyl and hydrazinyl-pyrrolo-pyridinyl coupling moiety are also referred to herein as a 42ydrazine-iso-Pictet-Spengler (HIPS) coupling moiety and an aza-hydrazino-iso-Pictet-Spengler (azaHIPS) coupling moiety, respectively.

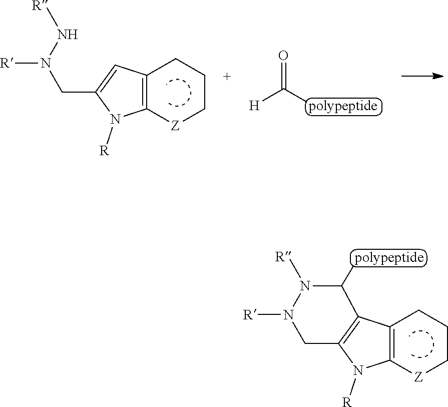

In the reaction scheme above, R is the drug or active agent (e.g., maytansine) that is conjugated to the antibody. As shown in the reaction scheme above, an antibody that includes a 2-formylglycine residue (fGly) is reacted with a drug (e.g., maytansine) that has been modified to include a coupling moiety (e.g., a hydrazinyl-indolyl or a hydrazinyl-pyrrolo-pyridinyl coupling moiety) to produce an antibody conjugate attached to the coupling moiety, thus attaching the maytansine to the antibody through the coupling moiety.

As described herein, the moiety can be any of a variety of moieties such as, but not limited to, a chemical entity, such as a drug or an active agent (e.g., a maytansinoid). R′ and R″ may each independently be any desired substituent, such as, but not limited to, hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. Z may be CR¹¹, NR¹², N, O or S, where R¹¹ and R¹² are each independently selected from any of the substituents described for R′ and R″ above.

Other hydrazinyl-indolyl or hydrazinyl-pyrrolo-pyridinyl coupling moieties are also possible, as shown in the conjugates and compounds described herein. For example, the hydrazinyl-indolyl or hydrazinyl-pyrrolo-pyridinyl coupling moieties may be attached (e.g., covalently attached) to a linker. As such, embodiments of the present disclosure include a hydrazinyl-indolyl or hydrazinyl-pyrrolo-pyridinyl coupling moiety attached to a drug (e.g., maytansine) through a linker. Various embodiments of the linker that may couple the hydrazinyl-indolyl or hydrazinyl-pyrrolo-pyridinyl coupling moiety to the drug (e.g., maytansine) are described in detail herein.

In certain embodiments, the antibody may be conjugated to a drug or active agent, where one or more amino acids of the antibody are modified before conjugation to the drug or active agent. Modification of one or more amino acids of the antibody may produce an antibody that contains one or more reactive groups suitable for conjugation to the drug or active agent. In some cases, the antibody may include one or more modified amino acid residues to provide one or more reactive groups suitable for conjugation to the moiety of interest (e.g., a moiety that includes a coupling moiety, such as a hydrazinyl-indolyl or a hydrazinyl-pyrrolo-pyridinyl coupling moiety as described above). For example, an amino acid of the antibody may be modified to include a reactive aldehyde group (e.g., a reactive aldehyde). A reactive aldehyde may be included in an “aldehyde tag” or “ald-tag”, which as used herein refers to an amino acid sequence derived from a sulfatase motif (e.g., L(C/S)TPSR) that has been converted by action of a formylglycine generating enzyme (FGE) to contain a 2-formylglycine residue (referred to herein as “fGly”). The fGly residue generated by an FGE may also be referred to as a “formylglycine”. Stated differently, the term “aldehyde tag” is used herein to refer to an amino acid sequence that includes a “converted” sulfatase motif (i.e., a sulfatase motif in which a cysteine or serine residue has been converted to fGly by action of an FGE, e.g., L(fGly)TPSR). A converted sulfatase motif may be produced from an amino acid sequence that includes an “unconverted” sulfatase motif (i.e., a sulfatase motif in which the cysteine or serine residue has not been converted to fGly by an FGE, but is capable of being converted, e.g., an unconverted sulfatase motif with the sequence: L(C/S)TPSR). By “conversion” as used in the context of action of a formylglycine generating enzyme (FGE) on a sulfatase motif refers to biochemical modification of a cysteine or serine residue in a sulfatase motif to a formylglycine (fGly) residue (e.g., Cys to fGly, or Ser to fGly). Additional aspects of aldehyde tags and uses thereof in site-specific protein modification are described in U.S. Pat. Nos. 7,985,783 and 8,729,232, the disclosures of each of which are incorporated herein by reference.

In some cases, to produce the conjugate, the antibody containing the fGly residue may be conjugated to the moiety of interest (e.g., drug or active agent) by reaction of the fGly with a compound (e.g., a compound containing a hydrazinyl-indolyl or a hydrazinyl-pyrrolo-pyridinyl coupling moiety, as described above). For example, an fGly-containing antibody may be contacted with a reactive partner-containing drug under conditions suitable to provide for conjugation of the drug to the antibody. In some instances, the reactive partner-containing drug may include a hydrazinyl-indolyl or a hydrazinyl-pyrrolo-pyridinyl coupling moiety as described above. For example, a maytansine may be modified to include a hydrazinyl-indolyl or a hydrazinyl-pyrrolo-pyridinyl coupling moiety. In some cases, the maytansine is attached to a hydrazinyl-indolyl or a hydrazinyl-pyrrolo-pyridinyl, such as covalently attached to a a hydrazinyl-indolyl or a hydrazinyl-pyrrolo-pyridinyl through a linker, as described in detail herein.

In certain embodiments, the method of the present disclosure involves the use of a ADC of Formula (I) which includes an antibody having at least one amino acid residue that has been attached to a moiety of interest (e.g., drug or active agent). In order to make the conjugate, an amino acid residue of the antibody may be modified and then coupled to a drug (e.g., maytansine) containing a hydrazinyl-indolyl or a hydrazinyl-pyrrolo-pyridinyl coupling moiety as described above. In certain embodiments, an amino acid residue of the antibody (e.g., anti-TACSTD2 antibody, a Muc-1 antibody, a Nectin-4 antibody, or a NaPi2B antibody) is a cysteine or serine residue that is modified to an fGly residue, as described above. In certain embodiments, the modified amino acid residue (e.g., fGly residue) is conjugated to a drug containing a hydrazinyl-indolyl or a hydrazinyl-pyrrolo-pyridinyl coupling moiety as described above to provide a conjugate of the present disclosure where the drug is conjugated to the antibody through the hydrazinyl-indolyl or hydrazinyl-pyrrolo-pyridinyl coupling moiety. As used herein, the term fGly′ refers to the amino acid residue of the antibody (e.g., anti-TACSTD2 antibody, a Muc-1 antibody, a Nectin-4 antibody, or a NaPi2B antibody) that is coupled to the moiety of interest (e.g., a drug, such as a maytansine).

In certain embodiments, the method as disclosed herein uses a conjugate that includes an antibody having at least one amino acid residue attached to a linker as described herein, which in turn is attached to a drug or active agent. For instance, the conjugate may include an antibody, having at least one amino acid residue (fGly′) that is conjugated to a drug (e.g., maytansine).

Aspects of the present disclosure include a conjugate of the formula (I):

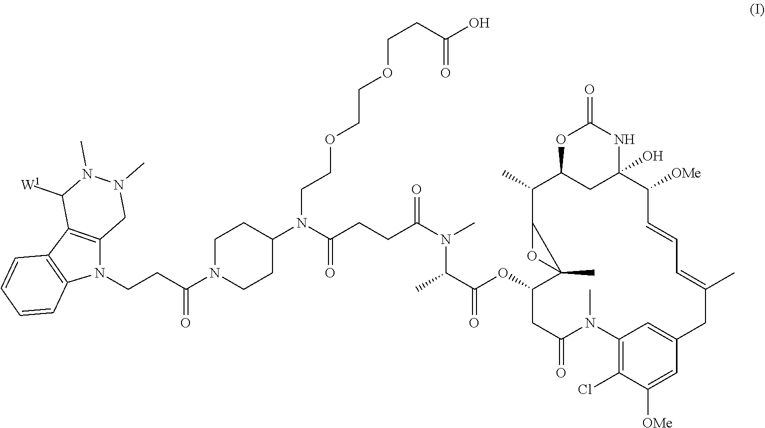

wherein W¹ is an antibody binding to an antigen, and wherein the administering reduces the toxicity in the subject associated with target-mediated cross-reactivity of the ADC.

In some instances, the antibody is an anti-nectin-4 antibody. In some instances, the antibody is an anti-Tumor Associated Calcium Signal Transducer 2 (TACSTD2) antibody. In some instances, the antibody is an anti-Muc1 antibody. In some instances, the antibody is an anti-NaPi2b antibody.

Additional disclosure related to antibodies and antibody-drug conjugates that find use in the present invention is found in U.S. Application Publication No. 2014/0141025, filed Mar. 11, 2013, U.S. Application Publication No. 2015/0157736, filed Nov. 26, 2014, U.S. application Ser. No. 17/389,723, filed Jul. 30, 2021, U.S. Application No. 63/236,988, filed Aug. 25, 2021, and U.S. Application No. 63/227,666, filed Jul. 30, 2021, the disclosures of each of which are incorporated herein by reference.

### Antibodies

As noted above, the methods as disclosed herein, includes administration of an antibody-drug-conjugate of Formula (I), wherein the antibody is targeted to a target antigen. As used herein, the antibody can be targeted to an antigen expressed on any vital organ of the subject. In certain aspects, the antibody can be targeted to target cells expressing the same antigen on the surface of a variety of target organs. For example, the antibody can bind to a target antigen expressed on any of or a combination of brain, heart, liver, kidney, pancreas, lungs, stomach, ovaries, breast, thyroid, skin of a subject.

### Anti-Tacstd2 Antibodies

As noted above, according to the methods of this invention, a subject can be administered conjugate that comprise, as substituent W¹ an antibody. In certain embodiments, the antibody can be an anti-TACSTD2 antibody, where the amino acid sequence of the anti-TACSTD2 antibody has been modified to include a 2-formylglycine (fGly) residue. As used herein, amino acids may be referred to by their standard name, their standard three letter abbreviation and/or their standard one letter abbreviation, such as: Alanine or Ala or A; Cysteine or Cys or C; Aspartic acid or Asp or D; Glutamic acid or Glu or E; Phenylalanine or Phe or F; Glycine or Gly or G; Histidine or His or H; Isoleucine or Ile or I; Lysine or Lys or K; Leucine or Leu or L; Methionine or Met or M; Asparagine or Asn or N; Proline or Pro or P; Glutamine or Gln or Q; Arginine or Arg or R; Serine or Ser or S; Threonine or Thr or T; Valine or Val or V; Tryptophan or Trp or W; and Tyrosine or Tyr or Y.

In some cases, a suitable anti-TACSTD2 antibody specifically binds a TACSTD2 polypeptide, where the epitope comprises amino acid residues within a TACSTD2 antigen. The amino acid sequence of a human TACSTD2 polypeptide (UniProtKB—P09758) is depicted in Table 3 below.

TABLE 3
Human TACSTD2 Amino Acid Sequence (UniProtKB-P11049)

| | |
|---|---|
| Human TACSTD2 | MARGPGLAPPPLRLPLLLLVLAAVTGHTAAQDNCTCPTNK |
| Amino Acid Sequence | MTVCSPDGPGGRCQCRALGSGMAVDCSTLTSKCLLLKARM |
| (SEQ ID NO: 12) | SAPKNARTLVRPSEHALVDNDGLYDPDCDPEGRFKARQCN |
| | QTSVCWCVNSVGVRRTDKGDLSLRCDELVRTHHILIDLRHR |
| | PTAGAFNHSDLDAELRRLFRERYRLHPKFVAAVHYEQPTIQI |
| | ELRQNTSQKAAGDVDIGDAAYYFERDIKGESLFQGRGGLDL |
| | RVRGEPLQVERTLIYYLDEIPPKFSMKRLTAGLIAVIVVVVV |
| | ALVAGMAVLVITNRRKSGKYKKVEIKELGELRKEPSL |

A TACSTD2 epitope can be formed by a polypeptide having at least about 75%, at least about 80%, at least about 85%, at least about 90%, at least about 95%, at least about 98%, at least about 99%, or 100%, amino acid sequence identity to a contiguous stretch of about four to about twenty amino acids of the human TACSTD2 amino acid sequence depicted in Table 3. A TACSTD2 epitope can also be a conformational epitope where the anti-TACSTD2 antibody binds to specific amino acids that are proximal to each other in a three-dimensional structure of TACSTD2; however are not contiguous in the sequence as depicted in SEQ ID NO: 12.

In some cases, a suitable anti-TACSTD2 antibody exhibits high affinity binding to TACSTD2. For example, in some cases, a suitable anti-TACSTD2 antibody binds to TACSTD2 with an affinity of at least about 10⁻⁷ M, at least about 10⁻⁸M, at least about 10⁻⁹ M, at least about 10⁻¹⁰ M, at least about 10⁻¹¹M, or at least about 10⁻¹² M, or greater than 10⁻¹² M. In some cases, a suitable anti-TACSTD2 antibody binds to an epitope present on TACSTD2 with an affinity of from about 10⁻⁷ M to about 10⁻⁸M, from about 10⁻⁸M to about 10⁻⁹ M, from about 10⁻⁹ M to about 10⁻¹⁰ M, from about 10⁻¹⁰ M to about 10⁻¹¹M, or from about 10⁻¹¹M to about 10⁻¹² M, or greater than 10⁻¹² M.

In some cases, a suitable anti-TACSTD2 antibody competes for binding to an epitope within TACSTD2 with a second anti-TACSTD2 antibody and/or binds to the same epitope within TACSTD2, as a second anti-TACSTD2 antibody. In some cases, an anti-TACSTD2 antibody that competes for binding to an epitope within TACSTD2 with a second anti-TACSTD2 antibody also binds to the same epitope as the second anti-TACSTD2 antibody. In some cases, an anti-TACSTD2 antibody that competes for binding to an epitope within TACSTD2 with a second anti-TACSTD2 antibody binds to an epitope that is overlapping with the epitope bound by the second anti-TACSTD2 antibody. In some cases, the anti-TACSTD2 antibody is humanized.

According to some embodiments, a conjugate of the present disclosure comprises an anti-TACSTD2 antibody that specifically binds to TACSTD2 and competes for binding to TACSTD2 with an anti-TACSTD2 antibody comprising:
a variable heavy chain (V_{H}) polypeptide comprising:
   a V_{H} CDR1 comprising the amino acid sequence NYNMN (SEQ ID NO: 1),
   a V_{H} CDR2 comprising the amino acid sequence WINTYTGEPTYTDDFKG (SEQ ID NO: 2), and
   a V_{H} CDR3 comprising the amino acid sequence GGFGSSYWYFDV (SEQ ID NO: 3); and
a variable light chain (V_{L}) polypeptide comprising:
   a V_{L} CDR1 comprising the amino acid sequence KASQDVSIAVA (SEQ ID NO: 4),
   a V_{L} CDR2 comprising the amino acid sequence SASYRYT (SEQ ID NO: 5), and
   a V_{L} CDR3 comprising the amino acid sequence QQHYITPLT (SEQ ID NO: 6).

In certain embodiments, a conjugate of the present disclosure comprises an anti-TACSTD2 antibody that comprises:
a variable heavy chain (V_{H}) polypeptide comprising:
   a V_{H} CDR1 comprising the amino acid sequence NYNMN (SEQ ID NO: 1),
   a V_{H} CDR2 comprising the amino acid sequence WINTYTGEPTYTDDFKG (SEQ ID NO: 2), and
   a V_{H} CDR3 comprising the amino acid sequence GGFGSSYWYFDV (SEQ ID NO: 3); and
a variable light chain (V_{L}) polypeptide comprising:
   a V_{L} CDR1 comprising the amino acid sequence KASQDVSIAVA (SEQ ID NO: 4),
   a V_{L} CDR2 comprising the amino acid sequence SASYRYT (SEQ ID NO: 5), and
   a V_{L} CDR3 comprising the amino acid sequence QQHYITPLT (SEQ ID NO: 6).

According to some embodiments, a conjugate of the present disclosure comprises an anti-TACSTD2 antibody comprising:
a variable heavy chain (V_{H}) polypeptide comprising an amino acid sequence having 70% or greater, 75% or greater, 80% or greater, 85% or greater, 90% or greater, 95% or greater, 99% or greater, or 100% identity to the amino acid sequence set forth in SEQ ID NO: 7; and
a variable light chain (V_{L}) polypeptide comprising an amino acid sequence having 70% or greater, 75% or greater, 80% or greater, 85% or greater, 90% or greater, 95% or greater, 99% or greater, or 100% identity to the amino acid sequence set forth in SEQ ID NO: 8.

Whether a first antibody “competes with” a second antibody for binding to TACSTD2 may be readily determined using competitive binding assays known in the art. Competing antibodies may be identified, for example, via an antibody competition assay. For example, a sample of a first antibody can be bound to a solid support. Then, a sample of a second antibody suspected of being able to compete with such first antibody is then added. One of the two antibodies is labelled. If the labeled antibody and the unlabeled antibody bind to separate and discrete sites on TACSTD2, the labeled antibody will bind to the same level whether or not the suspected competing antibody is present. However, if the sites of interaction are identical or overlapping, the unlabeled antibody will compete, and the amount of labeled antibody bound to TACSTD2 will be lowered. If the unlabeled antibody is present in excess, very little, if any, labeled antibody will bind.

For purposes of the present disclosure, competing antibodies are those that decrease the binding of an antibody to TACSTD2 by about 50% or more, about 60% or more, about 70% or more, about 80% or more, about 85% or more, about 90% or more, about 95% or more, or about 99% or more. Details of procedures for carrying out such competition assays are well known in the art and can be found, for example, in Harlow and Lane, Antibodies, A Laboratory Manual, Cold Spring Harbor Laboratory Press, Cold Spring Harbor, New York, 1988, 567-569, 1988, ISBN 0-87969-314-2. Such assays can be made quantitative by using purified antibodies. A standard curve may be established by titrating one antibody against itself, i.e., the same antibody is used for both the label and the competitor. The capacity of an unlabeled competing antibody to inhibit the binding of the labeled antibody to the plate may be titrated. The results may be plotted, and the concentrations necessary to achieve the desired degree of binding inhibition may be compared.

According to some embodiments, a conjugate of the present disclosure comprises an anti-TACSTD2 antibody comprising a heavy chain polypeptide comprising an amino acid sequence having 70% or greater, 75% or greater, 80% or greater, 85% or greater, 90% or greater, 95% or greater, 99% or greater, or 100% identity to the heavy chain polypeptide provided in Table 4. In certain embodiments, such an anti-TACSTD2 antibody comprises the V_{H} CDR1, V_{H}CDR2, and V_{H} CDR3 provided in Table 4.

According to some embodiments, a conjugate of the present disclosure comprises an anti-TACSTD2 antibody comprising a light chain polypeptide comprising an amino acid sequence having 70% or greater, 75% or greater, 80% or greater, 85% or greater, 90% or greater, 95% or greater, 99% or greater, or 100% identity to the light chain polypeptide provided in Table 4. In certain embodiments, such an anti-TACSTD2 antibody comprises the V_{L} CDR1, V_{L} CDR2, and V_{L} CDR3 provided in Table 4.

According to some embodiments, a conjugate of the present disclosure comprises an anti-TACSTD2 antibody comprising a heavy chain polypeptide comprising an amino acid sequence having 70% or greater, 75% or greater, 80% or greater, 85% or greater, 90% or greater, 95% or greater, 99% or greater, or 100% identity to the heavy chain polypeptide provided in Table 4; and a light chain polypeptide comprising an amino acid sequence having 70% or greater, 75% or greater, 80% or greater, 85% or greater, 90% or greater, 95% or greater, 99% or greater, or 100% identity to the light chain polypeptide provided in Table 4. In certain embodiments, such an anti-TACSTD2 antibody comprises the V_{H} CDR1, V_{H} CDR2, V_{H} CDR3, V_{L} CDR1, V_{L} CDR2, and V_{L} CDR3 provided in Table 4.

The amino acid sequences of the heavy chain polypeptide, V_{H} polypeptide, V_{H}CDRs, light chain polypeptide, V_{L} polypeptide and V_{L} CDRs of an example anti-TACSTD2 of the present disclosure are provided in Table 4 below (with CDRs according to Kabat in bold and variable regions underlined).

TABLE 4
Example Anti-TACSTD2 Antibody Amino Acid Sequences

| | |
|---|---|
| Nucleic acid sequence encoding | CAGGTCCAACTGCAGCAATCTGGGTCTGAGTTG |
| the heavy chain variable region | AAGAAGCCTGGGGCCTCAGTGAAGGTTTCCTGC |
| (SEQ ID NO: 18), CDR encoding | AAGGCTTCTGGATACACCTTCACAAACTATGGA |
| portions are underlined | ATGAACTGGGTGAAGCAGGCCCCTGGACAAGGG |
| | CTTAAATGGATGGGCTGGATAAACACCTACACT |
| | GGAGAGCCAACATATACTGATGACTTCAAGGGA |
| | CGGTTTGCCTTCTCCTTGGACACCTCTGTCAGCA |
| | CGGCATATCTCCAGATCAGCAGCCTAAAGGCTG |
| | ACGACACTGCCGTGTATTTCTGTGCAAGAGGGG |
| | GGTTCGGTAGTAGCTACTGGTACTTCGATGTCTG |
| | GGGCCAAGGGTCCCTGGTCACCGTCTCCTCA |
| Nucleic acid sequence encoding | GCCTCCACCAAGGGCCCATCGGTCTTCCCCCTGG |
| the heavy chain constant region | CACCCTCCTCCAAGAGCACCTCTGGGGGCACAG |
| (SEQ ID NO: 19), aldehyde tag | CGGCCCTGGGCTGCCTGGTCAAGGACTACTTCC |
| insertion is underlined. | CCGAACCGGTGACGGTGTCGTGGAACTCAGGCG |
| | CCCTGACCAGCGGCGTGCACACCTTCCCGGCTG |
| | TCCTACAGTCCTCAGGACTCTACTCCCTCAGCAG |
| | CGTGGTGACCGTGCCCTCCAGCAGCTTGGGCAC |
| | CCAGACCTACATCTGCAACGTGAATCACAAGCC |
| | CAGCAACACCAAGGTGGACAAGAAAGTT |
| | GAGCCCAAATCTTGTGACAAAACTCACACATGC |
| | CCACCGTGCCCA |
| | GCACCTGAACTCCTGGGGGGACCGTCAGTCTTC |
| | CTCTTCCCCCCAAAACCCAAGGACACCCTCATG |
| | ATCTCCCGGACCCCTGAGGTCACATGCGTGGTG |
| | GTGGACGTGAGCCACGAAGACCCTGAGGTCAAG |
| | TTCAACTGGTACGTGGACGGCGTGGAGGTGCAT |
| | AATGCCAAGACAAAGCCGCGGGAGGAGCAGTA |
| | CAACAGCACGTACCGTGTGGTCAGCGTCCTCAC |
| | CGTCCTGCACCAGGACTGGCTGAATGGCAAGGA |
| | GTACAAGTGCAAGGTCTCCAACAAAGCCCTCCC |
| | AGCCCCCATCGAGAAAACCATCTCCAAAGCCAA |
| | A |
| | GGGCAGCCCCGAGAACCACAGGTGTACACCCTG |
| | CCCCCATCCCGGGAAGAGATGACCAAGAACCAG |
| | GTCAGCCTGACCTGCCTGGTCAAAGGCTTCTATC |
| | CCAGCGACATCGCCGTGGAGTGGGAGAGCAATG |
| | GGCAGCCGGAGAACAACTACAAGACCACGCCTC |
| | CCGTGCTGGACTCCGACGGCTCCTTCTTCCTCTA |
| | TAGCAAGCTCACCGTGGACAAGAGCAGGTGGCA |
| | GCAGGGGAACGTCTTCTCATGCTCCGTGATGCA |
| | TGAGGCTCTGCACAACCACTACACGCAGAAGAG |
| | CCTCTCCCTGTCTCCGGGTTCA |
| | CTGTGTACCCCTTCTAGAGGATCCTGA |
| Heavy Chain protein sequence | QVQLQQSGSELKKPGASVKVSCKASGYTFTNYG |
| (SEQ ID NO: 9) | MNWVKQAPGQGLKWMGWINTYTGEPTYTDDF |
| V_{H} (SEQ ID NO: 7): | KGRFAFSLDTSVSTAYLQISSLKADDTAVYFCARG |
| (Underlined) | GFGSSYWYFDVWGQGSLVTVSSASTKGPSVFPLA |
| V_{H} CDR1 (SEQ ID NO: 1): | PSSKSTSGGTAALGCLVKDYFPEPVTVSWNSGALT |
| NYGMN | SGVHTFPAVLQSSGLYSLSSVVTVPSSSLGTQTYIC |
| V_{H} CDR2 (SEQ ID NO: 2): | NVNHKPSNTKVDKKVEPKSCDKTHTCPPCPAPELL |
| WINTYTGEPTYTDDFKG | GGPSVFLFPPKPKDTLMISRTPEVTCVVVDVSHED |
| V_{H} CDR3 (SEQ ID NO: 3) | PEVKFNWYVDGVEVHNAKTKPREEQYNSTYRVV |
| GGFGSSYWYFDV | SVLTVLHQDWLNGKEYKCKVSNKALPAPIEKTISK |
| | AKGQPREPQVYTLPPSREEMTKNQVSLTCLVKGF |
| | YPSDIAVEWESNGQPENNYKTTPPVLDSDGSFFLY |
| | SKLTVDKSRWQQGNVFSCSVMHEALHNHYTQKS |
| | LSLSPGSLCTPSRGS |
| Nucleic acid sequence encoding | GACATCCAGCTGACCCAGTCTCCATCCTCCCTGT |
| the Light Chain (SEQ ID NO: | CTGCATCTGTAGGAGACAGAGTCAGCATCACCT |
| 20), CDR encoding portions are | GCAAGGCCAGTCAGGATGTGAGTATTGCTGTAG |
| underlined | CCTGGTATCAGCAGAAACCAGGGAAAGCCCCTA |
| | AGCTCCTGATCTACTCGGCATCCTACCGGTACAC |
| | TGGAGTCCCTGATAGGTTCAGTGGCAGTGGATC |
| | TGGGACAGATTTCACTCTCACCATCAGCAGTCTG |
| | CAACCTGAAGATTTTGCAGTTTATTACTGTCAGC |
| | AACATTATATTACTCCGCTCACGTTCGGTGCTGG |
| | GACCAAGGTGGAGATCAAA |
| Nucleic acid sequence encoding | CGAACTGTGGCTGCACCATCTGTCTTCATCTTCC |
| the light chain constant region | CGCCATCTGATGAGCAGTTGAAATCTGGAACTG |
| (SEQ ID NO: 21). | CCTCTGTTGTGTGCCTGCTGAATAACTTCTATCC |
| | CAGAGAGGCCAAAGTACAGTGGAAGGTGGATA |
| | ACGCCCTCCAATCGGGTAACTCCCAGGAGAGTG |
| | TCACAGAGCAGGACAGCAAGGACAGCACCTAC |
| | AGCCTCAGCAGCACCCTGACGCTGAGCAAAGCA |
| | GACTACGAGAAACACAAAGTCTACGCCTGCGAA |
| | GTCACCCATCAGGGCCTGAGCTCGCCCGTCACA |
| | AAGAGCTTCAACAGGGGAGAGTGTTAG |
| Light Chain protein sequence | DIQLTQSPSSLSASVGDRVSITCKASQDVSIAVAW |
| (SEQ ID NO: 22) | YQQKPGKAPKLLIYSASYRYTGVPDRFSGSGSGTD |
| V_{L} (SEQ ID NO: 8): (Underlined) | FTLTISSLQPEDFAVYYCQQHYITPLTFGAGTKVEI |
| V_{L} CDR1 (SEQ ID NO: 4): | KRTVAAPSVFIFPPSDEQLKSGTASVVCLLNNFYPREAK |
| KASQDVSIAVA | VQWKVDNALQSGNSQESVTEQDSKDSTYSLSSTLTLSK |
| V_{L} CDR2 (SEQ ID NO: 5): | ADYEKHKVYACEVTHQGLSSPVTKSFNRGEC |
| SASYRYT | |
| V_{L} CDR3 (SEQ ID NO: 6): | |
| QQHYITPLT | |

According to some embodiments, a conjugate of the present disclosure comprises an anti-TACSTD2 antibody comprising a heavy chain polypeptide comprising an amino acid sequence having 70% or greater, 75% or greater, 80% or greater, 85% or greater, 90% or greater, 95% or greater, 99% or greater, or 100% identity to the heavy chain polypeptide provided in Table 4 (SEQ ID NO: 9), where the antibody comprises an L234A substitution, an L235A substitution, or both (e.g., an L234A substitution and an L235A substitution), where positions 234 and 235 are according to the EU numbering system. Edelman et al. (1969) Proc. Natl. Acad. 63:78-85. Residues L234 and L235 according to the EU numbering system are in bold and italicized in Table 4. These leucine residues are at positions 238 and 239 of SEQ ID NO: 9 provided in Table 4. In certain embodiments, such an anti-TACSTD2 antibody competes for binding to TACSTD2 with an antibody comprising the V_{H} CDR1, V_{H} CDR2, V_{H} CDR3, V_{L} CDR1, V_{L} CDR2, and V_{L} CDR3 set forth in Table 4. In certain embodiments, such an anti-TACSTD2 antibody comprises the V_{H} CDR1, V_{H} CDR2, V_{H} CDR3, V_{L} CDR1, V_{L} CDR2, and VL CDR3 set forth in Table 4.

In some embodiments, the anti-TACSTD2 antibody is an IgG1 antibody. For example, in certain aspects, the the anti-TACSTD2 antibody is an IgG1 kappa antibody.

In certain aspects, the anti-TACSTD2 antibody is a fGly′-containing antibody based on an antibody shown in Table 4. For example, in some embodiments, the antibody is a derivative of the antibody shown in Table 4, where the difference between the antibody and the derivative is the presence of one or more fGly′ residues (and optionally, the associated FGE recognition sequence amino acids) in the derivative. In the amino acid sequences in Table 4, variable regions are underlined and CDRs are shown in bold. In this example, the italicized residues at the C-terminus of the heavy chain replace a lysine residue at the C-terminus of a standard IgG1 heavy chain. The underlined residues (LCTPSR) among the italicized residues constitute the aldehyde tag, where the C is converted to an fGly residue by FGE upon expression of the heavy chain. The non-underlined residues among the italicized residues are additional residues that are different from a standard IgG1 heavy chain sequence.

In some embodiments, the anti-TACSTD2 antibody comprises one, two, three, four, five, or all six complementarity determining regions (CDRs) of the anti-TACSTD2 antibody sacituzumab.

In certain aspects, the anti-TACSTD2 antibody is a fGly′-containing antibody based on an antibody shown in Table 4. For example, in some embodiments, the antibody is a derivative of the antibody shown in Table 4, where the difference between the antibody and the derivative is the presence of one or more fGly′ residues (and optionally, the associated FGE recognition sequence amino acids) in the derivative. Provided in Table 4 are exemplary nucleic acid and amino acid sequences for sacituzumab-based antibody according to one embodiment of the disclosure. In the amino acid sequences in Table 4, variable regions are underlined and CDRs are shown in bold. In this example of sacituzumab-based antibody, the italicized residues at the C-terminus of the heavy chain replace a lysine residue at the C-terminus of a standard IgG1 heavy chain. The underlined residues (LCTPSR) among the italicized residues constitute the aldehyde tag, where the C is converted to an fGly residue by FGE upon expression of the heavy chain. The non-underlined residues among the italicized residues are additional residues that are different from a standard IgG1 heavy chain sequence.

An anti-TACSTD2 antibody suitable for use in a subject conjugate will in some cases inhibit the proliferation of human tumor cells that express on their surface (e.g., overexpress) TACSTD2, where the inhibition occurs in vitro, in vivo, or both in vitro and in vivo. For example, in some cases, an anti-TACSTD2 antibody suitable for use in a subject conjugate inhibits proliferation of human tumor cells that express on their surface (e.g., overexpress) TACSTD2 by at least about 15%, at least about 20%, at least about 25%, at least about 30%, at least about 40%, at least about 50%, at least about 60%, at least about 70%, at least about 80%, or more than 80%, e.g., by at least about 85%, at least about 90%, at least about 95%, at least about 98%, at least about 99%, or 100%.

### Modified Constant Region Sequences

As noted above, the amino acid sequence of an anti-TACSTD2 antibody can be modified to include a sulfatase motif that contains a serine or cysteine residue that is capable of being converted (oxidized) to a 2-formylglycine (fGly) residue by action of a formylglycine generating enzyme (FGE) either in vivo (e.g., at the time of translation of an aldehyde tag-containing protein in a cell) or in vitro (e.g., by contacting an aldehyde tag-containing protein with an FGE in a cell-free system). Such sulfatase motifs may also be referred to herein as an FGE-modification site.

### Sulfatase Motifs

A minimal sulfatase motif of an aldehyde tag is usually 5 or 6 amino acid residues in length, usually no more than 6 amino acid residues in length. Sulfatase motifs provided in an Ig polypeptide are at least 5 or 6 amino acid residues, and can be, for example, from 5 to 16, 6-16, 5-15, 6-15, 5-14, 6-14, 5-13, 6-13, 5-12, 6-12, 5-11, 6-11, 5-10, 6-10, 5-9, 6-9, 5-8, or 6-8 amino acid residues in length, so as to define a sulfatase motif of less than 16, 15, 14, 13, 12, 11, 10, 9, 8 or 7 amino acid residues in length.

In certain embodiments, polypeptides of interest include those where one or more amino acid residues, such as 2 or more, or 3 or more, or 4 or more, or 5 or more, or 6 or more, or 7 or more, or 8 or more, or 9 or more, or 10 or more, or 11 or more, or 12 or more, or 13 or more, or 14 or more, or 15 or more, or 16 or more, or 17 or more, or 18 or more, or 19 or more, or 20 or more amino acid residues have been inserted, deleted, substituted (replaced) relative to the native amino acid sequence to provide for a sequence of a sulfatase motif in the polypeptide. In certain embodiments, the polypeptide includes a modification (insertion, addition, deletion, and/or substitution/replacement) of less than 20, 19, 18, 17, 16, 15, 14, 13, 12, 11, 10, 9, 8, 7, 6, 5, 4, 3 or 2 amino acid residues of the amino acid sequence relative to the native amino acid sequence of the polypeptide. Where an amino acid sequence native to the polypeptide (e.g., anti-TACSTD2 antibody) contains one or more residues of the desired sulfatase motif, the total number of modifications of residues can be reduced, e.g., by site-specification modification (insertion, addition, deletion, substitution/replacement) of amino acid residues flanking the native amino acid residues to provide a sequence of the desired sulfatase motif. In certain embodiments, the extent of modification of the native amino acid sequence of the target anti-TACSTD2 polypeptide is minimized, so as to minimize the number of amino acid residues that are inserted, deleted, substituted (replaced), or added (e.g., to the N- or C-terminus). Minimizing the extent of amino acid sequence modification of the target anti-TACSTD2 polypeptide may minimize the impact such modifications may have upon anti-TACSTD2 function and/or structure.

It should be noted that while aldehyde tags of particular interest are those comprising at least a minimal sulfatase motif (also referred to a “consensus sulfatase motif”), it will be readily appreciated that longer aldehyde tags are both contemplated and encompassed by the present disclosure and can find use in the compositions and methods of the present disclosure. Aldehyde tags can thus comprise a minimal sulfatase motif of 5 or 6 residues, or can be longer and comprise a minimal sulfatase motif which can be flanked at the N- and/or C-terminal sides of the motif by additional amino acid residues. Aldehyde tags of, for example, 5 or 6 amino acid residues are contemplated, as well as longer amino acid sequences of more than 5, 6, 7, 8, 9, 10, 11, 12, 13, 14, 15, 16, 17, 18, 19, 20 or more amino acid residues.

An aldehyde tag can be present at or near the C-terminus of an Ig heavy chain; e.g., an aldehyde tag can be present within 1, 2, 3, 4, 5, 6, 7, 8, 9, or 10 amino acids of the C-terminus of a native, wild-type Ig heavy chain. An aldehyde tag can be present within a CH1 domain of an Ig heavy chain. An aldehyde tag can be present within a CH2 domain of an Ig heavy chain. An aldehyde tag can be present within a CH3 domain of an Ig heavy chain. An aldehyde tag can be present in an Ig light chain constant region, e.g., in a kappa light chain constant region or a lambda light chain constant region.

In certain embodiments, the sulfatase motif used may be described by the formula:

X¹Z¹⁰X²Z²⁰X³Z³⁰ (I′)

where
Z¹⁰ is cysteine or serine (which can also be represented by (C/S));
Z²⁰ is either a proline or alanine residue (which can also be represented by (P/A));
Z³⁰ is a basic amino acid (e.g., arginine I, and may be lysine (K) or histidine (H), e.g., lysine), or an aliphatic amino acid (alanine (A), glycine (G), leucine (L), valine (V), isoleucine (I), or proline (P), e.g., A, G, L, V, or I;
X¹ is present or absent and, when present, can be any amino acid, e.g., an aliphatic amino acid, a sulfur-containing amino acid, or a polar, uncharged amino acid, (i.e., other than an aromatic amino acid or a charged amino acid), e.g., L, M, V, S or T, e.g., L, M, S or V, with the proviso that when the sulfatase motif is at the N-terminus of the target polypeptide, X¹ is present; and
X² and X³ independently can be any amino acid, though usually an aliphatic amino acid, a polar, uncharged amino acid, or a sulfur containing amino acid (i.e., other than an aromatic amino acid or a charged amino acid), e.g., S, T, A, V, G or C, e.g., S, T, A, V or G.

The amino acid sequence of an anti-TACSTD2 heavy and/or light chain can be modified to provide a sequence of at least 5 amino acids of the formula X¹Z¹⁰X²Z²⁰X³Z³⁰, where
Z¹⁰ is cysteine or serine;
Z²⁰ is a proline or alanine residue;
Z³⁰ is an aliphatic amino acid or a basic amino acid;
X¹ is present or absent and, when present, is any amino acid, with the proviso that when the heterologous sulfatase motif is at an N-terminus of the polypeptide, X¹ is present;
X² and X³ are each independently any amino acid,
where the sequence is within or adjacent a solvent-accessible loop region of the Ig constant region, and wherein the sequence is not at the C-terminus of the Ig heavy chain.

The sulfatase motif is generally selected so as to be capable of conversion by a selected FGE, e.g., an FGE present in a host cell in which the aldehyde tagged polypeptide is expressed or an FGE which is to be contacted with the aldehyde tagged polypeptide in a cell-free in vitro method.

For example, where the FGE is a eukaryotic FGE (e.g., a mammalian FGE, including a human FGE), the sulfatase motif can be of the formula:

X¹CX²PX³Z³⁰ (I″)

where
X¹ may be present or absent and, when present, can be any amino acid, e.g., an aliphatic amino acid, a sulfur-containing amino acid, or a polar, uncharged amino acid, (i.e., other than an aromatic amino acid or a charged amino acid), e.g., L, M, S or V, with the proviso that when the sulfatase motif is at the N-terminus of the target polypeptide, X¹ is present;
X² and X³ independently can be any amino acid, e.g., an aliphatic amino acid, a sulfur-containing amino acid, or a polar, uncharged amino acid, (i.e., other than an aromatic amino acid or a charged amino acid), e.g., S, T, A, V, G, or C, e.g., S, T, A, V or G; and
Z³⁰ is a basic amino acid (e.g., arginiI(R), and may be lysine (K) or histidine (H), e.g., lysine), or an aliphatic amino acid (alanine (A), glycine (G), leucine (L), valine (V), isoleucine (I), or proline (P), e.g., A, G, L, V, or I.

Specific examples of sulfatase motifs include LCTPSR (SEQ ID NO: 13), MCTPSR (SEQ ID NO: 14), VCTPSR (SEQ ID NO: 15), LCSPSR (SEQ ID NO: 16), LCAPSR (SEQ ID NO: 17), LCVPSR (SEQ ID NO: 10), LCGPSR (SEQ ID NO: 11), ICTPAR (SEQ ID NO: 23), LCTPSK (SEQ ID NO: 24), MCTPSK (SEQ ID NO: 25), VCTPSK (SEQ ID NO: 26), LCSPSK (SEQ ID NO: 27), LCAPSK (SEQ ID NO: 28), LCVPSK (SEQ ID NO: 29), LCGPSK (SEQ ID NO: 30), LCTPSA (SEQ ID NO: 31), ICTPAA (SEQ ID NO: 32), MCTPSA (SEQ ID NO: 33), VCTPSA (SEQ ID NO: 34), LCSPSA (SEQ ID NO: 35), LCAPSA (SEQ ID NO: 36), LCVPSA (SEQ ID NO: 37), and LCGPSA (SEQ ID NO: 38).

fGly-Containing Sequences

Upon action of FGE on the anti-TACSTD2 heavy and/or light chain, the serine or the cysteine in the sulfatase motif is modified to fGly. Thus, the fGly-containing sulfatase motif can be of the formula:

X¹(fGly)X²Z²⁰X³Z³⁰ (I′″)

where
fGly is the formylglycine residue;
Z²⁰ is either a proline or alanine residue (which can also be represented by (P/A));
Z⁰ is a basic amino acid (e.g., argIne (R), and may be lysine (K) or histidine (H), usually lysine), or an aliphatic amino acid (alanine (A), glycine (G), leucine (L), valine (V), isoleucine (I), or proline (P), e.g., A, G, L, V, or I;
X¹ may be present or absent and, when present, can be any amino acid, e.g., an aliphatic amino acid, a sulfur-containing amino acid, or a polar, uncharged amino acid, (i.e., other than an aromatic amino acid or a charged amino acid), e.g., L, M, V, S or T, e.g., L, M or V, with the proviso that when the sulfatase motif is at the N-terminus of the target polypeptide, X¹ is present; and
X² and X³ independently can be any amino acid, e.g., an aliphatic amino acid, a sulfur-containing amino acid, or a polar, uncharged amino acid, (i.e., other than an aromatic amino acid or a charged amino acid), e.g., S, T, A, V, G or C, e.g., S, T, A, V or G.

As described above, to produce the conjugate, the polypeptide containing the fGly residue may be conjugated to a drug or active agent (e.g., a maytansinoid) by reaction of the fGly with a reactive moiety (e.g., hydrazinyl-indolyl or a hydrazinyl-pyrrolo-pyridinyl coupling moiety, as described above) of a linker attached to the drug or active agent to produce an fGly′-containing sulfatase motif. As used herein, the term fGly′ refers to the amino acid residue of the sulfatase motif that is coupled to the drug or active agent (such as a maytansinoid) through a linker as described herein. Thus, the fGly′-containing sulfatase motif can be of the formula:

X¹(fGly′)X²Z²⁰X³Z³⁰ (II)

where
fGly′ is the amino acid residue coupled to the drug or active agent through a linker as described herein;
Z²⁰ is either a proline or alanine residue (which can also be represented by (P/A));
Z³⁰ is a basic amino acid (e.g., Iinine (R), and may be lysine (K) or histidine (H), usually lysine), or an aliphatic amino acid (alanine (A), glycine (G), leucine (L), valine (V), isoleucine (I), or proline (P), e.g., A, G, L, V, or I;
X¹ may be present or absent and, when present, can be any amino acid, e.g., an aliphatic amino acid, a sulfur-containing amino acid, or a polar, uncharged amino acid, (i.e., other than an aromatic amino acid or a charged amino acid), e.g., L, M, V, S or T, e.g., L, M or V, with the proviso that when the sulfatase motif is at the N-terminus of the target polypeptide, X¹ is present; and
X² and X³ independently can be any amino acid, e.g., an aliphatic amino acid, a sulfur-containing amino acid, or a polar, uncharged amino acid, (i.e., other than an aromatic amino acid or a charged amino acid), e.g., S, T, A, V, G or C, e.g., S, T, A, V or G.

In certain embodiments, the sequence of formula (II) is positioned at a C-terminus of a heavy chain constant region of the anti-TACSTD2 antibody. In some instances, the heavy chain constant region comprises a sequence of the formula (II):

X¹(fGly′)X²Z²⁰X³Z³⁰ (II)

where
fGly′ is the amino acid residue coupled to the drug or active agent through a linker as described herein;
Z²⁰ is either a proline or alanine residue (which can also be represented by (P/A));
Z³⁰ is a basic amino acid (e.g., arginine (R), and may be lysine (K) or histidine (H), usually lysine), or an aliphatic amino acid (alanine (A), glycine (G), leucine (L), valine (V), isoleucine (I), or proline (P), e.g., A, G, L, V, or I;
X¹ may be present or absent and, when present, can be any amino acid, e.g., an aliphatic amino acid, a sulfur-containing amino acid, or a polar, uncharged amino acid, (i.e., other than an aromatic amino acid or a charged amino acid), e.g., L, M, V, S or T, e.g., L, M or V, with the proviso that when the sulfatase motif is at the N-terminus of the target polypeptide, X¹ is present;
X² and X³ independently can be any amino acid, e.g., an aliphatic amino acid, a sulfur-containing amino acid, or a polar, uncharged amino acid, (i.e., other than an aromatic amino acid or a charged amino acid), e.g., S, T, A, V, G or C, e.g., S, T, A, V or G; and
wherein the sequence is C-terminal to the amino acid sequence QKSLSLSPGK, and where the sequence may include 1, 2, 3, 4, 5, or from 5 to 10, amino acids that are not present in a native, wild-type heavy Ig chain constant region.

In certain embodiments, the heavy chain constant region comprises the sequence SLSLSPGSL(fGly′)TPSRGS (SEQ ID NO: 39) at the C-terminus of the Ig heavy chain, e.g., in place of a native SLSLSPGK (SEQ ID NO: 40) sequence.

In certain embodiments, the amino acid residue coupled to the drug or active agent (fGly′) is positioned in a light chain constant region of the anti-TACSTD2 antibody. In certain embodiments, the light chain constant region comprises a sequence of the formula (II):

X¹(fGly′)X²Z²⁰X³Z³⁰ (II)

where
fGly′ is the amino acid residue coupled to the drug or active agent through a linker as described herein;
Z²⁰ is either a proline or alanine residue (which can also be represented by (P/A));
Z³⁰ is a basic amino acid (e.g., arginine (R), and may be lysine (K) or histidine (H), usually lysine), or an aliphatic amino acid (alanine (A), glycine (G), leucine (L), valine (V), isoleucine (I), or proline (P), e.g., A, G, L, V, or I;
X¹ may be present or absent and, when present, can be any amino acid, e.g., an aliphatic amino acid, a sulfur-containing amino acid, or a polar, uncharged amino acid, (i.e., other than an aromatic amino acid or a charged amino acid), e.g., L, M, V, S or T, e.g., L, M or V, with the proviso that when the sulfatase motif is at the N-terminus of the target polypeptide, X¹ is present;
X² and X³ independently can be any amino acid, e.g., an aliphatic amino acid, a sulfur-containing amino acid, or a polar, uncharged amino acid, (i.e., other than an aromatic amino acid or a charged amino acid), e.g., S, T, A, V, G or C, e.g., S, T, A, V or G; and
wherein the sequence is C-terminal to the amino acid sequence KVDNAL (SEQ ID NO: 41) and/or is N-terminal to the amino acid sequence QSGNSQ (SEQ ID NO: 42).

In certain embodiments, the light chain constant region comprises the sequence KVDNAL(fGly′)TPSRQSGNSQ (SEQ ID NO: 43).

In certain embodiments, the amino acid residue coupled to the drug or active agent (fGly′) is positioned in a heavy chain CH1 region of the anti-TACSTD2 antibody. In certain embodiments, the heavy chain CH1 region comprises a sequence of the formula (II):

X¹(fGly′)X²Z²⁰X³Z³⁰ (II)

where
fGly′ is the amino acid residue coupled to the drug or active agent through a linker as described herein;
Z²⁰ is either a proline or alanine residue (which can also be represented by (P/A));
Z³⁰ is a basic amino acid (e.g., arginine (R), and may be lysine (K) or histidine (H), usually lysine), or an aliphatic amino acid (alanine (A), glycine (G), leucine (L), valine (V), isoleucine (I), or proline (P), e.g., A, G, L, V, or I;
X¹ may be present or absent and, when present, can be any amino acid, e.g., an aliphatic amino acid, a sulfur-containing amino acid, or a polar, uncharged amino acid, (i.e., other than an aromatic amino acid or a charged amino acid), e.g., L, M, V, S or T, e.g., L, M or V, with the proviso that when the sulfatase motif is at the N-terminus of the target polypeptide, X¹ is present;
X² and X³ independently can be any amino acid, e.g., an aliphatic amino acid, a sulfur-containing amino acid, or a polar, uncharged amino acid, (i.e., other than an aromatic amino acid or a charged amino acid), e.g., S, T, A, V, G or C, e.g., S, T, A, V or G; and
wherein the sequence is C-terminal to the amino acid sequence SWNSGA (SEQ ID NO: 44) and/or is N-terminal to the amino acid sequence GVHTFP (SEQ ID NO: 45).

In certain embodiments, the heavy chain CH1 region comprises the sequence SWNSGAL(fGly′)TPSRGVHTFP (SEQ ID NO: 46).

### Site of Modification

As noted above, the amino acid sequence of an anti-TACSTD2 antibody can be modified to include a sulfatase motif that contains a serine or cysteine residue that is capable of being converted (oxidized) to an fGly residue by action of an FGE either in vivo (e.g., at the time of translation of an aldehyde tag-containing protein in a cell) or in vitro (e.g., by contacting an aldehyde tag-containing protein with an FGE in a cell-free system). The anti-TACSTD2 polypeptides used to generate a conjugate of the present disclosure include at least an Ig constant region, e.g., an Ig heavy chain constant region (e.g., at least a CH1 domain; at least a CH1 and a CH2 domain; a CH1, a CH2, and a CH3 domain; or a CH1, a CH2, a CH3, and a CH4 domain), or an Ig light chain constant region. Such Ig polypeptides are referred to herein as “target Ig polypeptides” or “target anti-TACSTD2 antibodies” or “target anti-TACSTD2 Ig polypeptides.”

The site in an anti-TACSTD2 antibody into which a sulfatase motif is introduced can be any convenient site. As noted above, in some instances, the extent of modification of the native amino acid sequence of the target anti-TACSTD2 polypeptide is minimized, so as to minimize the number of amino acid residues that are inserted, deleted, substituted (replaced), and/or added (e.g., to the N- or C-terminus). Minimizing the extent of amino acid sequence modification of the target anti-TACSTD2 polypeptide may minimize the impact such modifications may have upon anti-TACSTD2 function and/or structure.

An anti-TACSTD2 antibody heavy chain constant region can include Ig constant regions of any heavy chain isotype, non-naturally occurring Ig heavy chain constant regions (including consensus Ig heavy chain constant regions). An Ig constant region amino acid sequence can be modified to include an aldehyde tag, where the aldehyde tag is present in or adjacent a solvent-accessible loop region of the Ig constant region. An Ig constant region amino acid sequence can be modified by insertion and/or substitution of 1, 2, 3, 4, 5, 6, 7, 8, 9, 10, 11, 12, 13, 14, 15, or 16 amino acids, or more than 16 amino acids, to provide an amino acid sequence of a sulfatase motif as described above.

In some cases, an aldehyde-tagged anti-TACSTD2 antibody comprises an aldehyde-tagged Ig heavy chain constant region (e.g., at least a CH1 domain; at least a CH1 and a CH2 domain; a CH1, a CH2, and a CH3 domain; or a CH1, a CH2, a CH3, and a CH4 domain). The aldehyde-tagged Ig heavy chain constant region can include heavy chain constant region sequences of an IgA, IgM, IgD, IgE, IgG1, IgG2, IgG3, or IgG4 isotype heavy chain or any allotypic variant of same, e.g., human heavy chain constant region sequences or mouse heavy chain constant region sequences, a hybrid heavy chain constant region, a synthetic heavy chain constant region, or a consensus heavy chain constant region sequence, etc., that includes at least one sulfatase motif that can be modified by an FGE to generate an fGly-modified Ig polypeptide. Allotypic variants of Ig heavy chains are known in the art. See, e.g., Jefferis and Lefranc (2009) MAbs 1:4.

In some cases, an aldehyde-tagged anti-TACSTD2 antibody comprises an aldehyde-tagged Ig light chain constant region. The aldehyde-tagged Ig light chain constant region can include constant region sequences of a kappa light chain, a lambda light chain, e.g., human kappa or lambda light chain constant regions, a hybrid light chain constant region, a synthetic light chain constant region, or a consensus light chain constant region sequence, etc., that includes at least one sulfatase motif that can be modified by an FGE to generate an fGly-modified anti-TACSTD2 antibody polypeptide. Exemplary constant regions include human gamma 1 and gamma 3 regions. With the exception of the sulfatase motif, a constant region may have a wild-type amino acid sequence, or it may have an amino acid sequence that is at least 70% identical (e.g., at least 80%, at least 90% or at least 95% identical) to a wild type amino acid sequence.

In some embodiments the sulfatase motif is at a position other than, or in addition to, the C-terminus of the Ig polypeptide heavy chain. As noted above, an isolated aldehyde-tagged anti-TACSTD2 polypeptide can comprise a heavy chain constant region amino acid sequence modified to include a sulfatase motif as described above, where the sulfatase motif is in or adjacent to a surface-accessible loop region of the anti-TACSTD2 polypeptide heavy chain constant region.

In some instances, a target anti-TACSTD2 immunoglobulin amino acid sequence is modified to include a sulfatase motif as described above, where the modification includes one or more amino acid residue insertions, deletions, and/or substitutions. In certain embodiments, the sulfatase motif is within, or adjacent to, a region of an IgG1 heavy chain constant region corresponding to one or more of: 1) amino acids 122-127; 2) amino acids 137-143; 3) amino acids 155-158; 4) amino acids 163-170; 5) amino acids 163-183; 6) amino acids 179-183; 7) amino acids 190-192; 8) amino acids 200-202; 9) amino acids 199-202; 10) amino acids 208-212; 11) amino acids 220-241; 12) amino acids 247-251; 13) amino acids 257-261; 14) amino acid 269-277; 15) amino acids 271-277; 16) amino acids 284-285; 17) amino acids 284-292; 18) amino acids 289-291; 19) amino acids 299-303; 20) amino acids 309-313; 21) amino acids 320-322; 22) amino acids 329-335; 23) amino acids 341-349; 24) amino acids 342-348; 25) amino acids 356-365; 26) amino acids 377-381; 27) amino acids 388-394; 28) amino acids 398-407; 29) amino acids 433-451; and 30) amino acids 446-451; wherein the amino acid numbering is based on the amino acid numbering of human IgG1.

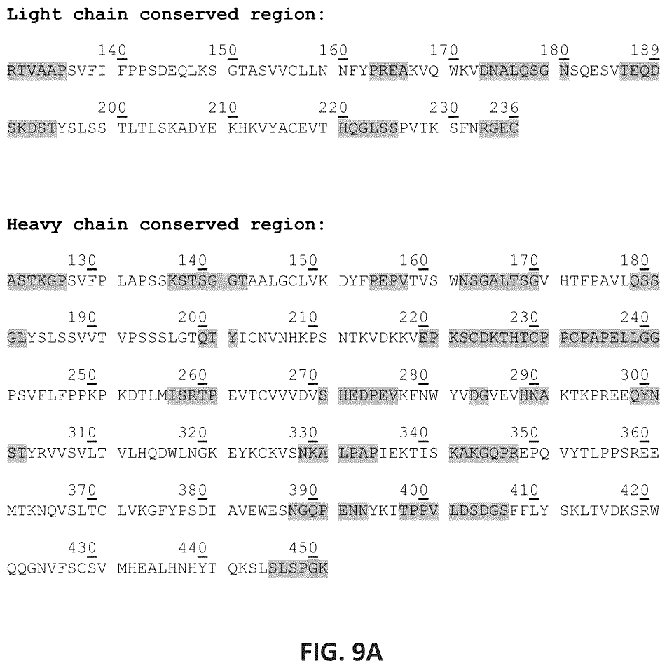

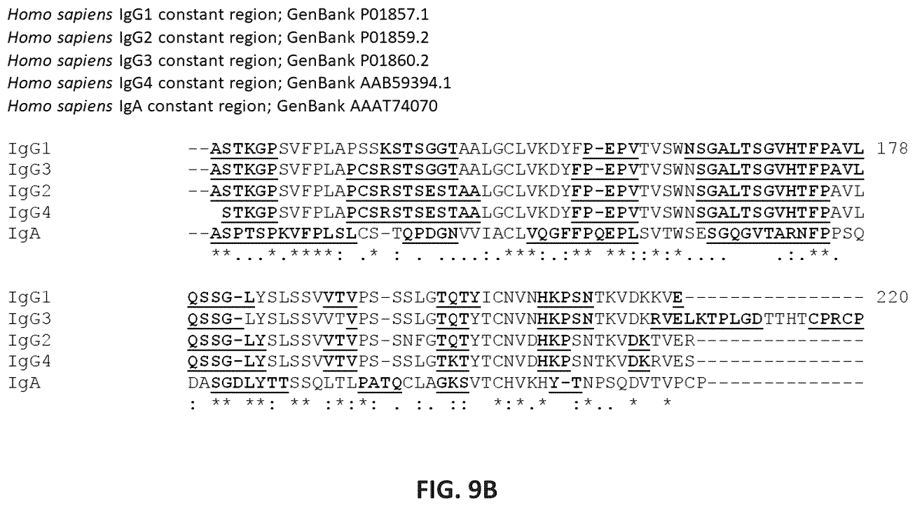

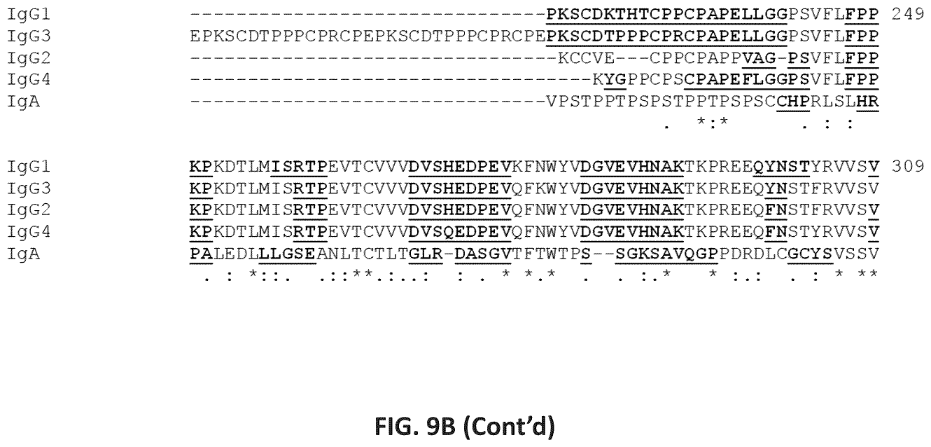

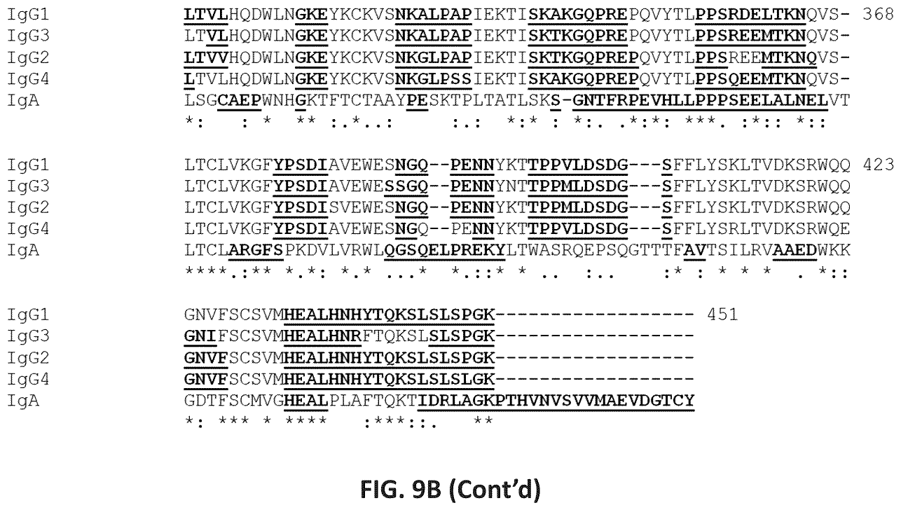

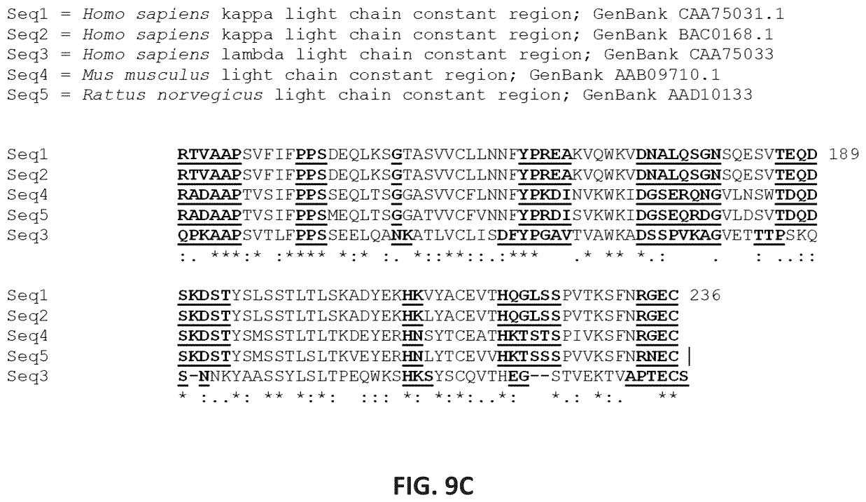

In some instances, a target anti-TACSTD2 immunoglobulin amino acid sequence is modified to include a sulfatase motif as described above, where the modification includes one or more amino acid residue insertions, deletions, and/or substitutions. In certain embodiments, the sulfatase motif is within, or adjacent to, a region of an IgG1 heavy chain constant region corresponding to one or more of: 1) amino acids 1-6; 2) amino acids 16-22; 3) amino acids 34-47; 4) amino acids 42-49; 5) amino acids 42-62; 6) amino acids 34-37; 7) amino acids 69-71; 8) amino acids 79-81; 9) amino acids 78-81; 10) amino acids 87-91; 11) amino acids 100-121; 12) amino acids 127-131; 13) amino acids 137-141; 14) amino acid 149-157; 15) amino acids 151-157; 16) amino acids 164-165; 17) amino acids 164-172; 18) amino acids 169-171; 19) amino acids 179-183; 20) amino acids 189-193; 21) amino acids 200-202; 22) amino acids 209-215; 23) amino acids 221-229; 24) amino acids 22-228; 25) amino acids 236-245; 26) amino acids 217-261; 27) amino acids 268-274; 28) amino acids 278-287; 29) amino acids 313-331; and 30) amino acids 324-331; wherein the amino acid numbering is based on the amino acid numbering of human IgG1 as set out in SEQ ID NO: 47 (human IgG1 constant region) as depicted in FIG. 9B.

Exemplary surface-accessible loop regions of an IgG1 heavy chain include: 1)

1)
(SEQ ID NO: 48)
ASTKGP;
2)
(SEQ ID NO: 49)
KSTSGGT;
3)
(SEQ ID NO: 50)
PEPV;
4)
(SEQ ID NO: 51)
NSGALTSG;
5)
(SEQ ID NO: 52)
NSGALTSGVHTFPAVLQSSGL;
6)
(SEQ ID NO: 53)
QSSGL;
7)
VTV;
8)
QTY;
9)
(SEQ ID NO: 54)
TQTY;
10)
(SEQ ID NO: 55)
HKPSN;
11)
(SEQ ID NO: 56)
EPKSCDKTHTCPPCPAPELLGG;
12)
(SEQ ID NO: 57)
FPPKP;
13)
(SEQ ID NO: 58)
ISRTP;
14)
(SEQ ID NO: 59)
DVSHEDPEV;
15)
(SEQ ID NO: 60)
SHEDPEV;
16)
DG;
17)
(SEQ ID NO: 61)
DGVEVHNAK;
18)
HNA;
19)
(SEQ ID NO: 62)
QYNST;
20)
(SEQ ID NO: 63)
VLTVL;
21)
GKE;
22)
(SEQ ID NO: 64)
NKALPAP;
23)
(SEQ ID NO: 65)
SKAKGQPRE;
24)
(SEQ ID NO: 66)
KAKGQPR;
25)
(SEQ ID NO: 67)
PPSRKELTKN;
26)
(SEQ ID NO: 68)
YPSDI;
27)
(SEQ ID NO: 69)
NGQPENN;
28)
(SEQ ID NO: 70)
TPPVLDSDGS;
29)
(SEQ ID NO: 71)
HEALHNHYTQKSLSLSPGK;
and
30)
(SEQ ID NO: 72)
SLSPGK.

In some instances, a target immunoglobulin amino acid sequence is modified to include a sulfatase motif as described above, where the modification includes one or more amino acid residue insertions, deletions, and/or substitutions. In certain embodiments, the sulfatase motif is within, or adjacent to, a region of an IgG2 heavy chain constant region corresponding to one or more of: 1) amino acids 1-6; 2) amino acids 13-24; 3) amino acids 33-37; 4) amino acids 43-54; 5) amino acids 58-63; 6) amino acids 69-71; 7) amino acids 78-80; 8) 87-89; 9) amino acids 95-96; 10) 114-118; 11) 122-126; 12) 134-136; 13) 144-152; 14) 159-167; 15) 175-176; 16) 184-188; 17) 195-197; 18) 204-210; 19) 216-224; 20) 231-233; 21) 237-241; 22) 252-256; 23) 263-269; 24) 273-282; 25) amino acids 299-302; where the amino acid numbering is based on the numbering of the amino acid sequence set forth in SEQ ID NO: 73 (human IgG2) as depicted in FIG. 9B.

1)
(SEQ ID NO: 74)
ASTKGP;
2)
(SEQ ID NO: 75)
PCSRSTSESTAA;
3)
(SEQ ID NO: 76)
FPEPV;
4)
(SEQ ID NO: 77)
SGALTSGVHTFP;
5)
(SEQ ID NO: 78)
QSSGLY;
6)
VTV;
7)
TQT;
8)
HKP;
9)
DK;
10)
(SEQ ID NO: 79)
VAGPS;
11)
(SEQ ID NO: 80)
FPPKP;
12)
RTP;
13)
(SEQ ID NO: 81)
DVSHEDPEV;
14)
(SEQ ID NO: 82)
DGVEVHNAK;
15)
FN;
16)
(SEQ ID NO: 83)
VLTVV;
17)
GKE;
18)
(SEQ ID NO: 84)
NKGLPAP;
19)
(SEQ ID NO: 85)
SKTKGQPRE;
20)
PPS;
21)
(SEQ ID NO: 86)
MTKNQ;
22)
(SEQ ID NO: 87)
YPSDI;
23)
(SEQ ID NO: 88)
NGQPENN ;
24)
(SEQ ID NO: 89)
TPPMLDSDGS;
25)
(SEQ ID NO: 90)
GNVF;
and
26)
(SEQ ID NO: 91)
HEALHNHYTQKSLSLSPGK.

In some instances, a target immunoglobulin amino acid sequence is modified to include a sulfatase motif as described above, where the modification includes one or more amino acid residue insertions, deletions, and/or substitutions. In certain embodiments, the sulfatase motif is within, or adjacent to, a region of an IgG3 heavy chain constant region corresponding to one or more of: 1) amino acids 1-6; 2) amino acids 13-22; 3) amino acids 33-37; 4) amino acids 43-61; 5) amino acid 71; 6) amino acids 78-80; 7) 87-91; 8) amino acids 97-106; 9) 111-115; 10) 147-167; 11) 173-177; 16) 185-187; 13) 195-203; 14) 210-218; 15) 226-227; 16) 238-239; 17) 246-248; 18) 255-261; 19) 267-275; 20) 282-291; 21) amino acids 303-307; 22) amino acids 313-320; 23) amino acids 324-333; 24) amino acids 350-352; 25) amino acids 359-365; and 26) amino acids 372-377; where the amino acid numbering is based on the numbering of the amino acid sequence set forth in SEQ ID NO: 92 (human IgG3) as depicted in FIG. 9B.

Exemplary surface-accessible loop regions of an IgG3 heavy chain include

1)
(SEQ ID NO: 93)
ASTKGP;
2)
(SEQ ID NO: 94)
PCSRSTSGGT;
3)
(SEQ ID NO: 95)
FPEPV;
4)
(SEQ ID NO: 96)
SGALTSGVHTFPAVLQSSG;
5)
V;
6)
TQT;
7)
(SEQ ID NO: 97)
HKPSN;
8)
(SEQ ID NO: 98)
RVELKTPLGD;
9)
(SEQ ID NO: 99)
CPRCPKP;
10)
(SEQ ID NO: 100)
PKSCDTPPPCPRCPAPELLGG;
11)
(SEQ ID NO: 101)
FPPKP;
12)
RTP;
13)
(SEQ ID NO: 102)
DVSHEDPEV;
14)
(SEQ ID NO: 103)
DGVEVHNAK;
15)
YN;
16)
VL;
17)
GKE;
18)
(SEQ ID NO: 104)
NKALPAP;
19)
(SEQ ID NO: 105)
SKTKGQPRE;
20)
P(SEQ ID NO: 106)
PSREEMTKN;
21)
(SEQ ID NO: 107)
YPSDI;
22)
(SEQ ID NO: 108)
SSGQPENN;
23)
(SEQ ID NO: 109)
TPPMLDSDGS;
24)
GNI;
25)
(SEQ ID NO: 110)
HEALHNR;
and
26)
(SEQ ID NO: 111)
SLSPGK.

In some instances, a target immunoglobulin amino acid sequence is modified to include a sulfatase motif as described above, where the modification includes one or more amino acid residue insertions, deletions, and/or substitutions. In certain embodiments, the sulfatase motif is within, or adjacent to, a region of an IgG4 heavy chain constant region corresponding to one or more of: 1) amino acids 1-5; 2) amino acids 12-23; 3) amino acids 32-36; 4) amino acids 42-53; 5) amino acids 57-62; 6) amino acids 68-70; 7) amino acids 77-79; 8) amino acids 86-88; 9) amino acids 94-95; 10) amino acids 101-102; 11) amino acids 108-118; 12) amino acids 122-126; 13) amino acids 134-136; 14) amino acids 144-152; 15) amino acids 159-167; 16) amino acids 175-176; 17) amino acids 185-186; 18) amino acids 196-198; 19) amino acids 205-211; 20) amino acids 217-226; 21) amino acids 232-241; 22) amino acids 253-257; 23) amino acids 264-265; 24) 269-270; 25) amino acids 274-283; 26) amino acids 300-303; 27) amino acids 399-417; where the amino acid numbering is based on the numbering of the amino acid sequence set forth in SEQ ID NO: 112 (human IgG4) as depicted in FIG. 9B.

Exemplary surface-accessible loop regions of an IgG4 heavy chain include

1)
(SEQ ID NO: 113)
STKGP;
2)
(SEQ ID NO: 114)
PCSRSTSESTAA;
3)
(SEQ ID NO: 115)
FPEPV;
4)
(SEQ ID NO: 116)
SGALTSGVHTFP;
5)
(SEQ ID NO: 117)
QSSGLY;
6)
VTV;
7)
TKT;
8)
HKP;
9)
DK;
10)
YG;
11)
(SEQ ID NO: 118)
CPAPEFLGGPS;
12)
(SEQ ID NO: 119)
FPPKP;
13)
RTP;
14)
(SEQ ID NO: 120)
DVSQEDPEV;
15)
(SEQ ID NO: 121)
DGVEVHNAK;
16)
FN;
17)
VL;
18)
GKE;
19)
(SEQ ID NO: 122)
NKGLPSS;
20)
(SEQ ID NO: 123)
SKAKGQPREP;
21)
(SEQ ID NO: 124)
PPSQEEMTKN;
22)
(SEQ ID NO: 125)
YPSDI;
23)
NG;
24)
NN;
25)
(SEQ ID NO: 126)
TPPVLDSDGS;
26)
(SEQ ID NO: 127)
GNVF;
27)
(SEQ ID NO: 128)
HEALHNHYTQKSLSLSLGK.

In some instances, a target immunoglobulin amino acid sequence is modified to include a sulfatase motif as described above, where the modification includes one or more amino acid residue insertions, deletions, and/or substitutions. In certain embodiments, the sulfatase motif is within, or adjacent to, a region of an IgA heavy chain constant region corresponding to one or more of: 1) amino acids 1-13; 2) amino acids 17-21; 3) amino acids 28-32; 4) amino acids 44-54; 5) amino acids 60-66; 6) amino acids 73-76; 7) amino acids 80-82; 8) amino acids 90-91; 9) amino acids 123-125; 10) amino acids 130-133; 11) amino acids 138-142; 12) amino acids 151-158; 13) amino acids 165-174; 14) amino acids 181-184; 15) amino acids 192-195; 16) amino acid 199; 17) amino acids 209-210; 18) amino acids 222-245; 19) amino acids 252-256; 20) amino acids 266-276; 21) amino acids 293-294; 22) amino acids 301-304; 23) amino acids 317-320; 24) amino acids 329-353; where the amino acid numbering is based on the numbering of the amino acid sequence set forth in SEQ ID NO: 129 (human IgA) as depicted in FIG. 9B.

Exemplary surface-accessible loop regions of an IgA heavy chain include

1)
(SEQ ID NO: 130)
ASPTSPKVFPLSL;
2)
(SEQ ID NO: 131)
QPDGN;
3)
(SEQ ID NO: 132)
VQGFFPQEPL;
4)
(SEQ ID NO: 133)
SGQGVTARNFP;
5)
(SEQ ID NO: 134)
SGDLYTT;
6)
(SEQ ID NO: 135)
PATQ;
7)
GKS;
8)
YT;
9)
CHP;
10)
(SEQ ID NO: 136)
HRPA;
11)
(SEQ ID NO: 137)
LLGSE;
12)
(SEQ ID NO: 138)
GLRDASGV;
13)
(SEQ ID NO: 139)
SSGKSAVQGP;
14)
(SEQ ID NO: 140)
GCYS;
15)
(SEQ ID NO: 141)
CAEP;
16)
PE;
17)
(SEQ ID NO: 142)
SGNTFRPEVHLLPPPSEELALNEL;
18)
(SEQ ID NO: 143)
ARGFS;
19)
(SEQ ID NO: 144)
QGSQELPREKY;
20)
AV;
21)
(SEQ ID NO: 145)
AAED;
22)
(SEQ ID NO: 146)
HEAL;
and
23)
(SEQ ID NO: 147)
IDRLAGKPTHVNVSVVMAEVDGTCY.

A sulfatase motif can be provided within or adjacent one or more of these amino acid sequences of such modification sites of an Ig heavy chain. For example, an Ig heavy chain polypeptide amino acid sequence can be modified (e.g., where the modification includes one or more amino acid residue insertions, deletions, and/or substitutions) at one or more of these amino acid sequences to provide a sulfatase motif adjacent and N-terminal and/or adjacent and C-terminal to these modification sites. Alternatively or in addition, an Ig heavy chain polypeptide amino acid sequence can be modified (e.g., where the modification includes one or more amino acid residue insertions, deletions, and/or substitutions) at one or more of these amino acid sequences to provide a sulfatase motif between any two residues of the Ig heavy chain modifications sites. In some embodiments, an Ig heavy chain polypeptide amino acid sequence may be modified to include two motifs, which may be adjacent to one another, or which may be separated by one, two, three, four or more (e.g., from about 1 to about 25, from about 25 to about 50, or from about 50 to about 100, or more, amino acids. Alternatively or in addition, where a native amino acid sequence provides for one or more amino acid residues of a sulfatase motif sequence, selected amino acid residues of the modification sites of an Ig heavy chain polypeptide amino acid sequence can be modified (e.g., where the modification includes one or more amino acid residue insertions, deletions, and/or substitutions) so as to provide a sulfatase motif at the modification site.

The amino acid sequence of a surface-accessible loop region can thus be modified to provide a sulfatase motif, where the modifications can include insertions, deletions, and/or substitutions. For example, where the modification is in a CH1 domain, the surface-accessible loop region can have the amino acid sequence NSGALTSG (SEQ ID NO: 148), and the aldehyde-tagged sequence can be, e.g., NSGALCTPSRG (SEQ ID NO: 149), e.g., where the “TS” residues of the NSGALTSG (SEQ ID NO: 150) sequence are replaced with “CTPSR,” (SEQ ID NO: 151) such that the sulfatase motif has the sequence LCTPSR (SEQ ID NO: 152). As another example, where the modification is in a CH2 domain, the surface-accessible loop region can have the amino acid sequence NKALPAP (SEQ ID NO: 153), and the aldehyde-tagged sequence can be, e.g., NLCTPSRAP (SEQ ID NO: 154), e.g., where the “KAL” residues of the NKALPAP (SEQ ID NO: 155) sequence are replaced with “LCTPSR,” (SEQ ID NO: 156) such that the sulfatase motif has the sequence LCTPSR (SEQ ID NO: 157). As another example, where the modification is in a CH2/CH3 domain, the surface-accessible loop region can have the amino acid sequence KAKGQPR (SEQ ID NO: 158), and the aldehyde-tagged sequence can be, e.g., KAKGLCTPSR (SEQ ID NO: 159), e.g., where the “GQP” residues of the KAKGQPR (SEQ ID NO: 160) sequence are replaced with “LCTPS,” (SEQ ID NO: 161) such that the sulfatase motif has the sequence LCTPSR (SEQ ID NO: 162).

As noted above, an isolated aldehyde-tagged anti-TACSTD2 Ig polypeptide can comprise a light chain constant region amino acid sequence modified to include a sulfatase motif as described above, where the sulfatase motif is in or adjacent a surface-accessible loop region of the Ig polypeptide light chain constant region.

In some instances, a target immunoglobulin amino acid sequence is modified to include a sulfatase motif as described above, where the modification includes one or more amino acid residue insertions, deletions, and/or substitutions. In certain embodiments, the sulfatase motif is within, or adjacent to, a region of an Ig light chain constant region corresponding to one or more of: 1) amino acids 130-135; 2) amino acids 141-143; 3) amino acid 150; 4) amino acids 162-166; 5) amino acids 163-166; 6) amino acids 173-180; 7) amino acids 186-194; 8) amino acids 211-212; 9) amino acids 220-225; 10) amino acids 233-236; wherein the amino acid numbering is based on the amino acid numbering of human kappa light chain as depicted in FIG. 9C. In some instances, a target immunoglobulin amino acid sequence is modified to include a sulfatase motif as described above, where the modification includes one or more amino acid residue insertions, deletions, and/or substitutions. In certain embodiments, the sulfatase motif is within, or adjacent to, a region of an Ig light chain constant region corresponding to one or more of: 1) amino acids 1-6; 2) amino acids 12-14; 3) amino acid 21; 4) amino acids 33-37; 5) amino acids 34-37; 6) amino acids 44-51; 7) amino acids 57-65; 8) amino acids 83-83; 9) amino acids 91-96; 10) amino acids 104-107; where the amino acid numbering is based on SEQ ID NO: 163 (human kappa light chain) as depicted in FIG. 9C.

Exemplary surface-accessible loop regions of an Ig light chain (e.g., a human kappa light chain) include: 1) RTVAAP (SEQ ID NO: 164); 2) PPS; 3) Gly (see, e.g., Gly at position 150 of the human kappa light chain sequence depicted in FIG. 9C); 4) YPREA (SEQ ID NO: 165); 5) PREA (SEQ ID NO: 166); 6) DNALQSGN (SEQ ID NO: 167); 7) TEQDSKDST (SEQ ID NO: 168); 8) HK; 9) HQGLSS (SEQ ID NO: 169); and 10) RGEC (SEQ ID NO: 170).

Exemplary surface-accessible loop regions of an Ig lambda light chain include QPKAAP (SEQ ID NO: 171), PPS, NK, DFYPGAV (SEQ ID NO: 172), DSSPVKAG (SEQ ID NO: 173), TTP, SN, HKS, EG, and APTECS (SEQ ID NO: 174).

In some instances, a target immunoglobulin amino acid sequence is modified to include a sulfatase motif as described above, where the modification includes one or more amino acid residue insertions, deletions, and/or substitutions. In certain embodiments, the sulfatase motif is within, or adjacent to, a region of a rat Ig light chain constant region corresponding to one or more of: 1) amino acids 1-6; 2) amino acids 12-14; 3) amino acids 121-22; 4) amino acids 31-37; 5) amino acids 44-51; 6) amino acids 55-57; 7) amino acids 61-62; 8) amino acids 81-83; 9) amino acids 91-92; 10) amino acids 102-105; wherein the amino acid numbering is based on the amino acid numbering of rat light chain as set forth in SEQ ID NO: 175 as depicted in FIG. 9C.

In some cases, a sulfatase motif is introduced into the CH1 region of an anti-TACSTD2 heavy chain constant region. In some cases, a sulfatase motif is introduced at or near (e.g., within 1 to 10 amino acids of) the C-terminus of an anti-TACSTD2 heavy chain. In some cases, a sulfatase motif is introduced in the light-chain constant region.

In some cases, a sulfatase motif is introduced into the CH1 region of an anti-TACSTD2 heavy chain constant region, e.g., within amino acids 121-219 of the IgG1 heavy chain amino acid sequence. For example, in some cases, a sulfatase motif is introduced into the amino acid sequence: ASTKGPSVFPLAPSSKSTSGGTAALGCLVKDYFPEPVTVSWNSGALTSGVHTFPAVLQSS GLYSLSSVVTVPSSSLGTQTYICNVNHKPSNTKVDKKVE (SEQ ID NO: 176). For example, in some of these embodiments, the amino acid sequence GALTSGVH (SEQ ID NO: 177) is modified to GALCTPSRGVH (SEQ ID NO: 178), where the sulfatase motif is LCTPSR (SEQ ID NO: 179).

In some cases, a sulfatase motif is introduced at or near the C-terminus of an anti-TACSTD2 heavy chain, e.g., the sulfatase motifs introduced within 1 amino acid, 2 amino acids (aa), 3 aa, 4 aa, 5 aa, 6 aa, 7 aa, 8 aa, 9 aa, or 10 aa the C-terminus of an anti-TACSTD2 heavy chain. As one non-limiting example, the C-terminal lysine residue of an anti-TACSTD2 heavy chain can be replaced with the amino acid sequence SLCTPSRGS (SEQ ID NO: 180).

In some cases, a sulfatase motif is introduced into the constant region of a light chain of an anti-TACSTD2 antibody. As one non-limiting example, in some cases, a sulfatase motif is introduced into the constant region of a light chain of an anti-TACSTD2 antibody, where the sulfatase motif is C-terminal to KVDNAL (SEQ ID NO: 181), and/or is N-terminal to QSGNSQ (SEQ ID NO: 182). For example, in some cases, the sulfatase motif is LCTPSR (SEQ ID NO: 183), and the anti-TACSTD2 light chain comprises the amino acid sequence KVDNALLCTPSRQSGNSQ (SEQ ID NO: 184).

### Muc-1 Antibody

As noted above, according to the methods of this invention, a subject can be administered a conjugate that comprises, as substituent W¹ an antibody. In certain embodiments, of the methods described herein, the subject has a disorder exhibited by a MUC1-positive cell, e.g., a cancerous MUC1-positive cell or an autoreactive MUC1-positive cell. In certain embodiments of the methods disclosed herein, the antibody W² can be an anti-Muc-1 antibody, where the amino acid sequence of the anti-Muc-1 antibody has been modified to include a 2-formylglycine (fGly) residue. As used herein, amino acids may be referred to by their standard name, their standard three letter abbreviation and/or their standard one letter abbreviation, such as: Alanine or Ala or A; Cysteine or Cys or C; Aspartic acid or Asp or D; Glutamic acid or Glu or E; Phenylalanine or Phe or F; Glycine or Gly or G; Histidine or His or H; Isoleucine or Ile or I; Lysine or Lys or K; Leucine or Leu or L; Methionine or Met or M; Asparagine or Asn or N; Proline or Pro or P; Glutamine or Gln or Q; Arginine or Arg or R; Serine or Ser or S; Threonine or Thr or T; Valine or Val or V; Tryptophan or Trp or W; and Tyrosine or Tyr or Y.

According to some embodiments, an antibody of the present disclosure specifically binds to MUC1 and competes for binding to MUC1 with an antibody comprising:

a variable heavy chain (VH) chain comprising heavy chain CDRs1-3 (HCDRs1-3) of a V_{H} chain having the sequence:

(SEQ ID NO: 9)
EVQLVQSGAEVKKPGATVKISCKVSGYTFTDHTMHWIKQRPGKGL
EWMGYFYPRDDSTNYNEKFKGRVTLTADKSTDTAYMELSSLRSED
TAVYYCARGLRYALDYWGQGTLVTVSS;

and
a variable light chain (VL) chain comprising light chain CDRs1-3 (LCDRs1-3) of a VL chain having the sequence:

(SEQ ID NO: 7)
EIVLTQSPATLSLSPGERATLSCRASSSVSSSYLYWYQQKPGQAP
RLWIYGTSNLASGVPARFSGSGSGTDYTLTISSLEPEDAAVYYCH
QYAWSPPTFGQGTKLEIK;
(SEQ ID NO: 1)
EIVLTQSPATLSLSPGERATLSCRASSSVGSSNLYWYQQKPGQAP
RLWIYRSTKLASGVPARFSGSGSGTDYTLTISSLEPEDAAVYYCH
QYRWSPPTFGQGTKLEIK;
or
(SEQ ID NO: 2)
EIVLTQSPATLSLSPGERATLSCRASSSVSSSYLYWYQQKPGQAP
RLWIIGTSNLASGVPARFSGSGSGTDYTLTISSLEPEDAAVYYCH
QYSWSPPTFGQGTKLEIK.

Any suitable approach for determining whether a first antibody competes with a second antibody for binding to MUC1 may be employed. Whether a first antibody “competes with” a second antibody for binding to MUC1 may be readily determined using competitive binding assays known in the art. Competing antibodies may be identified, for example, via an antibody competition assay. For example, a sample of a first antibody can be bound to a solid support. Then, a sample of a second antibody suspected of being able to compete with such first antibody is added. One of the two antibodies is labelled. If the labeled antibody and the unlabeled antibody bind to separate and discrete sites on MUC1, the labeled antibody will bind to the same level whether or not the suspected competing antibody is present. However, if the sites of interaction are identical or overlapping, the unlabeled antibody will compete, and the amount of labeled antibody bound to MUC1 will be lowered. If the unlabeled antibody is present in excess, very little, if any, labeled antibody will bind.

For purposes of the present disclosure, competing antibodies are those that decrease the binding of an antibody to MUC1 by about 50% or more, about 60% or more, about 70% or more, about 80% or more, about 85% or more, about 90% or more, about 95% or more, or about 99% or more. Details of procedures for carrying out such competition assays are well known in the art. Such assays can be made quantitative by using purified antibodies. A standard curve may be established by titrating one antibody against itself, i.e., the same antibody is used for both the label and the competitor. The capacity of an unlabeled competing antibody to inhibit the binding of the labeled antibody to the antigen may be titrated. The results may be plotted, and the concentrations necessary to achieve the desired degree of binding inhibition may be compared.

According to some embodiments, an antibody of the present disclosure specifically binds to MUC1 and comprises:
a variable heavy chain (VH) chain comprising heavy chain CDRs1-3 (HCDRs1-3) of a V_{H} chain having the sequence:

(SEQ ID NO: 9)
EVQLVQSGAEVKKPGATVKISCKVSGYTFTDHTMHWIKQRPGKGL
EWMGYFYPRDDSTNYNEKFKGRVTLTADKSTDTAYMELSSLRSED
TAVYYCARGLRYALDYWGQGTLVTVSS;

a variable light chain (VL) chain comprising light chain CDRs1-3 (LCDRs1-3) of a VL chain having the sequence:

(SEQ ID NO: 7)
EIVLTQSPATLSLSPGERATLSCRASSSVSSSYLYWYQQKPGQAP
RLWIYGTSNLASGVPARFSGSGSGTDYTLTISSLEPEDAAVYYCH
QYAWSPPTFGQGTKLEIK;
(SEQ ID NO: 1)
EIVLTQSPATLSLSPGERATLSCRASSSVGSSNLYWYQQKPGQAP
RLWIYRSTKLASGVPARFSGSGSGTDYTLTISSLEPEDAAVYYCH
QYRWSPPTFGQGTKLEIK;
or
(SEQ ID NO: 2)
EIVLTQSPATLSLSPGERATLSCRASSSVSSSYLYWYQQKPGQAP
RLWIIGTSNLASGVPARFSGSGSGTDYTLTISSLEPEDAAVYYCH
QYSWSPPTFGQGTKLEIK

The HCDRs1-3 and LCDRs1-3 may be as defined by Chothia, Kabat, or IMT nomenclature. The HCDRs1-3 of the anti-MUC1 antibodies disclosed herein as defined per the listed nomenclatures may be as follows:

TABLE 5

| Anti-MUC1 | | | |
|---|---|---|---|
| Antibody | Chothia | Kabat | IMGT |
| HCDR1 | GYTFTDH | DHTMH | GYTFTDHT |
| | (SEQ ID | (SEQ ID | (SEQ ID |
| | NO: 8) | NO: 10) | NO: 38) |
| HCDR2 | YPRDDS | YFYPRDDST | FYPRDDST |
| | (SEQ ID | NYNEKFKG | (SEQ ID |
| | NO: 4) | (SEQ ID | NO: 73) |
| | | NO: 11) | |
| HCDR3 | GLRYALDY | GLRYALDY | ARGLRYALDY |
| | (SEQ ID | (SEQ ID | (SEQ ID |
| | NO: 5) | NO: 5) | NO: 92) |

The LCDRs1-3 of the anti-MUC1 antibodies disclosed herein may be as defined per the nomenclatures listed in Tables 6-8.

TABLE 6

| Anti-MUC1 | Chothia and | |
|---|---|---|
| Antibody | Kabat | IMGT |
| LCDR1 | RASSSVSSSYLY | SSVSSSY |
| | (SEQ ID NO: 6) | SEQ ID NO: 37) |
| LCDR2 | GTSNLAS | GT |
| | (SEQ ID NO: 12) | |
| LCDR3 | HQYAWSPPT | HQYAWSPPT |
| | (SEQ ID NO: 13) | (SEQ ID NO: 13) |

TABLE 7

| Anti-MUC1 | Chothia and | |
|---|---|---|
| Antibody | Kabat | IMGT |
| LCDR1 | RASSSVGSSNLY | SSVGSSN |
| | (SEQ ID NO: 14) | (SEQ ID NO: |
| | | 112) |
| LCDR2 | RSTKLAS | RS |
| | (SEQ ID NO: 15) | |
| LCDR3 | HQYRWSPPT | HQYRWSPPT |
| | (SEQ ID NO: 16) | (SEQ ID NO: 16) |

TABLE 8

| Anti-MUC1 | Chothia and | |
|---|---|---|
| Antibody | Kabat | IMGT |
| LCDR1 | RASSSVSSSYLY | SSVSSSY |
| | (SEQ ID NO: 6) | (SEQ ID NO: 37) |
| LCDR2 | GTSNLAS | GT |
| | (SEQ ID NO: 12) | |
| LCDR3 | HQYSWSPPT | HQYSWSPPT |
| | (SEQ ID NO: 17) | (SEQ ID NO: 17) |

In certain embodiments, the V_{H} chain of an anti-MUC1 antibody comprises the HCDRs1-3 as set forth herein and the V_{L} chain of the anti-MUC1 antibody comprises LCDRs1-3, wherein:
the LCDR1 comprises the amino acid sequence RASSSVG/SSSYLY (SEQ ID NO: 45);
the LCDR2 comprises the amino acid sequence G/RT/SS/TN/KLAS (SEQ ID NO: 46);
the LCDR3 comprises the amino acid sequence HQYA/R/SWSPPT (SEQ ID NO: 47), as per Kabat definition.

In certain embodiments, the V_{H} chain of an anti-MUC1 antibody comprises the HCDRs1-3 as set forth herein and comprises an amino acid sequence having 80% or greater, 85% or greater, 90% or greater, 95% or greater, 99% or greater, or 100% sequence identity to the amino acid sequence set forth in SEQ ID NO: 9. In certain embodiments, any amino acid differences between the V_{H} chain of an anti-MUC1 antibody of the present disclosure and SEQ ID NO: 9 may be limited to regions outside of the CDRs, e.g., in one or more of the framework regions (FR), e.g., FR1, FR2, FR3, and/or FR4.

In certain embodiments, the V_{L} chain of an anti-MUC1 antibody comprises the LCDRs1-3 as set forth herein in Table 6 and comprises an amino acid sequence having 80% or greater, 85% or greater, 90% or greater, 95% or greater, 99% or greater, or 100% sequence identity to the amino acid sequence set forth in SEQ ID NO: 7.

In certain embodiments, the V_{L} chain of an anti-MUC1 antibody comprises the LCDRs1-3 as set forth herein in Table 7 and comprises an amino acid sequence having 80% or greater, 85% or greater, 90% or greater, 95% or greater, 99% or greater, or 100% sequence identity to the amino acid sequence set forth in SEQ ID NO: 1.

In certain embodiments, the V_{L} chain of an anti-MUC1 antibody comprises the LCDRs1-3 as set forth herein in Table 8 and comprises an amino acid sequence having 80% or greater, 85% or greater, 90% or greater, 95% or greater, 99% or greater, or 100% sequence identity to the amino acid sequence set forth in SEQ ID NO: 2.

In certain embodiments, any amino acid differences between the V_{L} chain of an anti-MUC1 antibody of the present disclosure and SEQ ID NO: 7, 1, and 2 may be limited to regions outside of the CDRs, e.g., in one or more of the framework regions (FR), e.g., FR1, FR2, FR3, and/or FR4.

In certain embodiments, an anti-MUC1 antibody of the present disclosure can comprise: a) a heavy chain comprising a V_{H} region having the amino acid sequence set forth in SEQ ID NO: 9; and a light chain comprising the V_{L} region having the amino acid sequence set forth in SEQ ID NO: 7, 1, or 2.

The anti-MUC1 antibodies of the present disclosure may bind to cancerous tissue and may show no binding (e.g., insignificant binding as measured by immunohistochemistry or binding undetectable by immunohistochemistry) to normal tissue. For example, the anti-MUC1 antibodies described herein may bind to human gastric, breast, and/or lung tissue that have cancerous cells while showing no detectable binding to human gastric, breast, and/or lung tissue that do not have cancerous cells.

In certain embodiments, the V_{H} region of an anti-MUC1 antibody of the present disclosure is encoded by a nucleic acid having at least 50%, at least 60%, at least 70%, at least 80%, at least 90%, at least 95%, or a 100% sequence identity to the nucleic acid sequence:

(SEQ ID NO: 23)
GAGGTCCAGCTGGTACAGTCTGGGGCTGAGGTGAAGAAGCCTGGG
GCTACAGTGAAAATCTCCTGCAAGGTTTCTGGATACACCTTCACC
GACCATACCATGCACTGGATCAAACAGCGACCTGGAAAAGGGCTT
GAGTGGATGGGATACTTCTACCCTAGAGATGATTCCACAAATTAC
AACGAGAAGTTCAAGGGCAGAGTCACCCTTACCGCGGACAAATCT
ACAGACACAGCCTACATGGAGCTGAGCAGCCTGAGATCTGAGGAC
ACGGCCGTGTATTACTGTGCGCGTGGTCTTCGATACGCTCTTGAC
TACTGGGGCCAAGGAACCCTGGTCACCGTCTCCTCA

In certain embodiments, the V_{L} region of an anti-MUC1 antibody of the present disclosure is encoded by a nucleic acid having at least 50%, at least 60%, at least 70%, at least 80%, at least 90%, at least 95%, or a 100% sequence identity to the nucleic acid sequence:

(SEQ ID NO: 42)
GAAATTGTGTTGACACAGTCTCCAGCCACCCTGTCTTTGTCTCCA
GGGGAAAGAGCCACCCTCTCCTGCAGGGCCAGTTCAAGTGTTAGC
AGCAGCTACTTATACTGGTACCAGCAGAAACCTGGCCAGGCTCCC
AGGCTCTGGATCTATGGTACCTCCAACCTTGCCTCCGGCGTCCCA
GCAAGGTTCAGTGGCAGTGGGTCTGGGACAGACTACACTCTCACC
ATCAGCTCCCTGGAGCCTGAAGATGCGGCAGTTTATTACTGTCAC
CAATACGCCTGGTCCCCGCCGACGTTCGGCCAAGGGACCAAGTTG
GAAATCAAA;
(SEQ ID NO: 43)
GAAATTGTGTTGACACAGTCTCCAGCCACCCTGTCTTTGTCTCCA
GGGGAAAGAGCCACCCTCTCCTGCAGGGCCAGTTCAAGTGTTGGC
AGCAGCAACTTATACTGGTACCAGCAGAAACCTGGCCAGGCTCCC
AGGCTCTGGATCTATAGGTCCACCAAACTTGCCTCCGGCGTCCCA
GCAAGGTTCAGTGGCAGTGGGTCTGGGACAGACTACACTCTCACC
ATCAGCTCCCTGGAGCCTGAAGATGCGGCAGTTTATTACTGTCAC
CAATACAGATGGTCCCCGCCGACGTTCGGCCAAGGGACCAAGTTG
GAAATCAAA;
or
(SEQ ID NO: 44)
GAAATTGTGTTGACACAGTCTCCAGCCACCCTGTCTTTGTCTCCA
GGGGAAAGAGCCACCCTCTCCTGCAGGGCCAGTTCAAGTGTTAGC
AGCAGCTACTTATACTGGTACCAGCAGAAACCTGGCCAGGCTCCC
AGGCTCTGGATCATTGGTACCTCCAACCTTGCCTCCGGCGTCCCA
GCAAGGTTCAGTGGCAGTGGGTCTGGGACAGACTACACTCTCACC
ATCAGCTCCCTGGAGCCTGAAGATGCGGCAGTTTATTACTGTCAC
CAATACTCCTGGTCCCCGCCGACGTTCGGCCAAGGGACCAAGTTG
GAAATCAAA.

In certain aspects of the invention, the antibody specifically binds a MUC1 polypeptide, where the epitope comprises amino acid residues within a human MUC1 antigen comprising the amino acid sequence set forth in SEQ ID NO: 24:

(SEQ ID NO: 24)
MTPGTQSPFFLLLLLTVLTVVTGSGHASSTPGGEKETSATQRSSV
PSSTEKNAVSMTSSVLSSHSPGSGSSTTQGQDVTLAPATEPASGS
AATWGQDVTSVPVTRPALGSTTPPAHDVTSAPDNKPAPGSTAPPA
HGVTSAPDTRPAPGSTAPPAHGVTSAPDTRPAPGSTAPPAHGVTS
APDTRPAPGSTAPPAHGVTSAPDTRPAPGSTAPPAHGVTSAPDTR
PAPGSTAPPAHGVTSAPDTRPAPGSTAPPAHGVTSAPDTRPAPGS
TAPPAHGVTSAPDTRPAPGSTAPPAHGVTSAPDTRPAPGSTAPPA
HGVTSAPDTRPAPGSTAPPAHGVTSAPDTRPAPGSTAPPAHGVTS
APDTRPAPGSTAPPAHGVTSAPDTRPAPGSTAPPAHGVTSAPDTR
PAPGSTAPPAHGVTSAPDTRPAPGSTAPPAHGVTSAPDTRPAPGS
TAPPAHGVTSAPDTRPAPGSTAPPAHGVTSAPDTRPAPGSTAPPA
HGVTSAPDTRPAPGSTAPPAHGVTSAPDTRPAPGSTAPPAHGVTS
APDTRPAPGSTAPPAHGVTSAPDTRPAPGSTAPPAHGVTSAPDTR
PAPGSTAPPAHGVTSAPDTRPAPGSTAPPAHGVTSAPDTRPAPGS
TAPPAHGVTSAPDTRPAPGSTAPPAHGVTSAPDTRPAPGSTAPPA
HGVTSAPDTRPAPGSTAPPAHGVTSAPDTRPAPGSTAPPAHGVTS
APDTRPAPGSTAPPAHGVTSAPDTRPAPGSTAPPAHGVTSAPDTR
PAPGSTAPPAHGVTSAPDTRPAPGSTAPPAHGVTSAPDTRPAPGS
TAPPAHGVTSAPDTRPAPGSTAPPAHGVTSAPDTRPAPGSTAPPA
HGVTSAPDTRPAPGSTAPPAHGVTSAPDTRPAPGSTAPPAHGVTS
APDTRPAPGSTAPPAHGVTSAPDTRPAPGSTAPPAHGVTSAPDNR
PALGSTAPPVHNVTSASGSASGSASTLVHNGTSARATTTPASKST
PFSIPSHHSDTPTTLASHSTKTDASSTHHSSVPPLTSSNHSTSPQ
LSTGVSFFFLSFHISNLQFNSSLEDPSTDYYQELQRDISEMFLQI
YKQGGFLGLSNIKFRPGSVVVQLTLAFREGTINVHDVETQFNQYK
TEAASRYNLTISDVSVSDVPFPFSAQSGAGVPGWGIALLVLVCVL
VALAIVYLIALAVCQCRRKNYGOLDIFPARDTYHPMSEYPTYHTH
GRYVPPSSTDRSPYEKVSAGNGGSSLSYTNPAVAATSANL

In certain embodiments, the MUC1 epitope bound by the anti-MUC1 antibodies disclosed herein is glycosylated. In certain embodiments, the MUC1 epitope bound by the anti-MUC1 antibodies disclosed herein is present on MUC1 expressed by epipulmonary adenocarcinoma cell lines and pulmonary epithelial cells.

In other embodiments, the subject antibody exhibits high affinity binding to MUC1. For example, a subject antibody binds to MUC1 with an affinity of at least about 10-7 M, at least about 10-8 M, at least about 10-9 M, at least about 10-10 M, at least about 10-11 M, or at least about 10-12 M, or greater than 10-12 M. A subject antibody binds to an epitope present on MUC1 with an affinity of from about 10-7 M to about 10-8 M, from about 10-8 M to about 10-9 M, from about 10-9 M to about 10-10 M, from about 10-10 M to about 10-11 M, or from about 10-11 M to about 10-12 M, or greater than 10-12 M.

An anti-MUC1 antibody of the present disclosure can in some cases induce apoptosis in a cell that expresses MUC1 on its cell surface.

Further, a “MUC1 antigen” or “MUC1 polypeptide” can comprises an amino acid sequence having at least about 75%, at least about 80%, at least about 90%, at least about 95%, at least about 98%, at least about 99%, or 100%, amino acid sequence identity to SEQ ID NO: 24.

In some embodiments, an anti-MUC1 antibody of the present disclosure may include one or more amino acid substitutions introduced in the Fc region. In some embodiments, the one or more of the amino acid substitutions may be at the positions 239, 298, 326, 330 and 332 in the Fc region. In some embodiments, an anti-MUC1 antibody of the present disclosure may include one or more of the following amino acid substitutions introduced in the Fc region: I332E; S239D/A330L/I332E; S239D/S298A/I332E; S239D/K326T/I332E; S239D/S298A/K326T/I332E; or S239D/A330L/I332E/D356E/L358M.

### Nectin-4 Antibody

As disclosed herein, according to the methods of this invention, a subject can be administered a conjugate of Formula (I) that comprise, as substituent W¹ an antibody. In certain embodiments, the antibody can be an anti-Nectin-4 antibody, where the amino acid sequence of the anti-Nectin-4 antibody has been modified to include a 2-formylglycine (fGly) residue.

The antibodies that bind to nectin-4 can be, for example, as described in WO 2018/226578, the disclosure of which are incorporated herein by reference in its entirety.

### Napi2B Antibody

As disclosed herein, according to the methods of this invention, a subject can be administered a conjugate of Formula (I) that comprise, as substituent W¹ an antibody. In certain embodiments, the antibody can be an anti-Nectin-4 antibody, where the amino acid sequence of the anti-Nectin-4 antibody has been modified to include a 2-formylglycine (fGly) residue.

The antibodies that bind to nectin-4 can be, for example, as described in WO 2017/160754, the disclosure of which are incorporated herein by reference in its entirety.

### Drugs for Conjugation of a Polypeptide

The present disclosure provides a method of reducing toxicity by using drug-polypeptide conjugates of Formula (I). Examples of drugs include small molecule drugs, such as a cancer chemotherapeutic agent. For example, where the polypeptide is an antibody (or fragment thereof) that has specificity for a tumor cell, the antibody can be modified as described herein to include a modified amino acid, which can be subsequently conjugated to a cancer chemotherapeutic agent, such as a microtubule affecting agents. In certain embodiments, the drug is a microtubule affecting agent that has antiproliferative activity, such as a maytansinoid. In certain embodiments, the drug is a maytansinoid, which as the following structure:

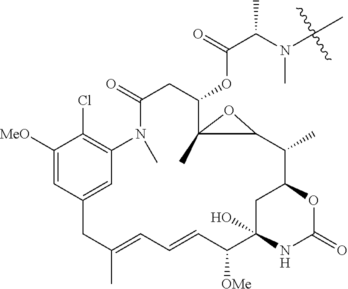

where [symbol] indicates the point of attachment between the maytansinoid and the linker in formula (I). By “point of attachment” is meant that the [symbol] symbol indicates the bond between the N of the maytansinoid and the linker in formula (I). For example, in the antibody-drug conjugate of formula (I), the drug is a maytansinoid, such as a maytansinoid of the structure above, where [symbol] indicates the point of attachment between the maytansinoid and the linker. In some instances, the maytansinoid structure shown above may be referred to as deacylmaytansine.

As described above, in certain embodiments, a linker can attach together (e.g., via one or more covalent bonds) the antibody and the drug of the antibody-drug conjugate of Formula (I) described herein.

In certain embodiments, the linker of the antibody-drug conjugate of Formula (I) has the following structure:

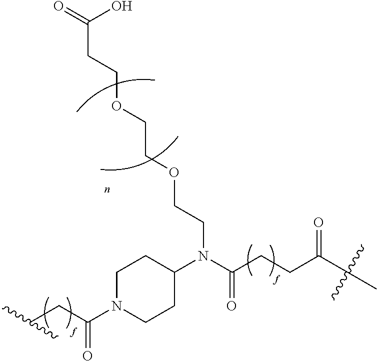

In certain embodiments of the linker structures depicted above, each f is independently 0 or an integer from 1 to 12; and n is 0 or an integer from 1 to 30. In certain embodiments of the linker structures depicted above, each f is independently 0, 1, 2, 3, 4, 5 or 6; and n is 0, 1, 2, 3, 4, 5 or 6. In certain embodiments of the linker structures depicted above, each f is 2 and n is 1. In the linker structure depicted above, the wavy lines [symbol] indicate the respective points of attachment between the linker and the hydrazinyl-indolyl coupling moiety and the linker (left-hand side wavy line) and the linker and the maytansinoid (right-hand side wavy line).

### Formulations

The conjugates of the present disclosure can be formulated in a variety of different ways. In general, where the conjugate is a antibody-drug conjugate, the conjugate is formulated in a manner compatible with the drug conjugated to the antibody, the condition to be treated, and the route of administration to be used.

In some embodiments, provided is a pharmaceutical composition that includes any of the conjugates of the present disclosure and a pharmaceutically-acceptable excipient.

The conjugate (e.g., antibody-drug conjugate) can be provided in any suitable form, e.g., in the form of a pharmaceutically acceptable salt, and can be formulated for any suitable route of administration, e.g., oral, topical or parenteral administration. Where the conjugate is provided as a liquid injectable (such as in those embodiments where they are administered intravenously or directly into a tissue), the conjugate can be provided as a ready-to-use dosage form, or as a reconstitutable storage-stable powder or liquid composed of pharmaceutically acceptable carriers and excipients.

Methods for formulating conjugates can be adapted from those readily available. For example, conjugates can be provided in a pharmaceutical composition comprising a therapeutically effective amount of a conjugate and a pharmaceutically acceptable carrier (e.g., saline). The pharmaceutical composition may optionally include other additives (e.g., buffers, stabilizers, preservatives, and the like). In some embodiments, the formulations are suitable for administration to a mammal, such as those that are suitable for administration to a human.

### Examples

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. By “average” is meant the arithmetic mean. Standard abbreviations may be used, e.g., bp, base pair(s); kb, kilobase(s); pl, picoliter(s); s or see, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### General Synthetic Procedures

Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

Compounds as described herein can be purified by any purification protocol known in the art, including chromatography, such as HPLC, preparative thin layer chromatography, flash column chromatography and ion exchange chromatography. Any suitable stationary phase can be used, including normal and reversed phases as well as ionic resins. In certain embodiments, the disclosed compounds are purified via silica gel and/or alumina chromatography. See, e.g., Introduction to Modern Liquid Chromatography, 2nd Edition, ed. L. R. Snyder and J. J. Kirkland, John Wiley and Sons, 1979; and Thin Layer Chromatography, ed E. Stahl, Springer-Verlag, New York, 1969.

During any of the processes for preparation of the subject compounds, it may be necessary and/or desirable to protect sensitive or reactive groups on any of the molecules concerned. This may be achieved by means of conventional protecting groups as described in standard works, such as J. F. W. McOmie, “Protective Groups in Organic Chemistry,” Plenum Press, London and New York 1973, in T. W. Greene and P. G. M. Wuts, “Protective Groups in Organic Synthesis,” Third edition, Wiley, New York 1999, in “The Peptides”; Volume 3 (editors: E. Gross and J. Meienhofer), Academic Press, London and New York 1981, in “Methoden der organischen Chemie,” Houben-Weyl, 4^{th} edition, Vol. 15/1, Georg Thieme Verlag, Stuttgart 1974, in H.-D. Jakubke and H. Jescheit, “Aminosauren, Peptide, Proteine,” Verlag Chemie, Weinheim, Deerfield Beach, and Basel 1982, and/or in Jochen Lehmann, “Chemie der Kohlenhydrate: Monosaccharide and Derivate,” Georg Thieme Verlag, Stuttgart 1974. The protecting groups may be removed at a convenient subsequent stage using methods known from the art.

The subject compounds can be synthesized via a variety of different synthetic routes using commercially available starting materials and/or starting materials prepared by conventional synthetic methods. A variety of examples of synthetic routes that can be used to synthesize the compounds disclosed herein are described in the schemes below.

### Example 1

### RED-106 Bioconjugation, Purification, and HPLC Analytics

Aldehyde-tagged antibodies (15 mg/mL) were conjugated to linker-payloads (8 mol. equivalents drug: antibody) for 72 h at 37° C. in 20 mM sodium citrate, 50 mM NaCl pH 5.5 containing 0.85% DMA. In some cases, to improve linker-payload solubility, additional DMA was added up to a maximum of 10% vol/vol. After conjugation, free drug was removed by using multiple rounds of dilution into 20 mM sodium citrate, 50 mM NaCl pH 5.5 and concentration using Amicon 0.5 mL 30 kD MWCO centrifugal filters (Millipore Sigma #UFC5030BK). To determine the DAR of the final product, ADCs were examined by analytical HIC or PLRP. The HIC column (Tosoh #14947) was run with mobile phase A: 1.5 M ammonium sulfate, 25 mM sodium phosphate pH 7.0, and mobile phase B: 25% isopropanol, 18.75 mM sodium phosphate pH 7.0. The PLRP column (Agilent #PL1912-1802) was run with mobile phase A: 0.1% trifluoroacetic acid in H₂O, and mobile phase B: 0.1% trifluoroacetic acid in CH3CN with the column heated to 80° C. To determine aggregation, samples were analyzed using analytical size exclusion chromatography (SEC; Tosoh #08541) with a mobile phase of 300 mM NaCl, 25 mM sodium phosphate pH 6.8, 5% isopropanol.

### Maleimide Conjugation of Untagged (Wild-Type) Antibodies

Antibodies (5 mg/mL) were reduced using 2.5 mol. equivalents of TCEP for 90 min at 37° C. in in PBS, pH 8.0, 1 mM DTPA. TCEP was removed and the protein was exchanged into PBS, pH 7.4, 1 mM DTPA using tangential flow filtration. Reduced antibody (3 mg/mL) was conjugated with 10 mol. equiv of maleimide-valcit-MMAE for 60 min on ice. Free drug was removed and final ADC was exchanged into PBS, pH 7.4 using tangential flow filtration.

### In Vitro Cytotoxicity Assays

Cell lines were plated on Day −1 in 96-well plates (Corning #3603) at a density of 4×103 cells/well in 100 μL of growth media and cultured overnight. On Day 0, serial dilution of test samples was performed in growth media at 6× the final concentration and 20 μL was added to the cells. After incubation at 37° C. with 5% CO2 for 5 days, viability was measured using Promega CellTiter-Glo® according to the manufacturer's recommendations.

### Non-GLP Rat Toxicology Study

Male Sprague-Dawley rats (8-9 wk old at study start, 5 animals/group) were dosed intravenously with either vehicle alone or with nectin-4 conjugates made using antibodies carrying the variable regions of the rat cross-reactive antibody, enfortumab. The tested ADCs were nectin-4 vedotin (dosed at 10 mg/kg), and nectin-4 CH1/CT RED-106 (dosed at either 10 or 20 mg/kg). Dosing occurred weekly for a total of 4 doses (days 1, 8, 15, and 22). Animals were observed for 7 days post last dose. Body weights were recorded four times/week. Blood was collected from all animals for clinical pathology on days 5, 12, 19, and 26, and for toxicokinetic analysis at 8 h and days 4 and 7 post-dose (for all doses). Clinical observations were conducted daily. The clinical observation scoring system scale ranged from 0 (normal) to 3 (severe) is shown in Table 2.

### Xenograft Studies

Methods: Female SCID Beige mice (8/group) were inoculated subcutaneously with 5 million NCI-H292 cells in PBS. Treatment began when the tumors reached an average of 121 mm3 (Day 1). For treatment, animals were dosed intravenously with vehicle alone, CAT-10⁻¹⁰⁶, Trodelvy, or DS-1062. The dosing schedule was designed to mirror the Trodelvy and DS-1062 clinical dosing schedules. ADCs were either dosed on Days 0, 7, 21 and 28 (Trodelvy and some CAT-10⁻¹⁰⁶ groups) or on Days 0 and 21 (DS-1062 and one CAT-10⁻¹⁰⁶ group). The animals were monitored twice weekly for body weight and tumor size. Animals were euthanized when tumors reached 2000 mm3 or body weight loss exceeded 15%. NCI-H292 induces cachexia in mice so animals with uncontrolled or poorly controlled tumor growth exhibited body weight loss.

### Non-Human Primate Toxicology Study (Non-GLP)

Methods: Female cynomolgus monkeys were given four doses of 1.5, 3, or 5 mg/kg of CAT-10⁻¹⁰⁶ on Days 1, 8, 22, and 29, followed by a 14 day observation period (Table 9). Clinical observations were conducted daily; body weight was measured twice predose and then weekly. Dose site dermal observations were conducted once predose and on dosing days. Clinical pathology (hematology, coagulation, chemistry) was assessed twice predose and on Days 5, 8 (pre-dose), 12, 18, 22 (pre-dose), 26, 29, and 32, and for recovery animals on day 43 (recovery day 12). Urine analysis was conducted once predose, day 32, and for recovery animals on day 43 (recovery day 12).

TABLE 9
Tumor-Associated Calcium Signal Transducer 2 (TACSTD2)
ADC non-GLP NHP Toxicology Study Design.

| Group | # Animals Terminal | # Animals Recovery | Dose (mg/kg) |
|---|---|---|---|
| 1 | 3 | 2 | Vehicle |
| 2 | 3 | 0 | 1.5 |
| 3 | 3 | 0 | 3 |
| 4 | 3 | 2 | 5 |

Female cynomolgus monkeys were dosed i.v. on Days 1, 8, 22 and 29, with terminal sacrifice on Day 32 and a recovery period through Day 43.

### Toxicokinetic Sample Analysis

Methods: Total antibody and total ADC concentrations were quantified by ELISA as previously described and diagrammed in the figure. For total antibody, conjugates were captured with an anti-human IgG-specific antibody and detected with an HRP-conjugated anti-human Fc-specific antibody. For total ADC, conjugates were captured with an anti-human Fab-specific antibody and detected with a mouse anti-maytansine primary antibody, followed by an HRP-conjugated anti-mouse IgG-subclass 1-specific secondary antibody. Bound secondary antibody was detected using Ultra TMB One-Step ELISA substrate (Thermo Fisher). After quenching the reaction with sulfuric acid, signals were read by taking the absorbance at 450 nm on a Molecular Devices Spectra Max M5 plate reader equipped with SoftMax Pro software. Data were analyzed using GraphPad Prism and Microsoft Excel software.

### Example 2: In-Vitro Cytotoxicity Assay Testing Anti-Nectin-4 ADCs

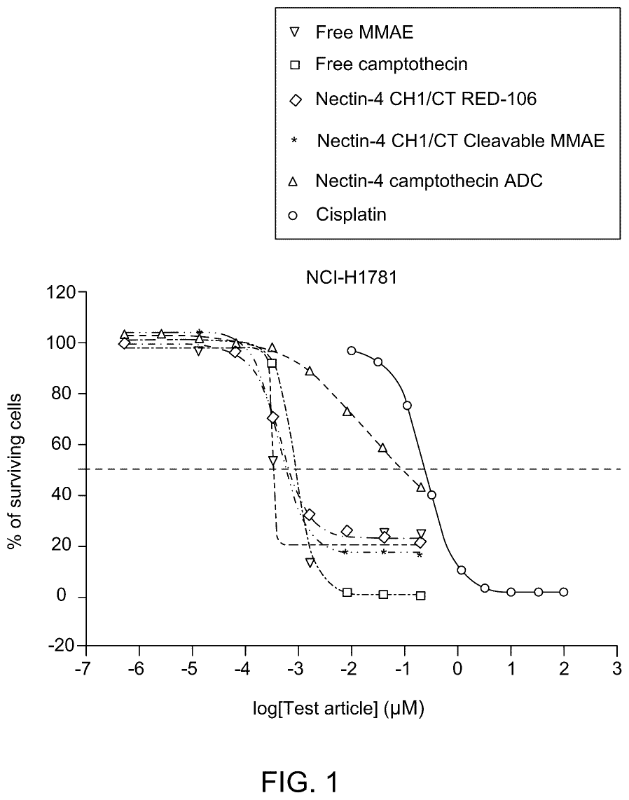

Methods: Free payloads (MMAE and camptothecin) and nectin-4 ADCs carrying either MMAE, camptothecin, or maytansine were produced and tested in an in vitro cytotoxicity assay against the nectin-4-expressing cell line, NCI-H1781 (FIG. 1).

Results: In vitro potency of RED-106 and MMAE conjugates were similar against the target cell line, as shown in FIG. 1.

### Example 3: Comparative Studies of Non-GLP Rat Toxicity Study and Toxicokinetics Using Anti-Nectin-4 Carrying RED-106 or Anti-Nectin-4 Carrying Vedotin Linker Payloads

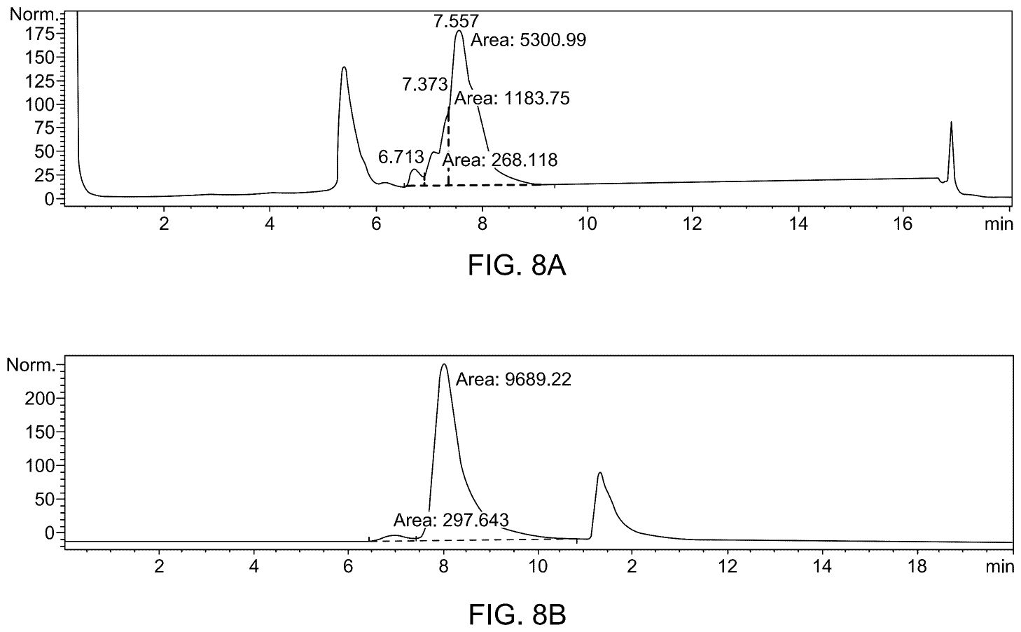

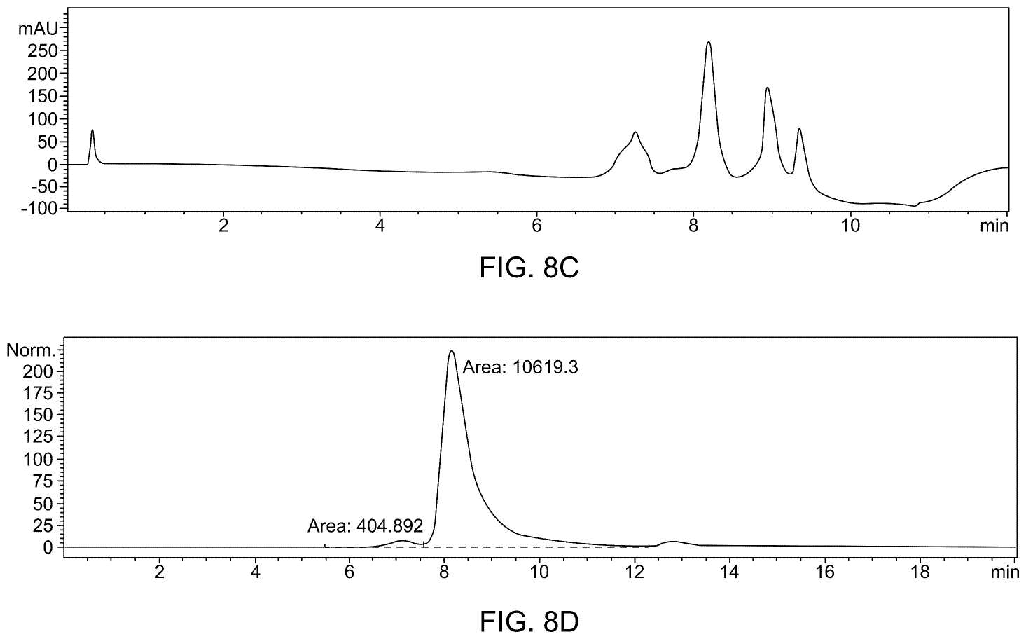

Methods: Rat cross-reactive nectin-4 ADCs carrying either RED-106 or vedotin linker-payloads were prepared (DAR depicted in FIGS. 8A-8D) and tested in a rat toxicity study.

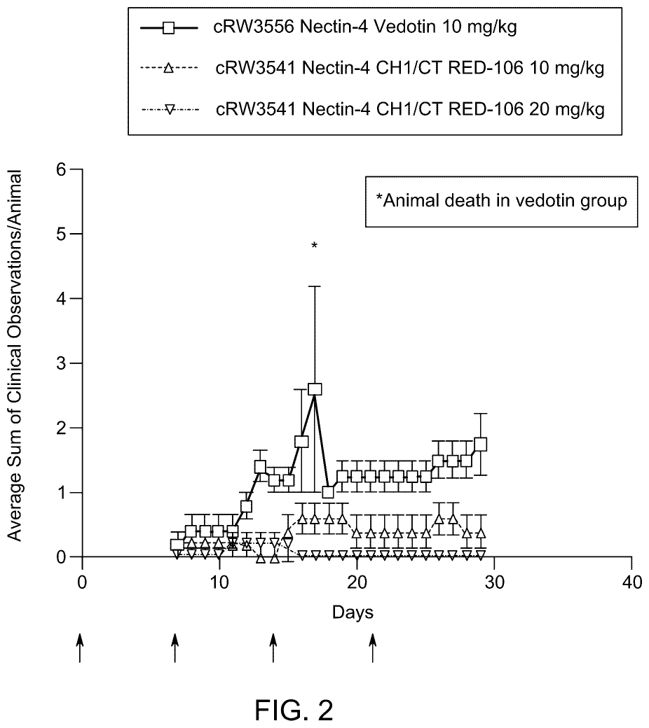

Results: Previous reports of the effect of the nectin-4 vedotin conjugate dosed at 10 mg/kg weekly for four doses had noted fur and skin toxicity and mortality. The fur and skin toxicity was attributed to the expression of the nectin-4 target antigen in skin and was considered to be an on-target toxicity. The previously observed effects were reproduced in this study, where 1 animal in the vedotin dosing group was euthanized in distress and all animals exhibited clinical signs of toxicity in fur and skin. By contrast, the effects of the RED-106 conjugate were absent or much less pronounced, with no mortalities-even at twice the dosing level as compared to the vedotin conjugate—and fewer signs of fur/skin toxicity (FIG. 2).

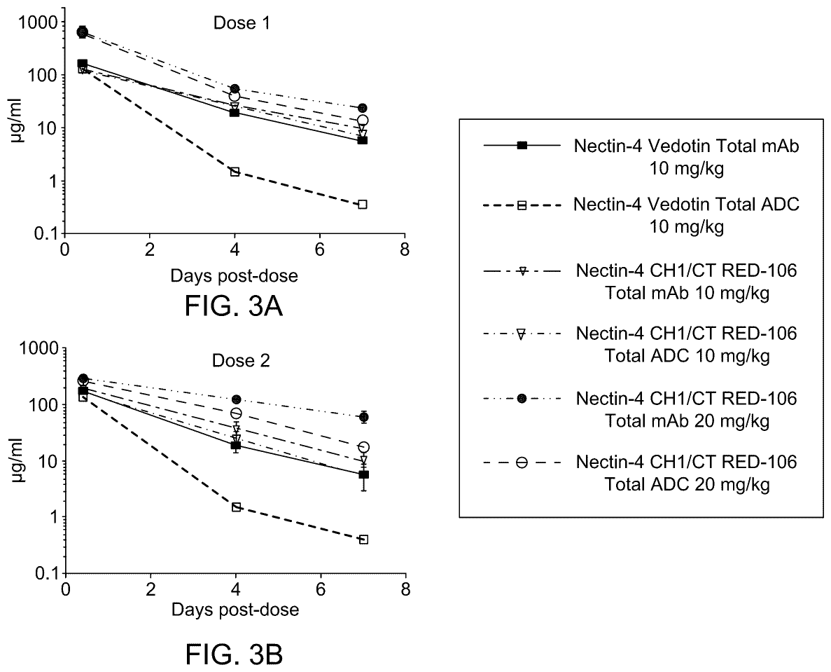

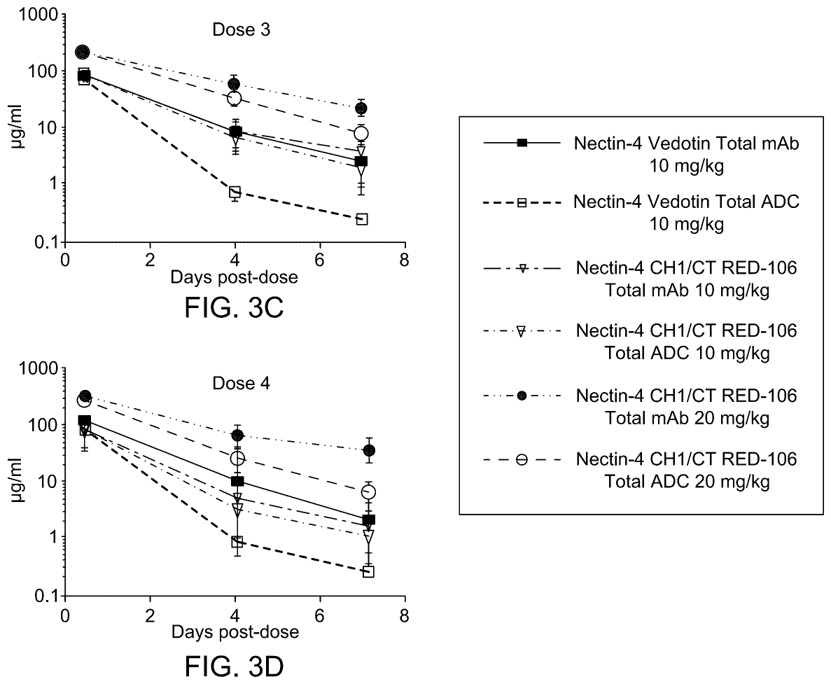

Further, toxicokinetic analysis of plasma samples from the animals confirmed dosing levels and exposure, and demonstrated improved stability of the RED-106 conjugate as compared to the vedotin ADC (FIGS. 3A-3D).

### Example 4: Tumor-Associated Calcium Signal Transducer 2 (TACSTD2) RED-106 ADC

### A. In Vitro Potency

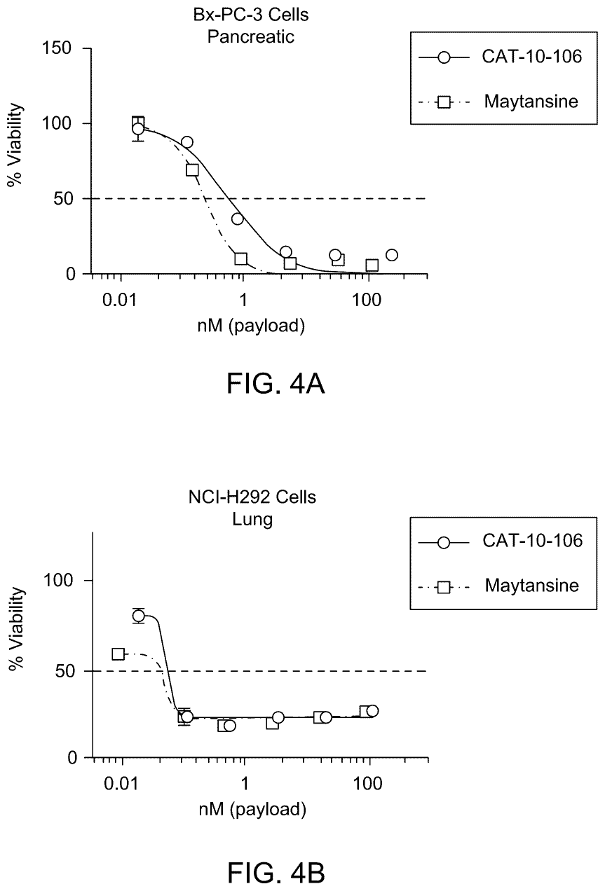

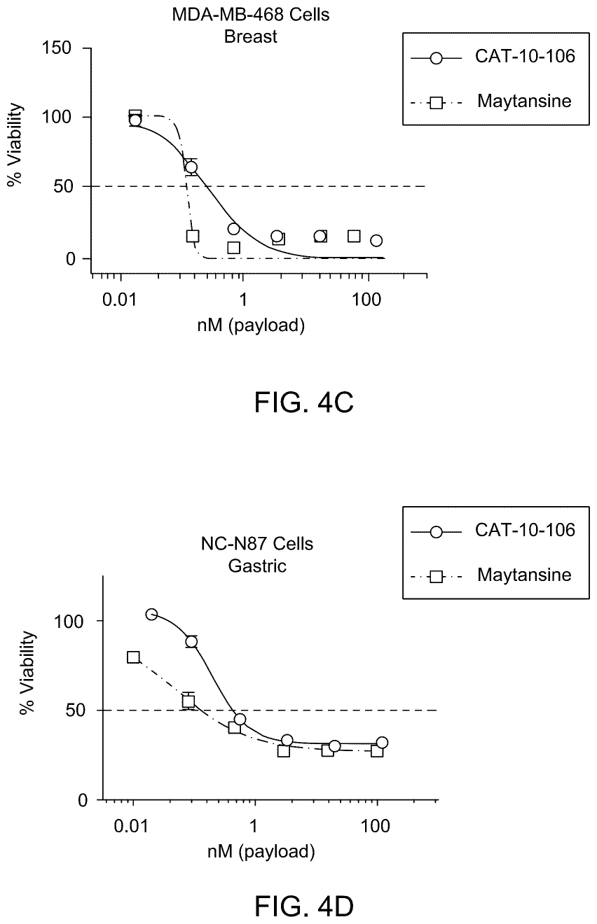

TACSTD2-expressing cell lines representing various solid tumor indications were treated with free maytansine and a TACSTD2 RED-106 ADC (also known as CAT-10⁻¹⁰⁶) (FIGS. 4A-4D) to test in vitro potency. The TACSTD2 ADC was equipotent to the free payload across the tested cell lines.

TABLE 10
CH1/CT RED-106 Conjugate Shows Equal Potency Compared to Cleavable
MMAE Conjugate Against NCI-H1781 Cells In Vitro.

| | Absolute IC50 (μM) | | | | | |
|---|---|---|---|---|---|---|
| | | | | Nectin-4 | | |
| | | | Nectin-4 | CH1/CT | Nectin-4 | |
| | Free | Free | CH1/CT | Cleavable | Camptothecin | |
| Cell line | MMAE | Camptothecin | RED-106 | MMAE | ADC | Cisplatin |
| NCI-H1781 | 0.0003 | 0.001 | 0.001 | 0.001 | 0.09 | 0.23 |

### B. In Vivo Efficacy

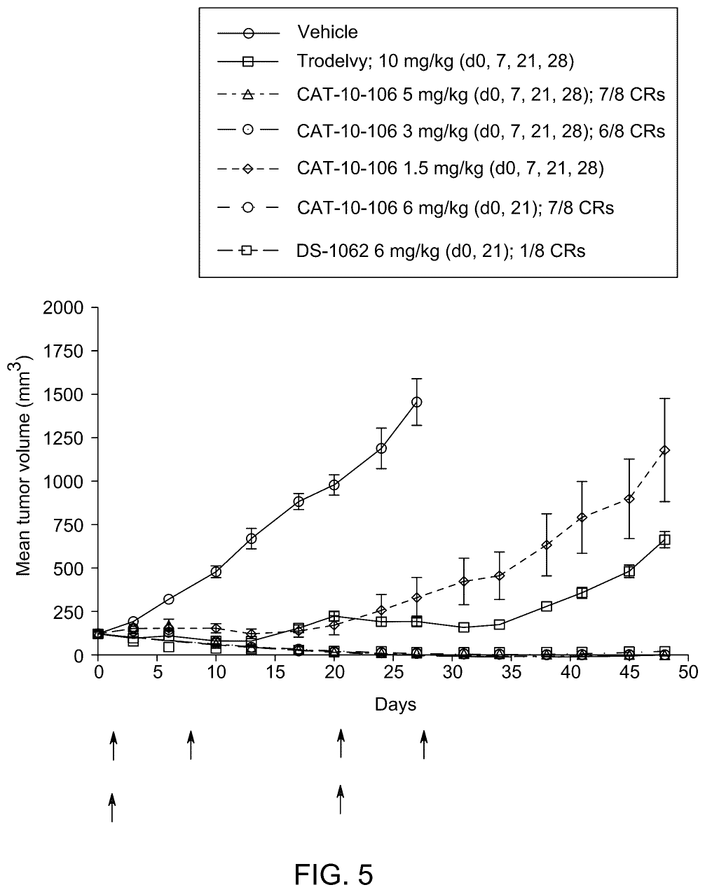

The TACSTD2-expressing lung xenograft model, NCI-H292, was used to test in vivo efficacy of TACSTD2-targeted ADCs, including a RED-106 conjugate (CAT-10⁻¹⁰⁶), Trodelvy, and DS-1062 (FIG. 5). Across a range of doses and dosing schedules, CAT-10⁻¹⁰⁶ showed equal or greater potency as compared to Trodelvy and DS-1062.

### C. Non-GLP Non-Human Primate Toxicity Study

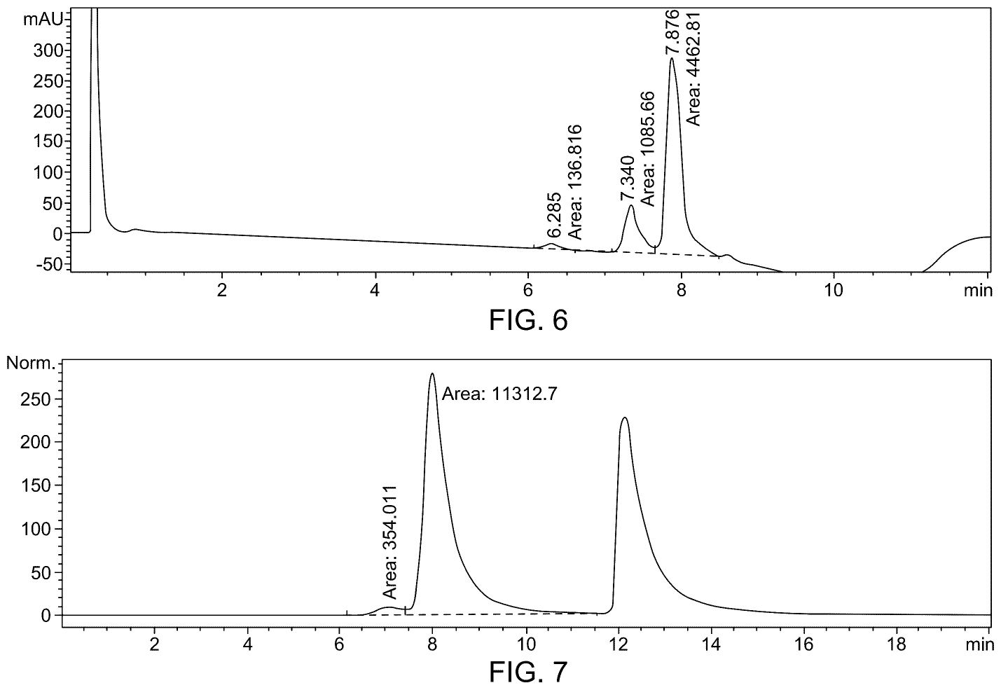

Cynomolgus cross-reactive TACSTD2 RED-106 ADC (CAT-10⁻¹⁰⁶) was prepared (FIG. 6 and FIG. 7) and tested in a non-human primate toxicity study. Previous reports of the preclinical and clinical effects of two other TACSTD2-targeted ADCs (DS-1062 and PF-06664178) had noted skin and mucosal toxicity. The skin and mucosal toxicity was attributed to the expression of the TACSTD2 target antigen in those tissues and was considered to be an on-target toxicity. By contrast, in this study, even when animals were dosed repeatedly at 5 mg/kg (for a total of 10 mg/kg ADC dose over 21 days), there were no treatment-related findings. Of particular importance is the lack of dermal observations (Table 11).

TABLE 11
Table 8468511
Individual Dermal Observations

| Test Article | (dosage) | 1F | 2F | 3F | 4F |
|---|---|---|---|---|---|
| cRW3543 | mg/kg/dose | 0 | 1.5 | 3 | 5 |

| Group/ | Animal | | | |
|---|---|---|---|---|
| Sex | Number | Observation | Phase | Day(s) |
| 1/F | P0001 | Test site A | | |
| | | edema, very slight | DSNG | 9 |
| | | erythema, very slight | DSNG | 2, 3, 11, 22, 23, 29 |
| | | erythema, well-defined | DSNG | 9, 10 |
| | | Test site B | | |
| | | edema, very slight | DSNG | 29 |
| | | edema, slight | DSNG | 30, 31 |
| | | erythema, very slight | DSNG | 29, 32 |
| | | erythema, well-defined | DSNG | 9-11 |
| | | erythema, moderate to severe | DSNG | 30, 31 |
| 1/F | P0002 | Test site A | | |
| | | erythema, very slight | DSNG | 22-25 |
| | | Test site B | | |
| | | edema, very slight | DSNG | 29-31 |
| | | erythema, very slight | DSNG | 29, 32 |
| | | erythema, well-defined | DSNG | 30, 31 |
| 1/F | P0003 | Test site B | | |
| | | edema, very slight | DSNG | 9, 10 |
| | | erythema, very slight | DSNG | 31, 32 |
| | | erythema, well-defined | DSNG | 9-11, 30 |
| 1/F | P0004 | Test site A | | |
| | | erythema, very slight | DSNG | 3, 22, 23 |
| | | Test site B | DSNG | 30, 31 |
| | | erythema, well-defined | | |
| 1/F | P0005 | Test site A | | |
| | | erythema, very slight | DSNG | 2, 3 |
| | | Test site B | | |
| | | edema, very slight | DSNG | 9-11 |
| 2/F | P0101 | Test site B | | |
| | | edema, very slight | DSNG | 9-11 |
| | | erythema, very slight | DSNG | 9-11, 29, 32 |
| | | erythema, well-defined | DSNG | 30, 31 |
| 2/F | P0102 | Test site B | | |
| | | edema, very slight | DSNG | 29-31 |
| | | erythema, very slight | DSNG | 29 |
| | | erythema, well-defined | DSNG | 30-32 |
| 2/F | P0103 | Test site B | | |
| | | erythema, very slight | DSNG | 9-11, 30-32 |
| 3/F | P0201 | Test site A | | |
| | | erythema, very slight | DSNG | 3 |
| | | Test site B | | |
| | | edema, very slight | DSNG | 9 |
| | | erythema, very slight | DSNG | 11, 32 |
| 3/F | P0202 | Test site A | | |
| | | erythema, very slight | DSNG | 4 |
| | | erythema, well-defined | DSNG | 2, 3 |
| | | Test site B | | |
| | | edema, very slight | DSNG | 29 |
| | | erythema, very slight | DSNG | 29-31 |
| 3/F | P0203 | Test site A | | |
| | | erythema, very slight | DSNG | 2 |
| | | erythema, well-defined | DSNG | 3, 4 |
| | | Test site B | | |
| | | edema, very slight | DSNG | 11, 29, 30 |
| | | edema, slight | DSNG | 9, 10 |
| | | erythema, very slight | DSNG | 3, 9, 10, 29-32 |
| | | erythema, well-defined | DSNG | 11 |
| 4/F | P0301 | Test site A | | |
| | | desquamation, yes | DSNG | 9-11, 22 |
| | | edema, very slight | DSNG | 29 |
| | | erythema, very slight | DSNG | 2-4, 22, 29, 30 |
| | | Test site B | | |
| | | edema, very slight | DSNG | 29, 30 |
| | | erythema, very slight | DSNG | 9-11, 29 |
| | | erythema, well-defined | DSNG | 30-32 |
| 4/F | P0302 | Test site A | | |
| | | atonia, yes | DSNG | 9-11, 22-24 |
| | | edema, very slight | DSNG | 2, 3 |
| | | erythema, very slight | DSNG | 2-4 |
| | | Test site B | | |
| | | atonia, yes | DSNG | 9-11, 22-24 |
| | | edema, very slight | DSNG | 29-31 |
| | | erythema, very slight | DSNG | 9-11, 29-32 |
| 4/F | P0303 | Test site A | | |
| | | erythema, very slight | DSNG | 2, 4 |
| | | erythema, well-defined | DSNG | 3 |
| | | Test site B | | |
| | | erythema, very slight | DSNG | 9 |
| 4/F | P0304 | Test site A | | |
| | | erythema, very slight | DSNG | 2-4 |
| 4/F | P0305 | Test site A | | |
| | | edema, very slight | DSNG | 2, 3 |
| | | erythema, very slight | DSNG | 2-4 |
| | | Test site B | | |
| | | ervthema. verv slight | DSNG | 10. 11 |

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

We claim:

1. A method of reducing toxicity associated with target-mediated cross-reactivity in a subject by administering an antibody-drug conjugate (ADC) of formula (I) to the subject,
wherein, the ADC of formula (I) is: 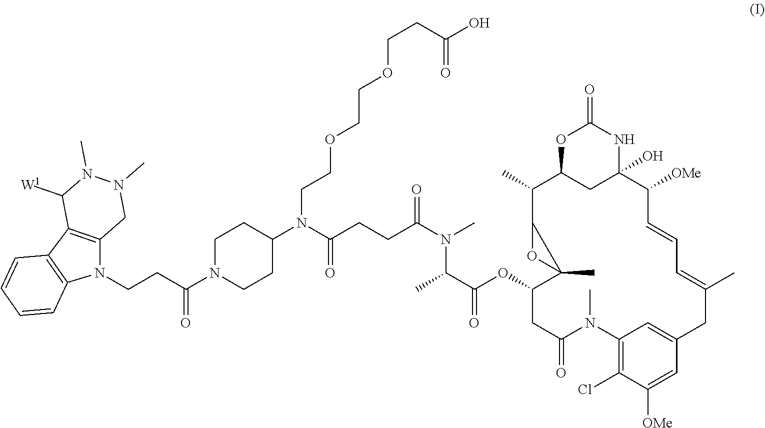
wherein W¹ is an antibody binding to an antigen; and
wherein the administering reduces the toxicity in the subject associated with target-mediated cross-reactivity of the ADC.

2. The method of claim 1, wherein the antigen is expressed in skin or mucosal epithelium of the subject.

3. The method of claim 2, wherein the antigen is selected from the group consisting of nectin-4, TACSTD2, EGFR, ERBB3, glycoprotein non-metastatic melanoma protein B (GPNMB), SLC39A6 (LIV-1), SLITRK6, GUCY2C, MUC1, NaPi2b, and cadherin 3.

4. The method of claim 1, wherein the antibody is an anti-nectin-4 antibody.

5. The method of claim 1, wherein the antibody is an anti-Tumor Associated Calcium Signal Transducer 2 (TACSTD2) antibody.

6. The method of claim 1, wherein the antibody comprising the sequence:
X¹(fGly′)X²Z²⁰X³Z³⁰
wherein
Z²⁰ is either a proline or alanine residue;
Z³⁰ is a basic amino acid or an aliphatic amino acid;
X¹ may be present or absent and, when present, can be any amino acid,
with the proviso that when the sequence is at the N-terminus of the antibody, X¹ is present; and
X² and X³ are each independently any amino acid.

7. The method of claim 1, wherein the antibody is an anti-Muc antibody.

8. The method of claim 1, wherein the antibody is an anti-NaPi2b antibody.

9. The method of claim 1, wherein the antibody binds to at least one target antigen expressed on a vital organ of the subject.

10. The method of claim 1, wherein the toxicity is reduced compared to when the subject is administered an antibody-drug conjugate targeting the same antigen and comprising a linker and a payload different from the ADC of formula (I).

11. The method of claim 1, wherein the subject has a cell proliferative disorder.

12. The method of any one of claims 1 to 11, wherein the wherein the antibody is an IgG1 antibody.

13. The method of claim 12, wherein the antibody is an IgG1 kappa antibody.

14. The method of any one of claims 1 to 13, wherein the antibody comprises an fGly′ residue, wherein fGly′ is an amino acid of the antibody coupled at W¹.

15. The method of claim 14, wherein the fGly′ is positioned at or near a C-terminus of a heavy chain constant region of the antibody.

16. The method of claim 14, wherein the fGly′ residue is positioned in a light chain constant region of the antibody.

17. The method of claim 14, wherein the fGly′ residue is positioned in a heavy chain CH1 region of the antibody.

18. The method of claim 14, wherein the fGly′ residue is positioned in a heavy chain CH2 region of the antibody.

19. The method of claim 14, wherein the fGly′ residue is positioned in a heavy chain CH3 region of the antibody.

20. The method of any one of claims 1-19, wherein the antibody-drug conjugate (ADC) of formula (I) is administered to the subject parenterally.

21. The method of any one of claims 1-19, wherein the antibody-drug conjugate (ADC) of formula (I) is administered to the subject non-parenterally.

22. The method of any one of claims 1-21, wherein the antibody is a monoclonal antibody.

23. The method of any one of claims 1-21, wherein the antibody is a humanized antibody.

24. The method of any one of claims 1-21, wherein the drug in the antibody-drug conjugate (ADC) of formula (I) is an anti-cancer drug.

25. The method of claim 24, wherein the anti-cancer drug comprises a maytansinoid.

26. The method of claim 1, wherein the toxicity is reduced in the subject by at least 2 folds when the ADC of Formula (I) is administered, as compared to administering the subject an antibody-drug conjugate targeting the same antigen and comprising a linker and a payload different from the ADC of formula (I).


SEQUENCE LISTING (220 sequences)

SEQ ID NO: 1 - protein, 5 aa, synthetic construct
NYGMN

SEQ ID NO: 2 - protein, 17 aa, synthetic construct
WINTYTGEPTYTDDFKG

SEQ ID NO: 3 - protein, 12 aa, synthetic construct
GGFGSSYWYFDV

SEQ ID NO: 4 - protein, 11 aa, synthetic construct
KASQDVSIAVA

SEQ ID NO: 5 - protein, 7 aa, synthetic construct
SASYRYT

SEQ ID NO: 6 - protein, 9 aa, synthetic construct
QQHYITPLT

SEQ ID NO: 7 - protein, 108 aa, synthetic construct
EIVLTQSPATLSLSPGERATLSCRASSSVSSSYLYWYQQKPGQAPRLWIYGTSNLASGVPARFSGSGSGTDYTLTISSLEPEDAAVYYCHQYAWSPPTFGQGTKLEIK

SEQ ID NO: 8 - protein, 107 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIK

SEQ ID NO: 9 - protein, 117 aa, synthetic construct
EVQLVQSGAEVKKPGATVKISCKVSGYTFTDHTMHWIKQRPGKGLEWMGYFYPRDDSTNYNEKFKGRVTLTADKSTDTAYMELSSLRSEDTAVYYCARGLRYALDYWGQGTLVTVSS

SEQ ID NO: 10 - protein, 5 aa, synthetic construct
DHTMH

SEQ ID NO: 11 - protein, 17 aa, synthetic construct
YFYPRDDSTNYNEKFKG

SEQ ID NO: 12 - protein, 7 aa, synthetic construct
GTSNLAS

SEQ ID NO: 13 - protein, 9 aa, synthetic construct
HQYAWSPPT

SEQ ID NO: 14 - protein, 12 aa, synthetic construct
RASSSVGSSNLY

SEQ ID NO: 15 - protein, 7 aa, synthetic construct
RSTKLAS

SEQ ID NO: 16 - protein, 9 aa, synthetic construct
HQYRWSPPT

SEQ ID NO: 17 - protein, 9 aa, synthetic construct
HQYSWSPPT

SEQ ID NO: 18 - DNA, 363 bp, synthetic construct
  source 1..363: mol_type = other DNA
caggtccaactgcagcaatctgggtctgagttgaagaagcctggggcctcagtgaaggtttcctgcaaggcttctggatacaccttcacaaactatggaatgaactgggtgaagcaggcccctggacaagggcttaaatggatgggctggataaacacctacactggagagccaacatatactgatgacttcaagggacggtttgccttctccttggacacctctgtcagcacggcatatctccagatcagcagcctaaaggctgacgacactgccgtgtatttctgtgcaagaggggggttcggtagtagctactggtacttcgatgtctggggccaagggtccctggtcaccgtctcctca

SEQ ID NO: 19 - DNA, 1017 bp, synthetic construct
  source 1..1017: mol_type = other DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttcactgtgtaccccttctagaggatcctga

SEQ ID NO: 20 - DNA, 321 bp, synthetic construct
  source 1..321: mol_type = other DNA
gacatccagctgacccagtctccatcctccctgtctgcatctgtaggagacagagtcagcatcacctgcaaggccagtcaggatgtgagtattgctgtagcctggtatcagcagaaaccagggaaagcccctaagctcctgatctactcggcatcctaccggtacactggagtccctgataggttcagtggcagtggatctgggacagatttcactctcaccatcagcagtctgcaacctgaagattttgcagtttattactgtcagcaacattatattactccgctcacgttcggtgctgggaccaaggtggagatcaaa

SEQ ID NO: 21 - DNA, 324 bp, Homo sapiens
  source 1..324: mol_type = genomic DNA
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 22 - protein, 214 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 6 aa, synthetic construct
ICTPAR

SEQ ID NO: 24 - protein, 6 aa, synthetic construct
LCTPSK

SEQ ID NO: 25 - protein, 6 aa, synthetic construct
MCTPSK

SEQ ID NO: 26 - protein, 6 aa, synthetic construct
VCTPSK

SEQ ID NO: 27 - protein, 6 aa, synthetic construct
LCSPSK

SEQ ID NO: 28 - protein, 6 aa, synthetic construct
LCAPSK

SEQ ID NO: 29 - protein, 6 aa, synthetic construct
LCVPSK

SEQ ID NO: 30 - protein, 6 aa, synthetic construct
LCGPSK

SEQ ID NO: 31 - protein, 6 aa, synthetic construct
LCTPSA

SEQ ID NO: 32 - protein, 6 aa, synthetic construct
ICTPAA

SEQ ID NO: 33 - protein, 6 aa, synthetic construct
MCTPSA

SEQ ID NO: 34 - protein, 6 aa, synthetic construct
VCTPSA

SEQ ID NO: 35 - protein, 6 aa, synthetic construct
LCSPSA

SEQ ID NO: 36 - protein, 6 aa, synthetic construct
LCAPSA

SEQ ID NO: 37 - protein, 6 aa, synthetic construct
LCVPSA

SEQ ID NO: 38 - protein, 6 aa, synthetic construct
LCGPSA

SEQ ID NO: 39 - protein, 16 aa, synthetic construct
  MOD_RES 10: note = FORMYLATED
SLSLSPGSLGTPSRGS

SEQ ID NO: 40 - protein, 8 aa, synthetic construct
SLSLSPGK

SEQ ID NO: 41 - protein, 6 aa, synthetic construct
KVDNAL

SEQ ID NO: 42 - protein, 6 aa, synthetic construct
QSGNSQ

SEQ ID NO: 43 - protein, 17 aa, synthetic construct
  MOD_RES 7: note = FORMYLATED
KVDNALGTPSRQSGNSQ

SEQ ID NO: 44 - protein, 6 aa, synthetic construct
SWNSGA

SEQ ID NO: 45 - protein, 6 aa, synthetic construct
GVHTFP

SEQ ID NO: 46 - protein, 18 aa, synthetic construct
  MOD_RES 8: note = FORMYLATED
SWNSGALGTPSRGVHTFP

SEQ ID NO: 47 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 6 aa, Homo sapiens
ASTKGP

SEQ ID NO: 49 - protein, 7 aa, Homo sapiens
KSTSGGT

SEQ ID NO: 50 - protein, 4 aa, Homo sapiens
PEPV

SEQ ID NO: 51 - protein, 8 aa, Homo sapiens
NSGALTSG

SEQ ID NO: 52 - protein, 21 aa, Homo sapiens
NSGALTSGVHTFPAVLQSSGL

SEQ ID NO: 53 - protein, 5 aa, Homo sapiens
QSSGL

SEQ ID NO: 54 - protein, 4 aa, Homo sapiens
TQTY

SEQ ID NO: 55 - protein, 5 aa, Homo sapiens
HKPSN

SEQ ID NO: 56 - protein, 22 aa, Homo sapiens
EPKSCDKTHTCPPCPAPELLGG

SEQ ID NO: 57 - protein, 5 aa, Homo sapiens
FPPKP

SEQ ID NO: 58 - protein, 5 aa, Homo sapiens
ISRTP

SEQ ID NO: 59 - protein, 9 aa, Homo sapiens
DVSHEDPEV

SEQ ID NO: 60 - protein, 7 aa, Homo sapiens
SHEDPEV

SEQ ID NO: 61 - protein, 9 aa, Homo sapiens
DGVEVHNAK

SEQ ID NO: 62 - protein, 5 aa, Homo sapiens
QYNST

SEQ ID NO: 63 - protein, 5 aa, Homo sapiens
VLTVL

SEQ ID NO: 64 - protein, 7 aa, Homo sapiens
NKALPAP

SEQ ID NO: 65 - protein, 9 aa, Homo sapiens
SKAKGQPRE

SEQ ID NO: 66 - protein, 7 aa, Homo sapiens
KAKGQPR

SEQ ID NO: 67 - protein, 10 aa, Homo sapiens
PPSRKELTKN

SEQ ID NO: 68 - protein, 5 aa, Homo sapiens
YPSDI

SEQ ID NO: 69 - protein, 7 aa, Homo sapiens
NGQPENN

SEQ ID NO: 70 - protein, 10 aa, Homo sapiens
TPPVLDSDGS

SEQ ID NO: 71 - protein, 19 aa, Homo sapiens
HEALHNHYTQKSLSLSPGK

SEQ ID NO: 72 - protein, 6 aa, Homo sapiens
SLSPGK

SEQ ID NO: 73 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 74 - skipped

SEQ ID NO: 75 - protein, 12 aa, Homo sapiens
PCSRSTSESTAA

SEQ ID NO: 76 - protein, 5 aa, Homo sapiens
FPEPV

SEQ ID NO: 77 - protein, 12 aa, Homo sapiens
SGALTSGVHTFP

SEQ ID NO: 78 - protein, 6 aa, Homo sapiens
QSSGLY

SEQ ID NO: 79 - protein, 5 aa, Homo sapiens
VAGPS

SEQ ID NO: 80-82 - skipped (3 entries)

SEQ ID NO: 83 - protein, 5 aa, Homo sapiens
VLTVV

SEQ ID NO: 84 - protein, 7 aa, Homo sapiens
NKGLPAP

SEQ ID NO: 85 - protein, 9 aa, Homo sapiens
SKTKGQPRE

SEQ ID NO: 86 - protein, 5 aa, Homo sapiens
MTKNQ

SEQ ID NO: 87-88 - skipped (2 entries)

SEQ ID NO: 89 - protein, 10 aa, Homo sapiens
TPPMLDSDGS

SEQ ID NO: 90 - protein, 4 aa, Homo sapiens
GNVF

SEQ ID NO: 91 - skipped

SEQ ID NO: 92 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 93 - skipped

SEQ ID NO: 94 - protein, 10 aa, Homo sapiens
PCSRSTSGGT

SEQ ID NO: 95 - skipped

SEQ ID NO: 96 - protein, 19 aa, Homo sapiens
SGALTSGVHTFPAVLQSSG

SEQ ID NO: 97 - skipped

SEQ ID NO: 98 - protein, 10 aa, Homo sapiens
RVELKTPLGD

SEQ ID NO: 99 - protein, 7 aa, Homo sapiens
CPRCPKP

SEQ ID NO: 100 - protein, 21 aa, Homo sapiens
PKSCDTPPPCPRCPAPELLGG

SEQ ID NO: 101-105 - skipped (5 entries)

SEQ ID NO: 106 - protein, 10 aa, Homo sapiens
PPSREEMTKN

SEQ ID NO: 107 - skipped

SEQ ID NO: 108 - protein, 8 aa, Homo sapiens
SSGQPENN

SEQ ID NO: 109 - skipped

SEQ ID NO: 110 - protein, 7 aa, Homo sapiens
HEALHNR

SEQ ID NO: 111 - skipped

SEQ ID NO: 112 - protein, 326 aa, Homo sapiens
STKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 113 - protein, 5 aa, Homo sapiens
STKGP

SEQ ID NO: 114-117 - skipped (4 entries)

SEQ ID NO: 118 - protein, 11 aa, Homo sapiens
CPAPEFLGGPS

SEQ ID NO: 119 - skipped

SEQ ID NO: 120 - protein, 9 aa, Homo sapiens
DVSQEDPEV

SEQ ID NO: 121 - skipped

SEQ ID NO: 122 - protein, 7 aa, Homo sapiens
NKGLPSS

SEQ ID NO: 123 - protein, 10 aa, Homo sapiens
SKAKGQPREP

SEQ ID NO: 124 - protein, 10 aa, Homo sapiens
PPSQEEMTKN

SEQ ID NO: 125-127 - skipped (3 entries)

SEQ ID NO: 128 - protein, 19 aa, Homo sapiens
HEALHNHYTQKSLSLSLGK

SEQ ID NO: 129 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPDRDLCGCYSVSSVLSGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 130 - protein, 13 aa, Homo sapiens
ASPTSPKVFPLSL

SEQ ID NO: 131 - protein, 5 aa, Homo sapiens
QPDGN

SEQ ID NO: 132 - protein, 10 aa, Homo sapiens
VQGFFPQEPL

SEQ ID NO: 133 - protein, 11 aa, Homo sapiens
SGQGVTARNFP

SEQ ID NO: 134 - protein, 7 aa, Homo sapiens
SGDLYTT

SEQ ID NO: 135 - protein, 4 aa, Homo sapiens
PATQ

SEQ ID NO: 136 - protein, 4 aa, Homo sapiens
HRPA

SEQ ID NO: 137 - protein, 5 aa, Homo sapiens
LLGSE

SEQ ID NO: 138 - protein, 8 aa, Homo sapiens
GLRDASGV

SEQ ID NO: 139 - protein, 10 aa, Homo sapiens
SSGKSAVQGP

SEQ ID NO: 140 - protein, 4 aa, Homo sapiens
GCYS

SEQ ID NO: 141 - protein, 4 aa, Homo sapiens
CAEP

SEQ ID NO: 142 - protein, 24 aa, Homo sapiens
SGNTFRPEVHLLPPPSEELALNEL

SEQ ID NO: 143 - protein, 5 aa, Homo sapiens
ARGFS

SEQ ID NO: 144 - protein, 11 aa, Homo sapiens
QGSQELPREKY

SEQ ID NO: 145 - protein, 4 aa, Homo sapiens
AAED

SEQ ID NO: 146 - protein, 4 aa, Homo sapiens
HEAL

SEQ ID NO: 147 - protein, 25 aa, Homo sapiens
IDRLAGKPTHVNVSVVMAEVDGTCY

SEQ ID NO: 148 - skipped

SEQ ID NO: 149 - protein, 11 aa, synthetic construct
NSGALCTPSRG

SEQ ID NO: 150 - skipped

SEQ ID NO: 151 - protein, 5 aa, synthetic construct
CTPSR

SEQ ID NO: 152-153 - skipped (2 entries)

SEQ ID NO: 154 - protein, 9 aa, synthetic construct
NLCTPSRAP

SEQ ID NO: 155-158 - skipped (4 entries)

SEQ ID NO: 159 - protein, 10 aa, synthetic construct
KAKGLCTPSR

SEQ ID NO: 160 - skipped

SEQ ID NO: 161 - protein, 5 aa, synthetic construct
LCTPS

SEQ ID NO: 162 - skipped

SEQ ID NO: 163 - protein, 107 aa, Homo sapiens [sequence omitted]

SEQ ID NO: 164 - protein, 6 aa, Homo sapiens
RTVAAP

SEQ ID NO: 165 - protein, 5 aa, Homo sapiens
YPREA

SEQ ID NO: 166 - protein, 4 aa, Homo sapiens
PREA

SEQ ID NO: 167 - protein, 8 aa, Homo sapiens
DNALQSGN

SEQ ID NO: 168 - protein, 9 aa, Homo sapiens
TEQDSKDST

SEQ ID NO: 169 - protein, 6 aa, Homo sapiens
HQGLSS

SEQ ID NO: 170 - protein, 4 aa, Homo sapiens
RGEC

SEQ ID NO: 171 - protein, 6 aa, Homo sapiens
QPKAAP

SEQ ID NO: 172 - protein, 7 aa, Homo sapiens
DFYPGAV

SEQ ID NO: 173 - protein, 8 aa, Homo sapiens
DSSPVKAG

SEQ ID NO: 174 - protein, 6 aa, Homo sapiens
APTECS

SEQ ID NO: 175 - protein, 105 aa, Rattus norvegicus
QPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 176 - protein, 99 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVE

SEQ ID NO: 177 - protein, 8 aa, synthetic construct
GALTSGVH

SEQ ID NO: 178 - protein, 11 aa, synthetic construct
GALCTPSRGVH

SEQ ID NO: 179 - skipped

SEQ ID NO: 180 - protein, 9 aa, synthetic construct
SLCTPSRGS

SEQ ID NO: 181-183 - skipped (3 entries)

SEQ ID NO: 184 - protein, 18 aa, synthetic construct
KVDNALLCTPSRQSGNSQ

SEQ ID NO: 185 - protein, 108 aa, synthetic construct
EIVLTQSPATLSLSPGERATLSCRASSSVGSSNLYWYQQKPGQAPRLWIYRSTKLASGVPARFSGSGSGTDYTLTISSLEPEDAAVYYCHQYRWSPPTFGQGTKLEIK

SEQ ID NO: 186 - protein, 108 aa, synthetic construct
EIVLTQSPATLSLSPGERATLSCRASSSVSSSYLYWYQQKPGQAPRLWIIGTSNLASGVPARFSGSGSGTDYTLTISSLEPEDAAVYYCHQYSWSPPTFGQGTKLEIK

SEQ ID NO: 187 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 188 - protein, 107 aa, Homo sapiens
RTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 189 - protein, 107 aa, Homo sapiens
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 190 - protein, 107 aa, Mus musculus
RADAAPTVSIFPPSMEQLTSGGATVVCFVNNFYPRDISVKWKIDGSEQRDGVLDSVTDQDSKDSTYSMSSTLSLTKVEYERHNLYTCEVVHKTSSSPVVKSFNRNEC

SEQ ID NO: 191 - protein, 6 aa, synthetic construct
  VARIANT 2: note = Wherein X can be C or S
LXTPSR

SEQ ID NO: 192 - protein, 6 aa, synthetic construct
  MOD_RES 2: note = FORMYLATED
LGTPSR

SEQ ID NO: 193 - protein, 323 aa, Homo sapiens
MARGPGLAPPPLRLPLLLLVLAAVTGHTAAQDNCTCPTNKMTVCSPDGPGGRCQCRALGSGMAVDCSTLTSKCLLLKARMSAPKNARTLVRPSEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGDVDIGDAAYYFERDIKGESLFQGRGGLDLRVRGEPLQVERTLIYYLDEIPPKFSMKRLTAGLIAVIVVVVVALVAGMAVLVITNRRKSGKYKKVEIKELGELRKEPSL

SEQ ID NO: 194 - skipped

SEQ ID NO: 195 - protein, 459 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSLCTPSRGS

SEQ ID NO: 196 - protein, 121 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 197 - protein, 6 aa, synthetic construct
LCTPSR

SEQ ID NO: 198 - protein, 10 aa, synthetic construct
QKSLSLSPGK

SEQ ID NO: 199 - protein, 7 aa, synthetic construct
GYTFTDH

SEQ ID NO: 200 - protein, 8 aa, synthetic construct
GYTFTDHT

SEQ ID NO: 201 - protein, 6 aa, synthetic construct
YPRDDS

SEQ ID NO: 202 - protein, 8 aa, synthetic construct
FYPRDDST

SEQ ID NO: 203 - protein, 8 aa, synthetic construct
GLRYALDY

SEQ ID NO: 204 - protein, 10 aa, synthetic construct
ARGLRYALDY

SEQ ID NO: 205 - protein, 12 aa, synthetic construct
RASSSVSSSYLY

SEQ ID NO: 206 - protein, 7 aa, synthetic construct
SSVSSSY

SEQ ID NO: 207 - protein, 7 aa, synthetic construct
SSVGSSN

SEQ ID NO: 208 - protein, 13 aa, synthetic construct
RASSSVGSSSYLY

SEQ ID NO: 209 - protein, 11 aa, synthetic construct
GRTSSTNKLAS

SEQ ID NO: 210 - protein, 11 aa, synthetic construct
HQYARSWSPPT

SEQ ID NO: 211 - DNA, 351 bp, synthetic construct
  source 1..351: mol_type = other DNA
gaggtccagctggtacagtctggggctgaggtgaagaagcctggggctacagtgaaaatctcctgcaaggtttctggatacaccttcaccgaccataccatgcactggatcaaacagcgacctggaaaagggcttgagtggatgggatacttctaccctagagatgattccacaaattacaacgagaagttcaagggcagagtcacccttaccgcggacaaatctacagacacagcctacatggagctgagcagcctgagatctgaggacacggccgtgtattactgtgcgcgtggtcttcgatacgctcttgactactggggccaaggaaccctggtcaccgtctcctca

SEQ ID NO: 212 - DNA, 324 bp, synthetic construct
  source 1..324: mol_type = other DNA
gaaattgtgttgacacagtctccagccaccctgtctttgtctccaggggaaagagccaccctctcctgcagggccagttcaagtgttagcagcagctacttatactggtaccagcagaaacctggccaggctcccaggctctggatctatggtacctccaaccttgcctccggcgtcccagcaaggttcagtggcagtgggtctgggacagactacactctcaccatcagctccctggagcctgaagatgcggcagtttattactgtcaccaatacgcctggtccccgccgacgttcggccaagggaccaagttggaaatcaaa

SEQ ID NO: 213 - DNA, 324 bp, synthetic construct
  source 1..324: mol_type = other DNA
gaaattgtgttgacacagtctccagccaccctgtctttgtctccaggggaaagagccaccctctcctgcagggccagttcaagtgttggcagcagcaacttatactggtaccagcagaaacctggccaggctcccaggctctggatctataggtccaccaaacttgcctccggcgtcccagcaaggttcagtggcagtgggtctgggacagactacactctcaccatcagctccctggagcctgaagatgcggcagtttattactgtcaccaatacagatggtccccgccgacgttcggccaagggaccaagttggaaatcaaa

SEQ ID NO: 214 - DNA, 324 bp, synthetic construct
  source 1..324: mol_type = other DNA
gaaattgtgttgacacagtctccagccaccctgtctttgtctccaggggaaagagccaccctctcctgcagggccagttcaagtgttagcagcagctacttatactggtaccagcagaaacctggccaggctcccaggctctggatcattggtacctccaaccttgcctccggcgtcccagcaaggttcagtggcagtgggtctgggacagactacactctcaccatcagctccctggagcctgaagatgcggcagtttattactgtcaccaatactcctggtccccgccgacgttcggccaagggaccaagttggaaatcaaa

SEQ ID NO: 215 - protein, 1258 aa, synthetic construct
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSSVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANLREF

SEQ ID NO: 216 - protein, 6 aa, synthetic construct
VCTPSR

SEQ ID NO: 217 - protein, 6 aa, synthetic construct
LCSPSR

SEQ ID NO: 218 - protein, 6 aa, synthetic construct
LCAPSR

SEQ ID NO: 219 - protein, 6 aa, synthetic construct
LCVPSR

SEQ ID NO: 220 - protein, 6 aa, synthetic construct
LCGPSR